SEQUENCE LISTING (38 sequences)

SEQ ID NO: 1 - DNA, 19 bp, synthetic construct
  misc_feature 1..19: note = eis Forward Primer
  source 1..19: mol_type = other DNA
tgtcgggtacctttcgagc

SEQ ID NO: 2 - DNA, 19 bp, synthetic construct
  misc_feature 1..19: note = eis Reverse Primer
  source 1..19: mol_type = other DNA
tccatgtacagcgccatcc

SEQ ID NO: 3 - DNA, 19 bp, synthetic construct
  misc_feature 1..19: note = embB Forward Primer
  source 1..19: mol_type = other DNA
cgccgtggtgatattcggc

SEQ ID NO: 4 - DNA, 19 bp, synthetic construct
  misc_feature 1..19: note = embB Reverse Primer
  source 1..19: mol_type = other DNA
gcacaccgtagctggagac

SEQ ID NO: 5 - DNA, 20 bp, synthetic construct
  misc_feature 1..20: note = rrs Forward Primer
  source 1..20: mol_type = other DNA
ctctgggcagtaactgacgc

SEQ ID NO: 6 - DNA, 19 bp, synthetic construct
  misc_feature 1..19: note = rrs Reverse Primer
  source 1..19: mol_type = other DNA
gagtgttgcctcaggaccc

SEQ ID NO: 7 - DNA, 19 bp, synthetic construct
  misc_feature 1..19: note = rv0678 Forward Primer
  source 1..19: mol_type = other DNA
gctcgtccttcacttcgcc

SEQ ID NO: 8 - DNA, 19 bp, synthetic construct
  misc_feature 1..19: note = rv0678 Reverse Primer
  source 1..19: mol_type = other DNA
atcagtcgtcctctccggt

SEQ ID NO: 9 - DNA, 19 bp, synthetic construct
  misc_feature 1..19: note = fabG1 Forward Primer
  source 1..19: mol_type = other DNA
cttttgcacgcaattgcgc

SEQ ID NO: 10 - DNA, 19 bp, synthetic construct
  misc_feature 1..19: note = fabG1 Reverse Primer
  source 1..19: mol_type = other DNA
agcagtcctgtcatgtgcg

SEQ ID NO: 11 - DNA, 19 bp, synthetic construct
  misc_feature 1..19: note = gyrA Forward Primer
  source 1..19: mol_type = other DNA
tgacagacacgacgttgcc

SEQ ID NO: 12 - DNA, 19 bp, synthetic construct
  misc_feature 1..19: note = gyrA Reverse Primer
  source 1..19: mol_type = other DNA
cgatcgctagcatgttggc

SEQ ID NO: 13 - DNA, 20 bp, synthetic construct
  misc_feature 1..20: note = rpoB Forward Primer
  source 1..20: mol_type = other DNA
tcatcatcaacgggaccgag

SEQ ID NO: 14 - DNA, 20 bp, synthetic construct
  misc_feature 1..20: note = rpoB Reverse Primer
  source 1..20: mol_type = other DNA
acacgatctcgtcgctaacc

SEQ ID NO: 15 - DNA, 19 bp, synthetic construct
  misc_feature 1..19: note = ethA Forward Primer
  source 1..19: mol_type = other DNA
tggatccatgaccgagcac

SEQ ID NO: 16 - DNA, 19 bp, synthetic construct
  misc_feature 1..19: note = eth A Reverse Primer
  source 1..19: mol_type = other DNA
gtccaggaggcattggtgt

SEQ ID NO: 17 - DNA, 19 bp, synthetic construct
  misc_feature 1..19: note = rplC Forward Primer
  source 1..19: mol_type = other DNA
agtacaaggactcgcggga

SEQ ID NO: 18 - DNA, 18 bp, synthetic construct
  misc_feature 1..18: note = rplC Reverse Primer
  source 1..18: mol_type = other DNA
tcgagtgggtaccctggc

SEQ ID NO: 19 - DNA, 19 bp, synthetic construct
  misc_feature 1..19: note = KatG Forward Primer
  source 1..19: mol_type = other DNA
ctgtggccggtcaagaaga

SEQ ID NO: 20 - DNA, 21 bp, synthetic construct
  misc_feature 1..21: note = KatG Reverse Primer redesigned
  source 1..21: mol_type = other DNA
ggatctggctcttaaggctgg

SEQ ID NO: 21 - DNA, 19 bp, synthetic construct
  misc_feature 1..19: note = gidB Forward Primer
  source 1..19: mol_type = other DNA
tgacacagacctcacgagc

SEQ ID NO: 22 - DNA, 19 bp, synthetic construct
  misc_feature 1..19: note = gidB Reverse Primer
  source 1..19: mol_type = other DNA
gcccttctgattcgcgatg

SEQ ID NO: 23 - DNA, 22 bp, synthetic construct
  misc_feature 1..22: note = inhA Forward Primer redesigned inhA FW 6
  source 1..22: mol_type = other DNA
cggattctggttagcggaatca

SEQ ID NO: 24 - DNA, 19 bp, synthetic construct
  misc_feature 1..19: note = inhA Reverse Primer
  source 1..19: mol_type = other DNA
ggcgtagatgatgtcaccc

SEQ ID NO: 25 - DNA, 17 bp, synthetic construct
  misc_feature 1..17: note = rrl Forward Primer
  source 1..17: mol_type = other DNA
ggtccgtgcgaagtcgc

SEQ ID NO: 26 - DNA, 18 bp, synthetic construct
  misc_feature 1..18: note = rrl Reverse Primer
  source 1..18: mol_type = other DNA
tgaacccgtgttctgcgg

SEQ ID NO: 27 - DNA, 19 bp, synthetic construct
  misc_feature 1..19: note = pncA Forward Primer
  source 1..19: mol_type = other DNA
tcaccggacggatttgtcg

SEQ ID NO: 28 - DNA, 19 bp, synthetic construct
  misc_feature 1..19: note = pncA Reverse Primer
  source 1..19: mol_type = other DNA
tccagatcgcgatggaacg

SEQ ID NO: 29 - DNA, 18 bp, synthetic construct
  misc_feature 1..18: note = rpsL Forward Primer
  source 1..18: mol_type = other DNA
gcggcgggtattgtggtt

SEQ ID NO: 30 - DNA, 18 bp, synthetic construct
  misc_feature 1..18: note = rpsL Reverse Primer
  source 1..18: mol_type = other DNA
taaccggcgcttctcacc

SEQ ID NO: 31 - DNA, 18 bp, synthetic construct
  misc_feature 1..18: note = tlyA Forward Primer
  source 1..18: mol_type = other DNA
cgttgatgcgcagcgatc

SEQ ID NO: 32 - DNA, 18 bp, synthetic construct
  misc_feature 1..18: note = tlyA Reverse Primer
  source 1..18: mol_type = other DNA
ggtctcggtggcttcgtc

SEQ ID NO: 33 - DNA, 19 bp, synthetic construct
  misc_feature 1..19: note = KatG Reverse Primer initial
  source 1..19: mol_type = other DNA
tgcccggatctggctctta

SEQ ID NO: 34 - DNA, 19 bp, synthetic construct
  misc_feature 1..19: note = inhA Forward Primer initial
  source 1..19: mol_type = other DNA
gggcgctgcaatttatccc

SEQ ID NO: 35 - DNA, 20 bp, synthetic construct
  misc_feature 1..20: note = inhA redesigned Forward Primer inhA FW 2
  source 1..20: mol_type = other DNA
acggcaaacggattctggtt

SEQ ID NO: 36 - DNA, 23 bp, synthetic construct
  misc_feature 1..23: note = inhA redesigned Forward Primer inhA FW 8
  source 1..23: mol_type = other DNA
ttctggttagcggaatcatcacc

SEQ ID NO: 37 - DNA, 22 bp, synthetic construct
  misc_feature 1..22: note = inhA redesigned Forward Primer inhA FW 9
  source 1..22: mol_type = other DNA
ctggttagcggaatcatcaccg

SEQ ID NO: 38 - DNA, 21 bp, synthetic construct
  source 1..21: mol_type = other DNA
  misc_feature 1..21: note = inhA redesigned Forward Primer inhA FW 11
ttagcggaatcatcaccgact

(19) United States
(12) Patent Application Publication
(10) Pub. No.: US 2025/0188550 A1
(43) Pub. Date: Jun. 12, 2025

(54) METHODS AND COMPOSITIONS FOR DRUG RESISTANCE SCREENING

(71) Applicants: Quadram Institute Bioscience, Norwich (GB); Oxford Nanopore Technologies PLC, Oxford (GB)

(72) Inventors: Justin Joseph O'GRADY, Oxford (GB); Gemma Louise KAY, Oxford (GB); Alp AYDIN, Oxford (GB)

(21) Appl. No.: 18/844,944

(22) PCT Filed: Mar. 7, 2023
(86) PCT No.: PCT/GB2023/050525

§ 371 Date: Sep. 6, 2024

(30) Foreign Application Priority Data
Mar. 8, 2022 (GB) 2203218.9

(51) Int. Cl. C12Q 1/689; C12Q 1/686

(52) U.S. Cl. CPC C12Q 1/689; C12Q 1/686; C12Q 2600/16

(57) ABSTRACT

The disclosure relates to novel primers, and their use to detect the presence of drug resistance mutations in a sample from a subject with suspected or confirmed Tuberculosis.

### CROSS-REFERENCE TO RELATED APPLICATIONS

The present application is a filing under 35 U.S.C. 371 as the National Stage of International Application No. PCT/GB2023/050525, filed Mar. 7, 2023, entitled “METHODS AND COMPOSITIONS FOR DRUG RESISTANCE SCREENING,” which claims priority to United Kingdom Application No. 2203218.9 filed with the Intellectual Property Office of United Kingdom on Mar. 8, 2022, both of which are incorporated herein by reference in their entirety for all purposes.

### INCORPORATION BY REFERENCE OF MATERIAL IN .XML ST26 TEXT FILE

This application incorporates by reference the Sequence Listing contained in the following .xml ST26 text file being submitted concurrently herewith:
File name: 4906-00200 1866189PP US ST26 Sequence Listing; created on Aug. 14, 2024; and having a file size of 48 KB.

The information in the Sequence Listing is incorporated herein in its entirety for all purposes.

### FIELD OF THE INVENTION

The invention to which this application relates is a new diagnostic methodology and primers and/or drug susceptibility testing (DST) assay. In particular, the present invention relates to novel primers and their use in a method of identifying and/or detecting the presence of drug resistance mutations in a sample from subjects with suspected or confirmed Tuberculosis, with a particular focus on a novel group of primers for use in a single multiplex PCR reaction to detect the presence of one or more drug resistance mutations in a sample from subjects with suspected or confirmed Tuberculosis.

### BACKGROUND

### Mycobacteria and Tuberculosis

Tuberculosis (TB), caused primarily by Mycobacterium tuberculosis^{1,2}, is a disease of global health importance³⁻⁵. Mycobacterium tuberculosis and related bacteria in the Mycobacterium tuberculosis complex (MTBc) emerged at least 11,000 years ago and have been coevolving with their hosts since^{6,7}. This history has resulted in a highly transmissible taxon of bacteria with longevity within their host and advanced methods of immune system evasion⁷.

Due to this coevolution, modern M. tuberculosis and members of the MTBc share numerous characteristics and are found in every known environment (except in the polar regions) along with members of the Non-Tuberculous Mycobacterium (NTM) group^{7,8}. The MTBc is made up of 10 Mycobacterium capable of causing TB or TB-like disease within their hosts, with the three specialized human TB species being Mycobacterium tuberculosis sensu stricto, Mycobacterium canettii and Mycobacterium africanum^{1,7,9}. Additionally, zoonotic TB transfer is well documented from cattle (Mycobacterium bovis), goats and sheep (Mycobacterium caprae), seals and sea lions (Mycobacterium pinnipedii), and rodents (Mycobacterium microti) into humans and vice versa^{4,6,7}. Recently, three more species have been added; Mycobacterium orygis in cattle and antelope^{7,10,} Mycobacterium suricattae in meerkats^{7,11}, and Mycobacterium mungi in mongeese^{7,12}.

Current research demonstrates MTBc members are highly genetically homogenous with up to 99.7% nucleotide identity and having identical 16S sequences⁷. MTBc members are primarily clonal with little horizontal gene transfer making differentiation between species difficult at the genetic level and impossible using microscopic methods^{2,4,6,13}.

Mycobacteria are gram-positive acid-fast bacilli approximately 2 μm long, which are primarily transmitted via aerosols; they are strictly intracellular, and do not have a known environmental reservoir outside of their endemic hosts^{1,7,14}. Lipid-rich cellular walls and layers of peptidoglycan, lipoglycan, mycolic acids, and waxes create an extremely hardy microbe^{7,14}. A defining characteristic of many mycobacteria, and all members of the MTBc, is fastidiousness and slow rate of growth in culture and in vivo^{2,6,15,16}.

Tuberculosis most commonly presents as a pulmonary disease (around 80% of cases), although extrapulmonary and disseminated disease presentations do also occur^{1,2,17}. Mycobacterial diseases cause a high burden of disease in low- and middle-income and developing countries (LMICs) around the world^{3,6,18}. It is estimated that one-third of the human population harbour latent TB (LTBI) and there are between nine and eleven million incident TB cases annually, according to the World Health Organization (WHO) 19. The number of annual fatalities attributed to TB has been estimated at 1.5-2 million deaths globally, making TB the greatest single threat for infection associated mortality^{6,20,2}.

### Mycobacterial Drug Resistance

The WHO defines drug resistance as a microorganism's resistance to an antimicrobial drug that was once able to treat an infection by that microorganism. The emergence of drug resistant (DR) strains of TB is largely a result of inconsistent practice of treatment protocols, delayed treatment and/or patients defaulting on lengthy treatment courses, leading to positive selection for drug-resistance and a higher incidence of resistant strain transfer between hosts^{3,22,23}.

There are currently several types of drug-resistant TB: multidrug-resistant (MDR) which is resistant to at least rifampicin and isoniazid; extensively drug-resistant (XDR) which has added resistances to any fluoroquinolone and at least one second-line injectable medication beyond what is found in MDR; extremely drug-resistant (XXDR) which is resistant to all first- and second-line medications; and totally drug-resistant (TDR) which has resistance to all current TB medications^{16,24}. Additionally, some species within the MTBc have lineage specific inherent resistances, e.g. M. bovis and M. canettii, which if misdiagnosed can complicate resistance-control methods^{2,22,24}.

Drug-resistant TB (DR-TB) is a growing issue globally as it increases in incidence^{21,22,25}. Concerns are that drug-resistant strains will reverse the progress made towards TB eradication^{6,22,23}. The incidence of drug resistant-TB worldwide has increased at least 10-fold in the past decade, with only 4.9% of patients demonstrating drug resistance in 2009 compared to 51% in 2018¹⁹. In 2018 nearly 500,000 of approximately 10.5 million TB cases in the world were MDR and of those 31,000 (6.2%) were XDR¹⁹.

MDR-TB is the most common type of resistance^{16,24}. MDR is defined as a TB strain which is resistant to isoniazid and rifampicin²⁵. MDR-TB strains are typically treated with traditional WHO endorsed drug regimens which require a 6-month course of first- and second-line antibiotics. XDR-TB is an MDR strain with additional resistance to the second-line medications of any fluoroquinolones and amikacin, capreomycin, or kanamycin^{25,26}. The specific regimen chosen to treat XDR-TB can be guided by culture or molecular (e.g. GenoType MTBDRsl—Bruker) drug susceptibility testing (DST) assays^{6,26,27} where available. Due to difficulties in diagnosing and treating MDR and XDR strains of TB, the mortality rates in these cases are high with approximately 50% mortality MDR and over 70% in XDR-TB infections 25.

The first line treatment for TB is a combination of antibiotics; rifampicin, isoniazid, ethambutol, and pyrazinamide over 6 months. Resistance to these antibiotic therapies leads to the use of second-line antibiotics (fluoroquinolones, amikacin, capreomycin, and kanamycin), which are less effective and more toxic^{24,25}. These therapeutics often require injections which necessitate more advanced medical infrastructure and oversight for treatment²⁴.

Drug resistance in Mycobacteria is mutational, rather than transferrable, and numerous single nucleotide polymorphisms (SNPs) have been reported to be associated with drug-resistance over the past decades—however, not all have sufficient evidence in the literature to support this association. The World Health Organisation (WHO) and others have graded reported drug-resistance SNPs into high, moderate and low confidence brackets^{28,29}.

### Targeted Next-Generation Sequencing

The WHO has announced a goal to effectively eradicate TB by 2035 and released guidelines on how to achieve that goal in 201522,23,25,30. Central to the WHO defined eradication strategy was a call for new diagnostic technologies and more rapid drug-susceptibility testing (DST) capabilities^{23,30-32}. Further was the requirement that these technologies should be effective for use in high-incidence, low-resource countries where the TB burden is high and medical infrastructure is generally lacking^{6,21,30}.

The non-molecular ‘gold-standard’ for detection of MTb and investigation of antibiotic resistance is culturing of a sample from a patient. However, culturing requires trained lab technicians and is typically extremely slow. The current ‘gold-standard’ molecular assay for detection of MTb and investigation of rifampicin (RIF) resistance (a surrogate marker for MDR-TB) is the Xpert MTB/RIF assay, a cartridge-based nucleic acid amplification test which can give rapid results. This test is easy to use, however, it can only identify RIF resistance so cannot diagnose XDR-TB 33.

The FIND (Foundation for Innovative New Diagnostics) Seq&Treat programme (https://www.finddx.org/tb/seq-treat/) specifically called for the development of targeted next generation sequencing (tNGS) based tests for DR-TB that that could be evaluated by FIND and potentially endorsed by the WHO. Sequencing-based tests have the potential to detect all resistance associated SNPs, thereby determine which drugs will work best against the MTB strain infecting the patient (Kayomo et al. Sci Rep 10, 10786 (2020). https://doi.org/10.1038/s41598-020-67479-4).

tNGS allows sequencing of specific areas of the genome using next generation sequencing to detect variants within the regions of interest. There are different approaches to targeted sequencing, the most common being amplicon sequencing, which uses PCR primers to amplify the sequence/s of interest.

When multiple genes are to be targeted, multiplex polymerase chain reactions (multiplex PCRs) may be used to amplify several different DNA target sequences simultaneously. This process amplifies DNA in samples using multiple primers and a temperature-mediated DNA polymerase in a thermal cycler.

As drug-resistant SNPs are present at multiple sites across the genome, multiple regions need to be targeted by PCR. Multiplex PCR offers substantial advantages over amplification of single regions in separate reactions including higher throughput, cost savings (fewer deoxyribonucleotide triphosphates, enzymes, and other consumables required), turnaround time and production of more data from limited starting material.

Primer design for multiplexed PCR is, however, complex. The primers must have similar annealing temperatures, each pair needs to be specific for its target, and primer pairs should amplify similar sized PCR product to ensure similar amplification efficiency between the multiple targets in the reaction. In addition, interaction between primers in multiplex reactions can reduce efficiency of amplification and the more primers in a reaction, the more likely this will occur. Designing efficient, sensitive and specific multiplex PCRs, particularly for multiplex reactions involving more than 5 or 6 primer sets, is challenging, and success is not assured.

Deeplex® Myc-TB, developed by Genoscreen, is an example of a targeted DR-TB test for prediction of resistance to 15 anti-tuberculous drugs, based on Illumina short read sequencing 34,35 (other tests have been developed but all have similar sensitivity and turnaround time). This test takes approximately 2 days to perform and has a limit of detection of ˜1000 MTB cells. There remains a need for a more rapid and sensitive test.

PCT/GB2021/052121 discloses oligonucleotide primer sets for use in multiplex PCR wherein the sets of primers are grouped into multiplex groups, wherein the multiplex groups comprise forward and reverse primer pairs for amplifying a portion of (a) eis, embB, rrs, rv0678, and fabG1; (b) gyrA, rpoB, ethA, rplC, and katG; and/or (c) gidB, inhA, rrl, pncA, rpsL, and tlyA.

It is an aim of the present invention to provide a method for rapidly and accurately detecting and/or identifying the presence of drug resistant mutations in a sample from subjects with suspected or confirmed TB using tNGS. It is a further aim to develop primers for achieving this objective, with a focus on the development of primers for amplifying a portion of one or more of eis, embB, rrs, rv0678, fabG1, gyrA, rpoB, ethA, rplC, katG, gidB, inhA, rrl, pncA, rpsL, and tlyA. It is a further aim to develop an improved forward primer for use in amplifying a portion of inhA, which allows use of an inhA primer pair with one or more other primers for identifying drug resistant mutations in a sample from subjects with suspected or confirmed TB, and in particular with one or more primer pairs for amplifying a portion of eis, embB, rrs, rv0678, fabG1, gyrA, rpoB, ethA, rplC, katG, gidB, rrl, pncA, rpsL, and tlyA and further in particular, with a primer pair for amplifying a portion of fabG1. A further aim is the use of these primers in a single multiplex PCR reaction. It is a further aim of the present invention to provide an assay or kit comprising one or more sets of these primer pairs.

### SUMMARY

Single nucleotide polymorphisms (SNPs) known to confer resistance to first and second-line anti-TB drugs were selected, and primers developed for the selected targets and optimized for use in multiplex PCR. The gene targets were: eis, embB, rrs, rv0678, fabG1, gyrA, rpoB, ethA, rplC, katG, gidB, inhA, rrl, pncA, rpsL, tlyA.

Accordingly, in a first aspect there is provided an oligonucleotide for amplifying a portion of the gene inhA from M. tuberculosis and/or related bacteria in the M. tuberculosis complex, comprising or consisting of a forward primer specific for said portion, wherein the forward primer has a sequence as set out in: SEQ ID No. 23, SEQ ID No. 35, SEQ ID No. 36, SEQ ID No. 37 or SEQ ID No. 38.

In a second aspect there is provided an oligonucleotide primer set for amplifying a portion of the gene inhA from M. tuberculosis and/or related bacteria in the M. tuberculosis complex, wherein the set comprises or consists of a pair of forward and reverse primers specific for said portion, wherein the set comprises or consists of a forward primer according to the first aspect, and a reverse primer having a sequence as set out in SEQ ID No. 24.

In a third aspect there is provided one or more oligonucleotide primer sets for amplifying a portion of one or more genes from M. tuberculosis and/or related bacteria in the M. tuberculosis complex selected from the group comprising one or more of eis, embB, ethA, fabG1, gidB, gyrA, inhA, katG, pncA, rrl, rplC, rpoB, rpsL, rrs, rv0678 and tlyA, wherein each set comprises or consists of a pair of forward and reverse primers specific for said portion, wherein each primer has a sequence as set out in SEQ ID Nos. 1-32 or 35-38.

In some embodiments, the sets of oligonucleotide primers can be used for multiplex PCR. Sets of primers can thus be grouped into multiplex groups. In some embodiments, one or more multiplex groups can be formed. In some embodiments, the groups comprise at least two primer sets selected from: SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24. In some embodiments, the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24. In some embodiments, the group of oligonucleotide primer sets comprises each of the oligonucleotide primer sets set out in SEQ ID Nos. 1 to 32; or each of the oligonucleotide primer sets set out in SEQ ID Nos. 1 to 22, 24 to 32 and 35; or each of the oligonucleotide primer sets set out in of SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38. In some embodiments, the group comprises each of the oligonucleotide primer sets set out in SEQ ID Nos. 1 to 32.

In some embodiments according to the third aspect, the portion of the one or more genes to be amplified contains one or more mutations that confer antibiotic resistance to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones. In some such embodiments, the one or mutations are one or more single nucleotide polymorphisms.

In a fourth aspect there is provided a multiplex PCR reaction mixture comprising a group of oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inhA, katG, pncA, rrl, rplC, rpoB, rpsL, rrs, rv0678, tlyA, wherein each set comprises a pair of forward and reverse primers specific for said portion, wherein the group of oligonucleotide primer sets comprises at least two primer sets selected from SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24. In some embodiments, the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24. In some such embodiments, the multiplex PCR reaction mixture comprises each of the oligonucleotide primer sets set out in SEQ ID Nos. 1 to 32; or each of the oligonucleotide primer sets set out in SEQ ID Nos. 1 to 22, 24 to 32 and 35; or each of the oligonucleotide primer sets set out in SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38. In some embodiments, the multiplex PCR reaction mixture comprises each of the oligonucleotide primer sets set out in SEQ ID Nos. 1 to 32.

The multiplex PCR reaction mixture may comprise further ingredients and reagents required to perform multiplex PCR, such as buffers, deoxynucleotide triphosphates (dNTPs), DMSO, water and DNA polymerase.

In some multiplex embodiments, said primers may be mixed to a working concentration of about 0.2 to about 0.4 μM. In some embodiments, the primers may be mixed to a working concentration of about 0.2 μM, optionally with the exception of tlyA which in some embodiments may be mixed to a working concentration of about 0.3 μM for consistent target amplification. In some embodiments, the inhA primer may be mixed to a working concentration of about 0.4 μM.

In some multiplex embodiments, DMSO may be added to the PCR reaction mixture at a concentration of between around 0.5 and 4%, between around 1 and 3%, or preferably around 2%.

In some embodiments, the portion of the one or more genes from M. tuberculosis and/or related bacteria in the M. tuberculosis complex is obtained from a sample from a subject suspected or confirmed to have TB. The sample may be one or more tissues and/or bodily fluids obtained from the subject, including one or more of sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB. Typically, the sample includes cells and/or DNA from M. tuberculosis and/or related bacteria in the M. tuberculosis complex.

In a fifth aspect there is provided a method of detecting the presence of one or more mutations that confer antibiotic resistance in a sample comprising DNA from Mycobacterium tuberculosis and/or related bacteria in the M. tuberculosis complex, said method including the steps of:
(a) isolating or extracting DNA from the sample;
(b) amplifying relevant gene regions or amplicons by polymerase chain reaction;
(c) subjecting the amplified gene regions or amplicons to DNA sequencing; and
(d) detecting one or more mutations;
   wherein amplification step (b) is carried out using one or more oligonucleotide primer sets for amplifying a portion of one or more genes from M. tuberculosis and/or related bacteria in the M. tuberculosis complex selected from the group comprising one or more of eis, embB, ethA, fabG1, gidB, gyrA, inhA, katG, pncA, rrl, rplC, rpoB, rpsL, rrs, rv0678 and tlyA, wherein each set comprises or consists of a pair of forward and reverse primers specific for said portion, wherein each primer has a sequence as set out in SEQ ID Nos. 1-32 or 35-38.

Detection of a mutation is indicative of antibiotic resistance. Identification of the mutation informs or allows identification of the nature of the antibiotic resistance (i.e. the antibiotic to which the bacteria is resistant).

Accordingly, in a sixth aspect, there is provided a method of predicting whether a patient suffering from tuberculosis will respond to treatment with one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones, said method comprising a step of determining the presence of one or more drug resistant mutations in one or more genes selected from the group comprising one or more of eis, embB, ethA, fabG1, gidB, gyrA, inhA, katG, pncA, rrl, rplC, rpoB, rpsL, rrs, rv0678 and tlyA in DNA obtained from a sample from the patient, the method comprising:
(a) isolating or extracting DNA from the sample;
(b) amplifying relevant gene regions or amplicons by polymerase chain reaction;
(c) subjecting the amplified gene regions or amplicons to DNA sequencing; and
(d) detecting the one or more mutations;
   wherein amplification step (b) is carried out using one or more oligonucleotide primer sets for amplifying a portion of one or more of eis, embB, ethA, fabG1, gidB, gyrA, inhA, katG, pncA, rrl, rplC, rpoB, rpsL, rrs, rv0678 and tlyA, wherein each set comprises or consists of a pair of forward and reverse primers specific for said portion, wherein each primer has a sequence as set out in SEQ ID Nos. 1-32 or 35-38.

In some embodiments according to the fifth or sixth aspect, step (b) of the method is a multiplex PCR reaction using one or more groups of oligonucleotide primer sets, wherein the groups comprise at least two primer sets selected from: SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24. In some embodiments, the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24. In some embodiments, the group of oligonucleotide primer sets comprises each of the oligonucleotide primer sets set out in SEQ ID Nos. 1 to 32; or each of the oligonucleotide primer sets set out in SEQ ID Nos. 1 to 22, 24 to 32 and 35; or each the oligonucleotide primer sets set out in of SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38. In some embodiments, the group of oligonucleotide primer sets comprises each of the oligonucleotide primer sets set out in SEQ ID Nos. 1 to 32.

In some embodiments according to the fifth or sixth aspect, the mutations are within one or more genes selected from the group consisting of one or more of eis, embB, ethA, fabG1, gidB, gyrA, inhA, katG, pncA, rrl, rplC, rpoB, rpsL, rrs, rv0678 and tlyA.

In some embodiments the mutations are one or more single nucleotide polymorphisms.

In some embodiments, the antibiotic resistance is to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones.

In some embodiments according to the fifth or sixth aspect, detection of: (i) a mutation in embB using an oligonucleotide primer set comprising SEQ ID Nos. 3 and 4 indicates resistance to ethambutol; (ii) a mutation in fabG1 using an oligonucleotide primer set comprising SEQ ID Nos. 9 and 10; a mutation in inhA using an oligonucleotide primer set comprising SEQ ID Nos. 23 and 24; SEQ ID Nos. 35 and 24; SEQ ID Nos. 36 and 24; SEQ ID Nos. 37 and 24 or SEQ ID Nos. 38 and 24; and/or a mutation in katG using an oligonucleotide primer set comprising SEQ ID Nos. 19 and 20 indicates resistance to isoniazid; (iii) a mutation in pncA using an oligonucleotide primer set comprising SEQ ID Nos. 27 and 28 indicates resistance to pyrazinamide; (iv) a mutation in rpoB using an oligonucleotide primer set comprising SEQ ID Nos. 13 and 14 indicates resistance to rifampicin; (v) a mutation in gidB using an oligonucleotide primer set comprising SEQ ID Nos. 21 and 22; a mutation in rpsL using an oligonucleotide primer set comprising SEQ ID Nos. 29 and 30; and/or a mutation in rrs using an oligonucleotide primer set comprising SEQ ID Nos. 5 and 6 indicates resistance to streptomycin; (vi) a mutation in rrs using an oligonucleotide primer set comprising SEQ ID Nos. 5 and 6 indicates resistance to amikacin; (vii) a mutation in rv0678 using an oligonucleotide primer set comprising SEQ ID Nos. 7 and 8 indicates resistance to bedaquiline and/or clofazimine; (viii) a mutation in gidB using an oligonucleotide primer set comprising SEQ ID Nos. 21 and 22; a mutation in rrs using an oligonucleotide primer set comprising SEQ ID Nos. 5 and 6; and/or a mutation in tlyA using an oligonucleotide primer set comprising SEQ ID Nos. 31 and 32 indicates resistance to capreomycin; (ix) a mutation in gyrA using an oligonucleotide primer set comprising SEQ ID Nos. 11 and 12 indicates resistance to ciprofloxacin; (x) a mutation in ethA using an oligonucleotide primer set comprising SEQ ID Nos 15 and 16; a mutation in fabG1 using an oligonucleotide primer set comprising SEQ ID Nos. 9 and 10, and/or a mutation in inhA using an oligonucleotide primer set comprising SEQ ID Nos. 23 and 24; SEQ ID Nos. 35 and 24; SEQ ID Nos. 36 and 24; SEQ ID Nos. 37 and 24 or SEQ ID Nos. 38 and 24 indicates resistance to ethionamide; (xi) a mutation in eis using an oligonucleotide primer set comprising SEQ ID Nos. 1 and 2 and/or a mutation in rrs using an oligonucleotide primer set comprising SEQ ID Nos. 5 and 6 indicates resistance to kanamycin; (xii) a mutation in rplC using an oligonucleotide primer set comprising SEQ ID Nos. 17 and 18 indicates resistance to linezoild; (xiii) a mutation in gyrA using an oligonucleotide primer set comprising SEQ ID Nos. 11 and 12 indicates resistance to moxifloxacin, ofloxacin and/or quinolones.

In some embodiments according to the fifth or sixth aspect involving multiplex PCR, the oligonucleotide primers may be mixed to a working concentration of about 0.2 to about 0.4 μM. In some embodiments, the primers may be mixed to a working concentration of about 0.2 μM, optionally with the exception of tlyA which in some embodiments may be mixed to a working concentration of about 0.3 μM for consistent target amplification. In some embodiments, the inhA primer may be mixed to a working concentration of about 0.4 μM.

In some embodiments according to the fifth or sixth aspect, the DNA is from M. tuberculosis.

In some embodiments according to the fifth or sixth aspect, the sample is a clinical sample. The sample may be one or more tissues and/or bodily fluids obtained from a subjected suspected of having or confirmed to have TB, including one or more of sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB. Typically, the sample includes cells and/or DNA from M. tuberculosis and/or related bacteria in the M. tuberculosis complex. In some embodiments, the sample is a sputum sample from a subject suspected or confirmed to have TB.

In some embodiments, the samples undergo mechanical disruption in order to disrupt the cells in the sample and achieve cell lysis. Any suitable means may be used, for example bead beating.

The step of isolating or extracting DNA from the sample may be carried out by any suitable means, including by the use of an appropriate kit, using given or standard protocols. For example, a Maxwell RSC PureFood Pathogen Kit from Promega AS1660, with instructions for use. In some embodiments, a Maxwell RSC PureFood Pathogen Kit from Promega AS1660 may be used. In some such embodiments, the following modifications were made from the kit instructions: The kit teaches use of a 800 μl sample; in some embodiments, a 400 μl sample after bead beating was used. The kit teaches adding 200 μl lysis buffer A and incubating at 56° C. for 4 min with shaking; in some embodiments, 200 μl lysis buffer A was added together with 40 μl Proteinase k, with incubation at 65° C. for 10 min. The kit teaches addition of 300 μl of lysis buffer and then placing the sample on the robot; in some embodiments, 300 μl lysis buffer was added together with 400 μl PBS and the sample was then placed on the robot.

In embodiments according to the fifth or sixth aspect wherein more than one group of primer sets are used for the amplification step, each group may be run as a separate or single multiplex group template.

Labelled nucleotides or labelled primers may be used in the amplification of the DNA for the purpose of, for example, quality control. For example, a fluorescent DNA-binding dye may be added to enable DNA quantitation. Any suitable dyes or probes with dyes may be used, such as probes with fluorescent dyes, such as use of a sybr green assay such as Roche Lightcycler® 480 SYBR Green I master.

In embodiments wherein more than one group of primer sets are used for the amplification step and each group is run as a separate multiplex group template, one or more multiplex group templates may be pooled to make a single template for DNA quantitation and/or sequencing.

Samples may then undergo barcode ligation and adaptor ligation to create a library for sequencing. Barcoding can be used when the amount of data required per sample is less than the total amount of data that can be generated: it allows pooling of multiple samples and sequencing of them together. Any suitable means may be used, including the use of barcoding kits, using given or standard protocols. For example, Oxford Nanopore Technologies provides amplicon barcoding with native barcoding expansion 96 (EXP-NBD196 and SQK-LSK109), including instructions for use. In some embodiments, the Oxford Nanopore Technologies amplicon barcoding with native barcoding expansion 96 (EXP-NBD196 and SQK-LSK109) may be used following the instructions for use provided.

The DNA sequencing step may be carried out by any suitable means. In preferred embodiments, the DNA sequencing is tNGS or third-generation sequencing (also known as long-read sequencing). Third-generation sequencing may be carried out using Oxford Nanopore Technologies' MinION, or PacBio's sequencing platform of single molecule real time sequencing (SMRT). Oxford Nanopore's sequencing technology is based on detecting the changes in electrical current passing through a nanopore as a piece of DNA moves through the pore. The current measurably changes as the bases G, A, T and C pass through the pore in different combinations. SMRT is based on the properties of zero-mode waveguides. Signals in the form of fluorescent light emission from each nucleotide are incorporated by a DNA polymerase bound to the bottom of the zL well. In preferred embodiments the sequencing is long-read nanopore sequencing.

The step of detecting of one or more mutations may be carried out by any suitable method, such as suitable bioinformatics tools and programmes. In some embodiments, the Oxford Nanopore Technologies workflow for TB may be used in desktop program EPI2ME with the FASTQ TB RESISTANCE PROFILE v2020.03.11.

The oligonucleotide primer sets and oligonucleotide primer set groups of the second and third aspects, the PCR reaction mixture of the fourth aspect and/or the methods of the fifth or sixth aspects can be used to identify both the presence and identity of drug resistance mutations in the genes of TB bacteria from a particular subject. Such information informs decisions regarding drug administration and allows a tailored treatment regime to be determined for the patient depending upon the identified mutations.

As such, in a seventh aspect, there is provided a method for determining an appropriate antibiotic treatment regime for a patient with tuberculosis, comprising detecting the presence of one or more mutations that confer antibiotic resistance in a sample from the patient according to the fifth aspect, and determining an appropriate antibiotic regime on the basis of the mutations detected/identified. The disclosure herein also provides a method of assigning a patient with tuberculosis to one of a certain number of treatment pathways comprising detecting and/or identifying the presence of one or more mutations that confer antibiotic resistance in a sample from the patient using a method according to the fifth aspect, and assigning the patient to a treatment regime on the basis of the mutations detected/identified.

In an eighth aspect there is provided a kit comprising one or more oligonucleotide primer sets for amplifying a portion of one or more genes from M. tuberculosis and/or related bacteria in the M. tuberculosis complex selected from the group comprising one or more of eis, embB, ethA, fabG1, gidB, gyrA, inhA, katG, pncA, rrl, rplC, rpoB, rpsL, rrs, rv0678 and tlyA, wherein each set comprises or consists of a pair of forward and reverse primers specific for said portion, wherein each primer has a sequence as set out in SEQ ID Nos. 1-32 or 35-38. In some embodiments the kit comprises at least two primer sets selected from: SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24. In some such embodiments, the oligonucleotide primer sets comprise at least SEQ ID Nos. 23 and 24; SEQ ID Nos. 35 and 24; SEQ ID Nos. 36 and 24; SEQ ID Nos. 37 and 24; or SEQ ID Nos. 38 and 24. In some embodiments, the kit comprises each of the oligonucleotide primer sets set out in SEQ ID Nos. 1 to 32; or each of the oligonucleotide primer sets set out in SEQ ID Nos. 1 to 22, 24 to 32 and 35; or each the oligonucleotide primer sets set out in of SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38. In some embodiments, the kit comprises each of the oligonucleotide primer sets set out in SEQ ID Nos. 1 to 32.

The kit may be used to carry out a method according to one or more of steps (a) (b) or (c) of the fifth aspect. The kit may further comprise ingredients and reagents required to carry out the method according to one or more of steps (a) (b) or (c) of the fifth aspect, including buffers, DNA polymerase and nucleotides. In some embodiments, the kit further comprises reagents required for the amplification of the gene regions between the primers. The kit may further comprise a sample collection container for receiving the sample. Samples may be processed according to the method of the fifth aspect immediately, alternatively they may be stored at low temperatures, for example in a fridge or freezer before the method is carried out. The sample may be processed before the method is carried out. For instance, a sedimentation assay may be carried out, and/or a preservative and/or dilutant may be added. Thus, the sample collection container may contain suitable processing solutions, such as buffers, preservative and dilutants.

Gene targets and their corresponding primer pairs according to the disclosure herein are as shown in Table 1.

TABLE 1

| Gene | Forward Primer | Reverse Primer |
|---|---|---|
| Target | (5′-3′) | (5′-3′) |
| eis | TGTCGGGTACCTTTCGAGC | TCCATGTACAGCGCCATCC |
| | SEQ ID. No. 1 | SEQ ID. No. 2 |
| embB | CGCCGTGGTGATATTCGGC | GCACACCGTAGCTGGAGAC |
| | SEQ ID. No. 3 | SEQ ID. No. 4 |
| rrs | CTCTGGGCAGTAACTGACGC | GAGTGTTGCCTCAGGACCC |
| | SEQ ID. No. 5 | SEQ ID. No. 6 |
| rv0678 | GCTCGTCCTTCACTTCGCC | ATCAGTCGTCCTCTCCGGT |
| | SEQ ID. No. 7 | SEQ ID. No. 8 |
| fabG1 | CTTTTGCACGCAATTGCGC | AGCAGTCCTGTCATGTGCG |
| | SEQ ID. No. 9 | SEQ ID. No. 10 |
| gyrA | TGACAGACACGACGTTGCC | CGATCGCTAGCATGTTGGC |
| | SEQ ID. No. 11 | SEQ ID. No. 12 |
| rpoB | TCATCATCAACGGGACCGAG | ACACGATCTCGTCGCTAACC |
| | SEQ ID. No. 13 | SEQ ID. No. 14 |
| ethA | TGGATCCATGACCGAGCAC | GTCCAGGAGGCATTGGTGT |
| | SEQ ID. No. 15 | SEQ ID. No. 16 |
| rplC | AGTACAAGGACTCGCGGGA | TCGAGTGGGTACCCTGGC |
| | SEQ ID. No. 17 | SEQ ID. No. 18 |
| katG | CTGTGGCCGGTCAAGAAGA | GGATCTGGCTCTTAAGGCTGG |
| redesigned | SEQ ID. No. 19 | SEQ ID. No. 20 |
| gidB | TGACACAGACCTCACGAGC | GCCCTTCTGATTCGCGATG |
| | SEQ ID. No. 21 | SEQ ID. No. 22 |
| inhA | CGGATTCTGGTTAGCGGAATCA | GGCGTAGATGATGTCACCC |
| redesigned | SEQ ID. No. 23 | SEQ ID. No. 24 |
| inhA FW 6 | | |
| rrl | GGTCCGTGCGAAGTCGC | TGAACCCGTGTTCTGCGG |
| | SEQ ID. No. 25 | SEQ ID. No. 26 |
| pncA | TCACCGGACGGATTTGTCG | TCCAGATCGCGATGGAACG |
| | SEQ ID. No. 27 | SEQ ID. No. 28 |
| rpsL | GCGGCGGGTATTGTGGTT | TAACCGGCGCTTCTCACC |
| | SEQ ID. No. 29 | SEQ ID. No. 30 |
| thyA | CGTTGATGCGCAGCGATC | GGTCTCGGTGGCTTCGTC |
| | SEQ ID. No. 31 | SEQ ID. No. 32 |
| katG initial | CTGTGGCCGGTCAAGAAGA | TGCCCGGATCTGGCTCTTA |
| | SEQ ID. No. 19 | SEQ ID. No. 33 |
| inhA initial | GGGCGCTGCAATTTATCCC | GGCGTAGATGATGTCACCC |
| | SEQ ID. No. 34 | SEQ ID. No. 24 |
| inhA redesigned | ACGGCAAACGGATTCTGGTT | GGCGTAGATGATGTCACCC |
| inhA FW 2 | SEQ ID. No. 35 | SEQ ID. No. 24 |
| inhA redesigned | TTCTGGTTAGCGGAATCATCACC | GGCGTAGATGATGTCACCC |
| inhA FW 8 | SEQ ID. No. 36 | SEQ ID. No. 24 |
| inhA redesigned | CTGGTTAGCGGAATCATCACCG | GGCGTAGATGATGTCACCC |
| inhA FW 9 | SEQ ID. No. 37 | SEQ ID. No. 24 |
| inhA redesigned | TTAGCGGAATCATCACCGACT | GGCGTAGATGATGTCACCC |
| inhA FW 11 | SEQ ID. No. 38 | SEQ ID. No. 24 |

### BRIEF DESCRIPTION OF FIGURES

For a better understanding of the invention, and to show how embodiments of the same may be carried into effect, reference will now be made, by way of example, to the accompanying Figures in which:

FIG. 1: qPCR curves showing nested qPCR amplification of multiplexed primers;

FIG. 2: Fragment size analysis of amplicons produced during each triplex reaction. A1—ladder, B1—triplex 1, C1—triplex 2, D1—triplex 3, E1—triplex 4 and F1—triplex 5;

FIG. 3: Example of nested qPCR results testing the amplification efficiency of individual gene targets within multiplex version 4, group 1;

FIG. 4: TapeStation imaging of 5-plex PCR products;

FIG. 5: Nested qPCR results for gene targets in multiplex group formulation 7;

FIG. 6: Nested qPCR results for gene targets in Multiplex group formulation 9, Group 2;

FIGS. 7A and 7B: Examples of even target coverage using redesigned inhA forward primer 2 (inhA FW 2) (a); and redesigned inhA forward primer 8 (inhA FW 8) (b); tested in each case with 104 M. tuberculosis genome equivalents;

FIGS. 8A and 8B: Target coverage using inhA redesigned forward primer 6 (inhA FW 6) at 100 copies (a); and 10 copies (b);

FIGS. 9A-9F: Target coverage and percentage mapped reads using redesigned inhA forward primer 6 (inhA FW 6) at 2× primer concentration only (a) compared with optimised conditions (2× primer concentration with 2% DMSO) (b); at 100 copies (FIG. 9A), 50 copies (FIG. 9B) and 10 copies (FIG. 9C).

### DETAILED DESCRIPTION

### Detectable Drug-Resistance SNPs

Selected target single nucleotide polymorphisms (SNPs) that confer resistance to first and second-line anti-TB drugs were chosen primarily from WHO/FIND evidence published in the WHO next-generation sequencing technical guide³⁶. The targets for rpsL were selected from prior literature by Karimi, et al. and Meier, et al^{37,38}. Targets for gidB were selected on evidence from Villellas, et al³⁹. Targets for ethA were selected on evidence from Morlock, et al⁴⁰. Targets for embB were selected on evidence from Zhao, et al⁴¹. Finally, targets for tlyA were selected from prior literature by Maus, et al⁴².

Base positions and genes as listed are based on the H37Rv M. tuberculosis reference genome available through the NCBI database (NC_000962.3)⁴³. Targeted mutations were identified either as their codon location or their nucleotide location. Mutations were identified by the codon which they effect when the SNP occurs within an annotated gene region and the prior literature explicitly states the altered amino acid. Targets were listed by nucleotide mutation in the event they occur within a gene promoter region or the supporting literature does not explicitly identify the amino acid mutation. These promoter region SNPs are further identified by a “-” prior to its position indicating it occurs before the annotated gene. The effect of the mutated base is also included; e.g. Asparagine to Histidine or nucleotide A to nucleotide C (Table A, appended).

### Multiplex Group Optimisation

Primers were developed for the chosen gene/promotor targets (n=16; Table 2) that amplified ˜1000 bp regions containing the targeted SNPs of interest. As discussed above, interaction between primers in multiplex reactions can reduce efficiency of amplification and the more primers in a reaction, the more likely this will occur. Therefore designing efficient, sensitive and specific multiplex PCRs is complex.

TABLE 2
Details of genes conferring drug resistance

| Drug | Genes conferring resistance |
|---|---|
| Ethambutol | embB |
| Isoniazid | fabG1 |
| | inhA |
| | katG |
| Pyrazinamide | pncA |
| Rifampicin | rpoB |
| Streptomycin | gidB |
| | rpsL |
| | rrs |
| Amikacin | rrs |
| Bedaquiline | rv0678 |
| Capreomycin | gidB |
| | rrs |
| | tlyA |
| Ciprofloxacin | gyrA |
| Clofazimine | rv0678 |
| Ethionamide | ethA |
| | fabG1 |
| | inhA |
| Kanamycin | eis |
| | rrs |
| Linezolid | rplC |
| Moxifloxacin | gyrA |
| Ofloxacin | gyrA |
| Quinolones | gyrA |

The following genes were targeted in the DR-TB sequencing assay: eis, embB, ethA, fabG1, gidB, gyrA, inhA, katG, pncA, rrl, rplC, rpoB, rpsL, rrs, rv0678, tlyA. Initially, gene target primer pairs were grouped into 5 sets of three (Table 3). DNA was extracted from M. bovis BCG and used to test the specificity and sensitivity of the triplex assays.

TABLE 3
Gene targets per triplex

| | Gene Target 1 | Gene Target 2 | Gene Target 3 |
|---|---|---|---|
| Triplex 1 | Eis | ethA | embB |
| Triplex 2 | pncA | gyrA | rpoB |
| Triplex 3 | fabG1/inhA | rrs | gidB |
| Triplex 4 | rv0678 | rplC | katG |
| Triplex 5 | tlyA | rpsL | rrl |

The multiplex PCRs were performed as follows:

### Per Reaction:

5 μl DNA (concentration approx. 20 ng)
25 μl Qiagen 2× Multiplex Master Mix
10 μL Qiagen 5× Q-Solution
2.5 μl (10 μM, final conc 0.2 μM) Forward Multiplex Primer
2.5 μl (10 μM, final conc 0.2 μM) Reverse Multiplex Primer
5 μl Molecular H₂O

### PCR Conditions:

Cycling Conditions

| Step | Temperature (C.) | Time (mm:ss) | # Cycles |
|---|---|---|---|
| Pre-Incubation | 95 | 20:00 | 1 |
| Amplification | 94 | 00:30 | 35 |
| | 60 | 01:30 | |
| | 72 | 01:30 | |
| Extension | 72 | 10:00 | 1 |
| Hold | 4 | ∞ | 1 |

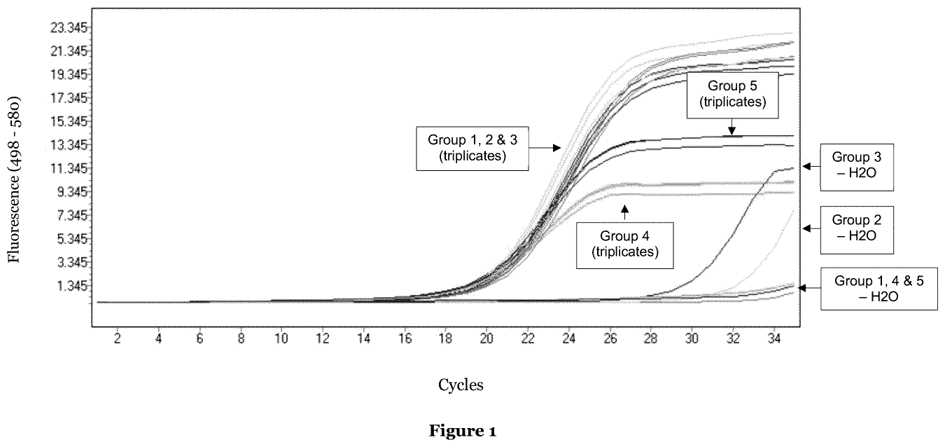

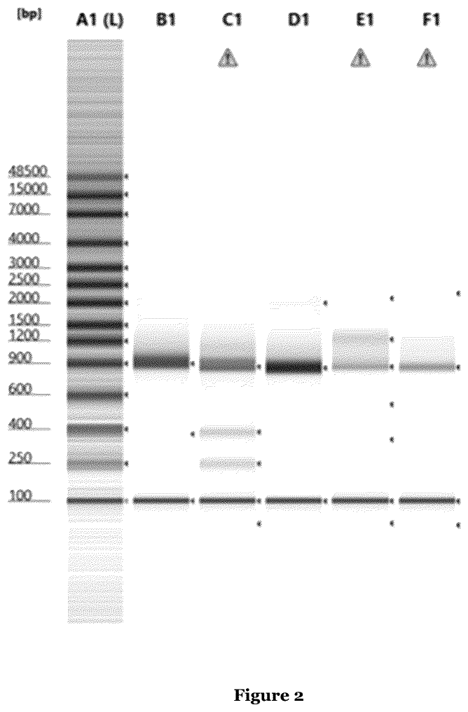

Nested qPCR was performed on the amplified products from the multiplex PCR to evaluate the amplification of all the targets. Nested PCR on all amplified products resulted in very similar Ct values, indicating the same amplification efficiency across all primers (FIG. 1). Fragment size analysis of the multiplex PCR amplicons expected at ˜1000 bp showed minimal non-specific amplification with additional amplicon bands only seen in Triplex 2 and Triplex 5 (FIG. 2: A1—ladder, B1—triplex 1, C1—triplex 2, D1—triplex 3, E1—triplex 4 and F1—triplex 5).

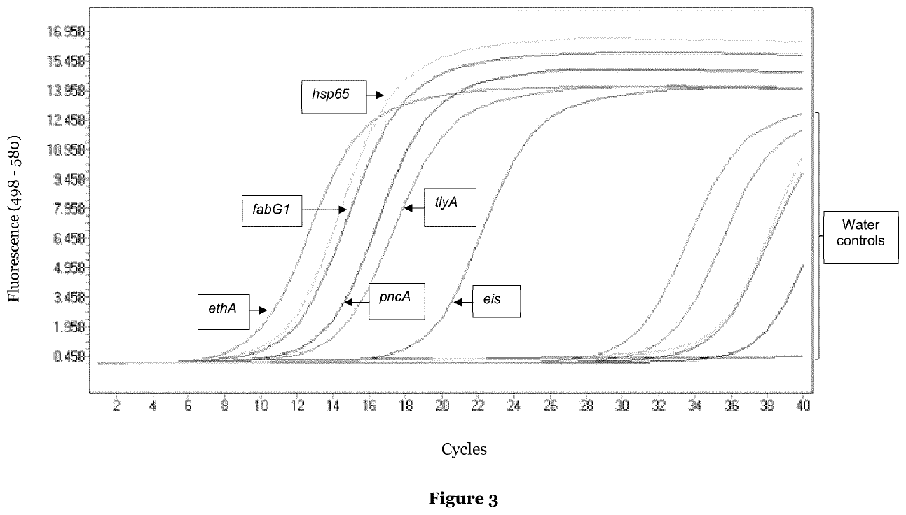

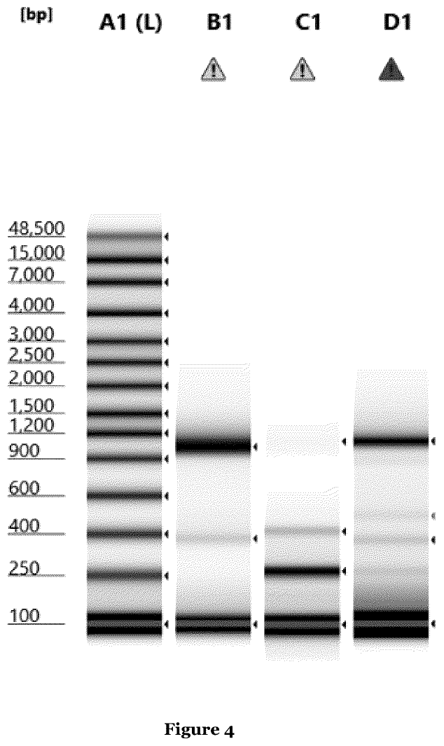

While the triplex assays worked well, the requirement for 5 PCR reactions was considered too laborious and expensive for the tNGS assay. Hence, the primer pairs were combined in a new format to make three groups (two 5-plex and one 6-plex reaction), in order to simplify the assay. Multiplex efficiency was again measured by nested qPCR (FIG. 3: Ct values range from 8-18 indicating inefficient amplification of some targets caused by primer interaction) and fragment size analysis was used to show any non-specific amplification (FIG. 4: Results show non-specific amplification in Group 2 (C1) with no visible band of expected size (˜1000 bp). Group 1 and Group 3 show less non-specific amplification but qPCR results showed inefficient amplification of some targets). Multiple multiplex primer combinations had to be tested as primer interaction led to amplification inefficiencies of one or more targets per multiplex. In total, nine different combinations were tested (Table 4). A new target for identifying Mycobacterium species, hsp65, was introduced at version 3. This was designed to provide more information in a case where a sample is negative for MTBC.

TABLE 4
The versions of the multiplex formulations tested during the optimisation process

| Multiplex Design Group | Group 1 Gene | Group 2 Gene | Group 3 Gene |
|---|---|---|---|
| Formulation Version | Targets | Targets | Targets |
| 1 | eis, ethA, embB, tlyA, | pncA, gyrA, rpoB, | fabG1, inhA, rrs, |
| | rv0678 | rpsL, rplC | gidB, rrl, katG |
| 2 | eis, ethA embB, tlyA, | gyrA, rpoB, rpsL, rplC, | fabG1, inhA, rrs, |
| | pncA | rv0678 | gidB, rrl, katG |
| 3 | eis, embB, eth.A, | gyrA, rpoB, fabG1, | inhA, rrs, gidB, rrl, |
| | pncA, tlyA, hsp65 | rpsL, rplC, rv0678 | katG |
| 4 | eis, ethA, pncA, tlyA, | gyrA, rpoB, rpsL, rplC, | inhA, rrs, gidB, rrl, |
| | hsp65, fabG1 | rv0678, embB | katG |
| 5 | ethA, pncA, hsp65, | gyrA, rpoB, rpsL, rplC, | inhA, gidB, rrl, |
| | rrs, embB | rv0678, fabG1 | katG, eis, tlyA |
| 6 | hsp65, rrs, rpsL, | gyrA, rpoB, rplC, | inhA, gidB, rrl, |
| | fabG1, tlyA | rv0678, ethA, embB | katG, eis, pncA |
| 7 | fabG1, rrs, rv0678, | gyrA, rpoB, rplC, ethA, | inhA, gidB, rrl, |
| | eis, embB | katG, hsp65 | pncA, rpsL, tlyA |
| 8 | fabG1, rs, rv0678, | gyrA, rpoB, rplC, | gidB, rrl, pncA, |
| | ethA, inhA | katG, hsp65, embB | rpsL, tlyA, eis |
| 9 | fabG1, rrs, rv0678, | gyrA, rpoB, rplC, | gidB, rrl, pncA, |
| | ethA, inhA | katG, embB | rpsL, tlyA, eis |

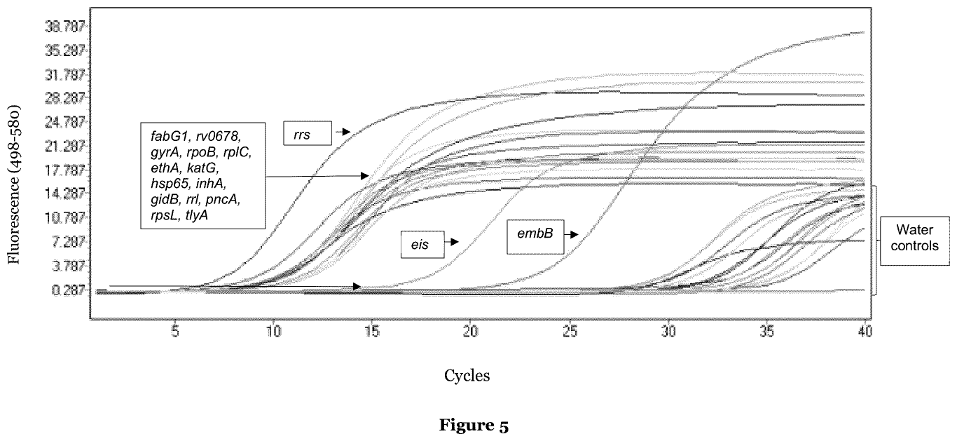

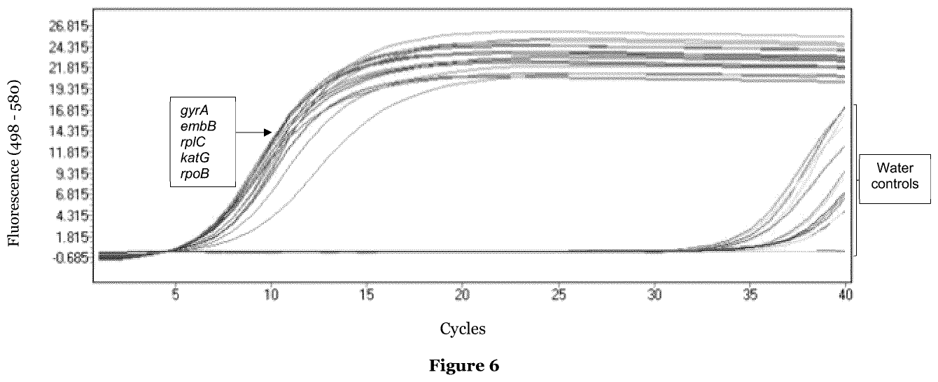

Formulations 1-6 had multiple late Cts and/or total dropouts indicative of inhibition and competition within the multiplex groups. Version 7 showed multiplex groups 2 and 3 had Ct ranges <1.5 while group 1 had a range of approximately 15 Cts (FIG. 5). Subsequent optimisations led to two more versions, resulting in the final version 9 which had all multiplex group Ct ranges <2 (FIG. 6).

### Final Primer Design

Concurrently to optimising the group formulations, various primers were redesigned to overcome primer interactions. In total there were 48 multiplex primer combinations with >300 primer designs (Table 5) before the optimal sequences were determined.

After testing ˜400 samples provided by FIND in a lab validation study (described below), a re-design was required for the katG reverse primer to avoid a common non-resistance conferring SNP in the primer binding site. To overcome this, five new reverse primers were tested where each primer was shifted towards the 3′ 1 bp at a time (up to 5 bp shift) (Table 6). Option 5 was selected for the final assay as the mutation site was avoided and the performance of the assay wasn't negatively affected.

TABLE 6
Redesigned katG primer options (non-resistance
conferring SNP in bold).

| Base Pair Positions | |
|---|---|
| Shifted Toward 3′ | Primer sequence (5′-3′) |
| Original Primer | TGCCCGGATCTGGCTCTTA |
| 1 | GCCCGGATCTGGCTCTTAA |
| 2 | CCCGGATCTGGCTCTTAAGG |
| 3 | CCGGATCTGGCTCTTAAGGC |
| 4 | CGGATCTGGCTCTTAAGGCTG |
| 5 | GGATCTGGCTCTTAAGGCTGG |

It was further desirable to combine the primer pairs in a single 16-plex reaction. Initial testing of all primers together identified an overlap of the inhA amplicon with the neighbouring fabG1 gene amplicon. This resulted in the forward inhA primer and the reverse fabG1 primer combining to generate a 175 bp amplicon, as shown below:

Sequence outside the annotated gene is highlighted in grey. fabG1 start and end gene codons plus primers are written in italics. inhA start and end gene codons plus primers are written in bold.

CTTTTGCACGCAATTGCGCGGTCAGTTCCACACCCTGCGGCACGTACACGTCTTTATG
TAGCGCGACATACCTGCTGCGCAATTCGTAGGGCGTCAATACACCCGCAGCCAGGGC
CTCGCTGCCCAGAAAGGGATCCGTCATGGTCGAAGTGTGCTGAGTCACACCGACAAA
CGTCACGAGCGTAACCCCAGTGCGAAAGTTCCCGCCGGAAATCGCAGCCACGTTACG
CTCGTGGACATACCGATTTCGGCCCGGCCGCGGCGAGACGATAGGTTGTCGGG
GTGACTGCCACAGCCACTGAAGGGGCCAAACCCCCATTCGTATCCCGTTCAGTCCTGG
TTACCGGAGGAAACCGGGGGATCGGGCTGGCGATCGCACAGCGGCTGGCTGCCGAC
GGCCACAAGGTGGCCGTCACCCACCGTGGATCCGGAGCGCCAAAGGGGCTGTTTGG
CGTCGAATGTGACGTCACCGACAGCGACGCCGTCGATCGCGCCTTCACGGCGGTAGA
AGAGCACCAGGGTCCGGTCGAGGTGCTGGTGTCCAACGCCGGCCTATCCGCGGACG
CATTCCTCATGCGGATGACCGAGGAAAAGTTCGAGAAGGTCATCAACGCCAACCTCA
CCGGGGCGTTCCGGGTGGCTCAACGGGCATCGCGCAGCATGCAGCGCAACAAATTC
GGTCGAATGATATTCATAGGTTCGGTCTCCGGCAGCTGGGGCATCGGCAACCAGGC
CAACTACGCAGCCTCCAAGGCCGGAGTGATTGGCATGGCCCGCTCGATCGCCCGCGA
GCTGTCGAAGGCAAACGTGACCGCGAATGTGGTGGCCCCGGGCTACATCGACACCG
ATATGACCCGCGCGCTGGATGAGCGGATTCAGCAGGGGGCGCTGCAATTTATCCCA
GCGAAGCGGGTCGGCACCCCCGCCGAGGTCGCCGGGGTGGTCAGCTTCCTGGCTTC
CGAGGATGCGAGCTATATCTCCGGTGCGGTCATCCCGGTCGACGGCGGCATGGGTA
TGGGCCACTGACACAACACAAGGACGCACATGACAGGACTGCTGGACGGCAAACGG
ATTCTGGTTAGCGGAATCATCACCGACTCGTCGATCGCGTTTCACATCGCACGGGTA
GCCCAGGAGCAGGGCGCCCAGCTGGTGCTCACCGGGTTCGACCGGCTGCGGCTGAT
TCAGCGCATCACCGACCGGCTGCCGGCAAAGGCCCCGCTGCTCGAACTCGACGTGCA
AAACGAGGAGCACCTGGCCAGCTTGGCCGGCCGGGTGACCGAGGCGATCGGGGCG
GGCAACAAGCTCGACGGGGTGGTGCATTCGATTGGGTTCATGCCGCAGACCGGGAT
GGGCATCAACCCGTTCTTCGACGCGCCCTACGCGGATGTGTCCAAGGGCATCCACAT
CTCGGCGTATTCGTATGCTTCGATGGCCAAGGCGCTGCTGCCGATCATGAACCCCGG
AGGTTCCATCGTCGGCATGGACTTCGACCCGAGCCGGGCGATGCCGGCCTACAACTG
GATGACGGTCGCCAAGAGCGCGTTGGAGTCGGTCAACAGGTTCGTGGCGCGCGAG
GCCGGCAAGTACGGTGTGCGTTCGAATCTCGTTGCCGCAGGCCCTATCCGGACGCTG
GCGATGAGTGCGATCGTCGGCGGTGCGCTCGGCGAGGAGGCCGGCGCCCAGATCC
AGCTGCTCGAGGAGGGCTGGGATCAGCGCGCTCCGATCGGCTGGAACATGAAGGAT
GCGACGCCGGTCGCCAAGACGGTGTGCGCGCTGCTGTCTGACTGGCTGCCGGCGAC
CACGGGTGACATCATCTACGCCGACGGCGGCGCGCACACCCAATTGCTCTAG

The inhA forward primer was redesigned to remove the overlap, by positioning it downstream of the fabG1 reverse primer. 12 inhA forward primers were designed, as shown in Table B, below.

TABLE B
Redesigned inhA forward primers

| Primer name | Primer sequence (5′-3′) |
|---|---|
| inhA_FW_1 | GGACGGCAAACGGATTCTG |
| inhA_FW_2 | ACGGCAAACGGATTCTGGTT |
| inhA_FW_3 | GGCAAACGGATTCTGGTTAGC |
| inhA_FW_4 | CAAACGGATTCTGGTTAGCGG |
| inhA_FW_5 | AACGGATTCTGGTTAGCGGA |
| inhA_FW_6 | CGGATTCTGGTTAGCGGAATCA |
| inhA_FW_7 | GATTCTGGTTAGCGGAATCATCACC |
| inhA FW_8 | TTCTGGTTAGCGGAATCATCACC |
| inhA_FW_9 | CTGGTTAGCGGAATCATCACCG |
| inhA_FW_10 | GGTTAGCGGAATCATCACCG |
| inhA FW_11 | TTAGCGGAATCATCACCGACT |
| inhA_FW_12 | AGCGGAATCATCACCGACTC |

The redesigned inhA forward primers in Table B were each tested in a single multiplex reaction with the reverse inhA primer (SEQ ID NO: 24) and the other primer pairs (SEQ ID Nos: 1-22 and 25-32). Methodological details are provided in Example 3.

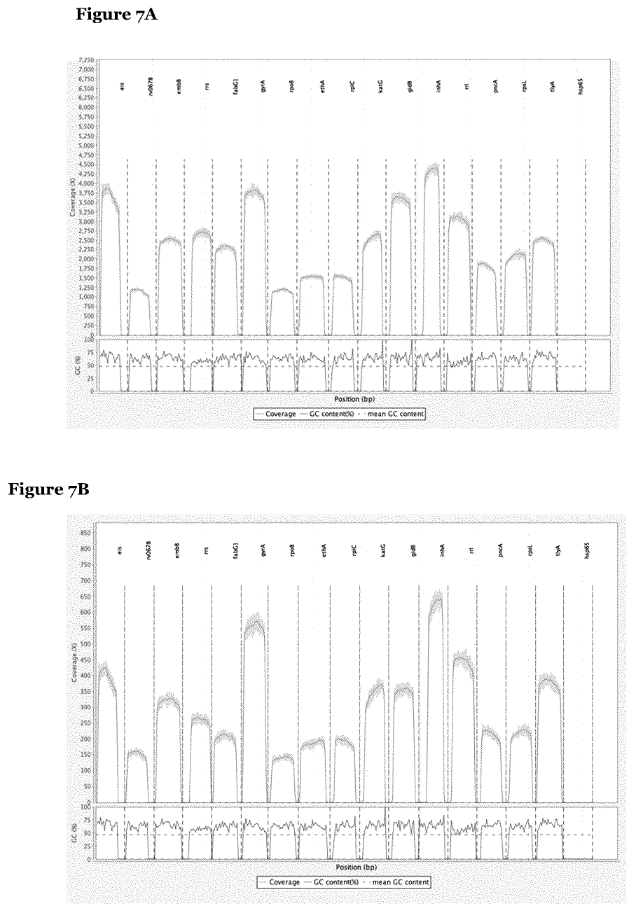

Five of the 12 redesigned inhA forward primers (inhA_FW 2, 6, 8 9 and 11, marked in bold in Table B) performed well in the single multiplex reaction when using high target concentration i.e. 104 M. tuberculosis genome equivalents, in each case resulting in relatively even coverage (˜5 fold coverage difference between lowest and highest for all 16 targets). FIG. 7 shows even target coverage using redesigned inhA forward primer 2 (inhA_FW 2) (a) and redesigned inhA forward primer 8 (inhA_FW 8) (b) tested with 104 M. tuberculosis genome equivalents. This was a surprising result, as a single primer change made it possible to combine all 16 primer pairs with good performance, something that has not, to date, been possible. The remaining redesigned primers resulted in various amplicon drop-outs, indicating primer interactions.

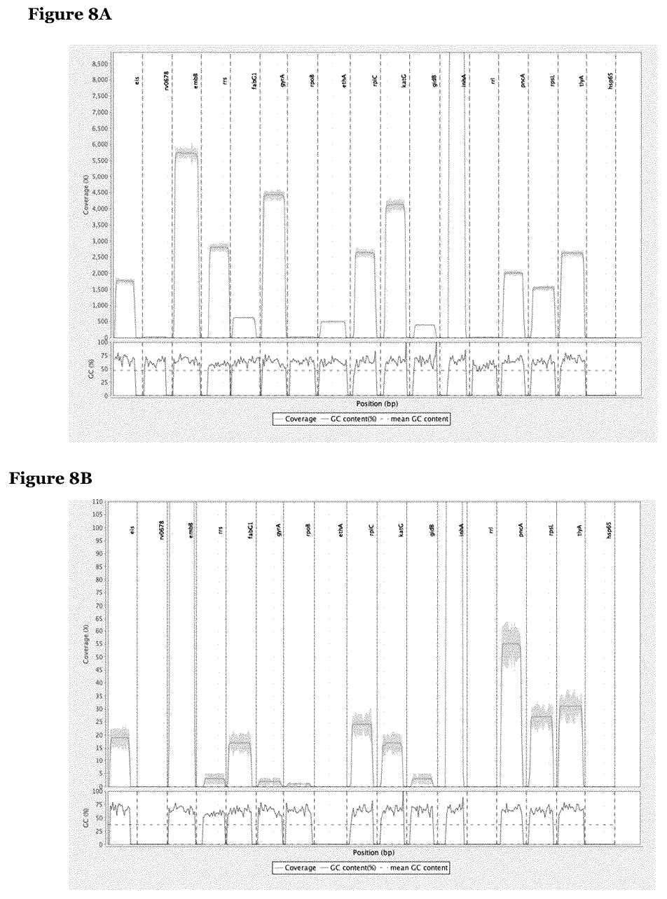

Samples containing lower M. tuberculosis concentrations (100 and 10 genome equivalents) were then tested with the 5 best performing inhA forward primers (inhA_FW 2, 6, 8, 9 and 11) to determine assay sensitivity. Each of inhA_FW 2, 6, 8, 9 and 11 performed well, though some target drop outs were observed at low target concentrations (see FIG. 8, which shows target coverage when using redesigned inhA_FW 6 at 100 copies (a) and 10 copies (b)).

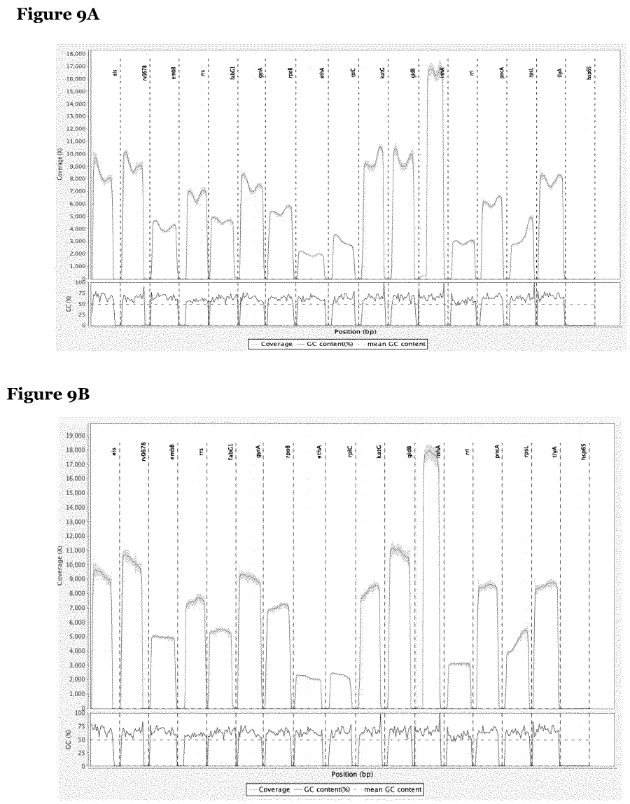

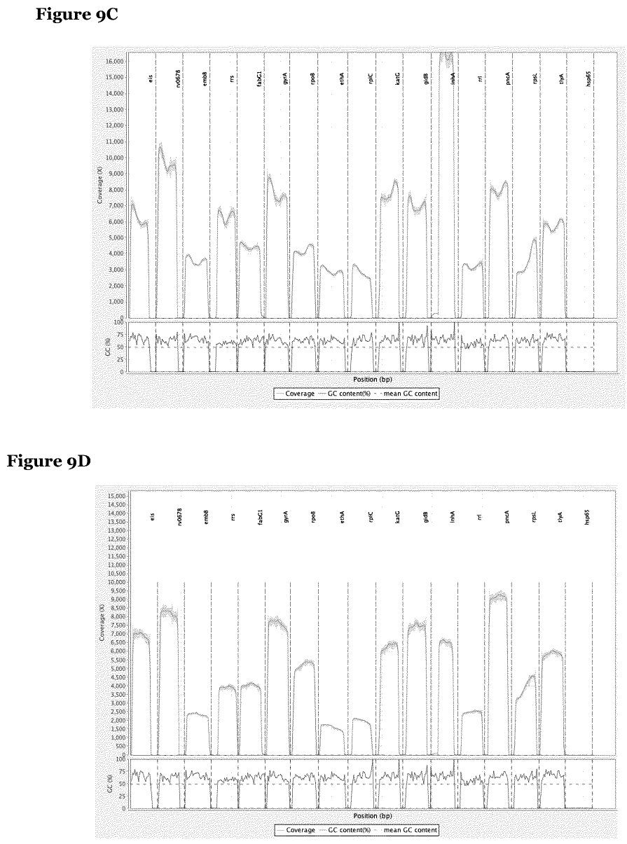

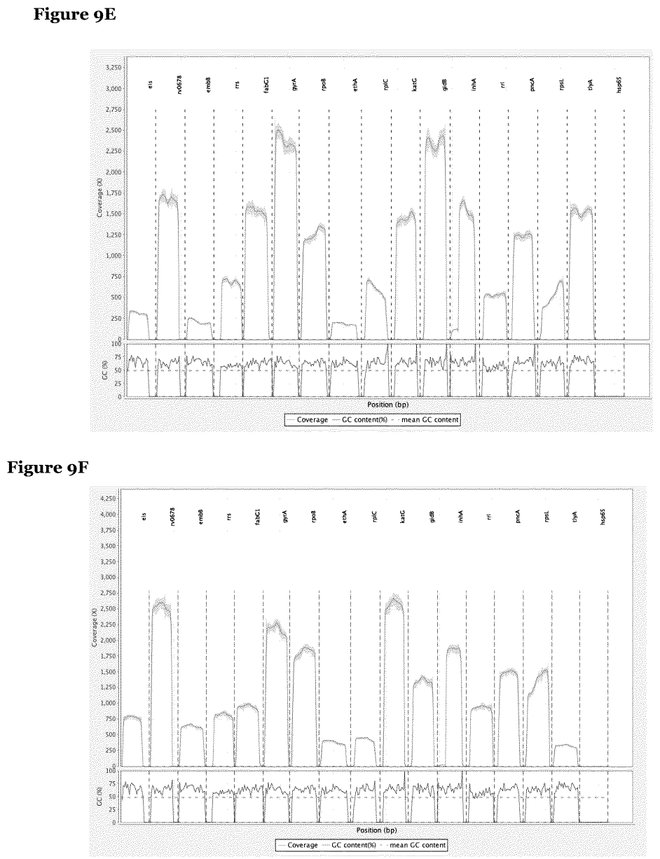

Optimisation was undertaken using, as an example, inhA_FW 6. Reaction conditions were optimised to improve evenness of coverage for the targets and thereby improve assay sensitivity. Different polymerases, MgCl₂ concentrations and annealing temperatures, primer balancing for low targets and the addition of DMSO were tested. It was found that combining 2× primer concentration (from 0.2 μM to 0.4 μM) with 2% DMSO resulted in best performance, improving evenness of coverage and the proportion of mapped reads at low target input. FIGS. 9A-C show target coverage and percentage mapped reads using the inhA_FW 6 primer, for 2× primer concentration only (a); compared with optimised conditions (2× primer concentration with 2% DMSO) (b). In FIG. 9A (100 copies), the 2× primer concentration only (a) mapped reads were 67% compared to the optimised conditions (b) which were 81%; in FIG. 9B (50 copies), the 2× primer concentration only (a) mapped reads were 62% compared to the optimised conditions (b) which were 81%; in FIG. 9C (10 copies), the 2× primer concentration only (a) mapped reads were 62% compared to the optimised conditions (b) which were 74%.

The final optimal iteration of primers for use in a single multiplex assay is provided in Table 7.

TABLE 7
Primer sequences

| Target and | | |
|---|---|---|
| Orientation | Sequence (5′-3′) | SEQ ID No |
| eis Forward | TGTCGGGTACCTTTCGAGC | SEQ ID No. 1 |
| eis Reverse | TCCATGTACAGCGCCATCC | SEQ ID No. 2 |
| embB Forward | CGCCGTGGTGATATTCGGC | SEQ ID No. 3 |
| embB Reverse | GCACACCGTAGCTGGAGAC | SEQ ID No. 4 |
| rrs Forward | CTCTGGGCAGTAACTGACGC | SEQ ID No. 5 |
| rrs Reverse | GAGTGTTGCCTCAGGACCC | SEQ ID No. 6 |
| rv0678 Forward | GCTCGTCCTTCACTTCGCC | SEQ ID No. 7 |
| rv0678 Reverse | ATCAGTCGTCCTCTCCGGT | SEQ ID No. 8 |
| fabG1 Forward | CTTTTGCACGCAATTGCGC | SEQ ID No. 9 |
| fabG1 Reverse | AGCAGTCCTGTCATGTGCG | SEQ ID No. 10 |
| gyrA Forward | TGACAGACACGACGTTGCC | SEQ ID No. 11 |
| gyrA Reverse | CGATCGCTAGCATGTTGGC | SEQ ID No. 12 |
| rpoB Forward | TCATCATCAACGGGACCGAG | SEQ ID No. 13 |
| rpoB Reverse | ACACGATCTCGTCGCTAACC | SEQ ID No. 14 |
| ethA Forward | TGGATCCATGACCGAGCAC | SEQ ID No. 15 |
| ethA Reverse | GTCCAGGAGGCATTGGTGT | SEQ ID No. 16 |
| rplC Forward | AGTACAAGGACTCGCGGGA | SEQ ID No. 17 |
| rplC Reverse | TCGAGTGGGTACCCTGGC | SEQ ID No. 18 |
| katG Forward | CTGTGGCCGGTCAAGAAGA | SEQ ID No. 19 |
| katG Reverse | GGATCTGGCTCTTAAGGCTGG | SEQ ID No. 20 |
| redesigned | | |
| gidB Forward | TGACACAGACCTCACGAGC | SEQ ID No. 21 |
| gidB Reverse | GCCCTTCTGATTCGCGATG | SEQ ID No. 22 |
| inhA Forward | CGGATTCTGGTTAGCGGAATCA | SEQ ID No. 23 |
| redesigned: | | |
| inhA FW 6 | | |
| inhA Reverse | GGCGTAGATGATGTCACCC | SEQ ID No. 24 |
| rrl Forward | GGTCCGTGCGAAGTCGC | SEQ ID No. 25 |
| rrl Reverse | TGAACCCGTGTTCTGCGG | SEQ ID No. 26 |
| pncA Forward | TCACCGGACGGATTTGTCG | SEQ ID No. 27 |
| pncA Reverse | TCCAGATCGCGATGGAACG | SEQ ID No. 28 |
| rpsL Forward | GCGGCGGGTATTGTGGTT | SEQ ID No. 29 |
| rpsL Reverse | TAACCGGCGCTTCTCACC | SEQ ID No. 30 |
| tlyA Forward | CGTTGATGCGCAGCGATC | SEQ ID No. 31 |
| tlyA Reverse | GGTCTCGGTGGCTTCGTC | SEQ ID No. 32 |

Table 7a details alternative redesigned inhA forward primers inhA FW 2, 8, 9 and 11, which may be used successfully in a single multiplex assay in place of SEQ ID No. 23.

TABLE 7a

| | | |
|---|---|---|
| InhA redesigned: inhA | ACGGCAAACGGATTCTGGTT | SEQ ID No. 35 |
| FW2 | | |
| InhA redesigned inhA | TTCTGGTTAGCGGAATCATCACC | SEQ ID No. 36 |
| FW8 | | |
| InhA redesigned inhA | CTGGTTAGCGGAATCATCACCG | SEQ ID No. 37 |
| FW9 | | |
| InhA redesigned inhA | TTAGCGGAATCATCACCGACT | SEQ ID No. 38 |
| FW11 | | |

### Gene Target Regions

Visualized target regions are shown as either the parent or complement strand depending on gene orientation. Target regions were designed to be 900-1100 bp long as this is a good size for PCR and nanopore sequencing. Keeping the PCR products a uniform size reduces bias toward certain targets in multiplex PCR and sequencing reactions.

### Eis

The target region for identified eis mutations encompasses the promoter region, denoted in bold text, of the 1,209 base pair eis gene. The eis gene is on the complement strand. Sequence outside the annotated gene is highlighted in grey. Forward and reverse primer locations are written in italics.

Forward Primer:
[Sequence ID No. 1]
5′-TGTCGGGTACCTTTCGAGC-3′
Reverse Primer:
[Sequence ID No. 2]
5′-TCCATGTACAGCGCCATCC-3′
TGTCGGGTACCTTTCGAGCCGCCGAGCTGACCGCGGCGGAACTAGGTCCCG
CCGTTAGGGTGATCGACTCGAGGTCGGCCGCGATGGGCGTCGGTTTCGCG
GCACTGGCGGCCGGGCGGGCAGCCGCCGCAGGCGATGAGCTGGATACGGT
CGCGCGCGCAGCGGCTGCGGCGGTAAGCCGGATTCACGCGTTCGTCGCTGT
AGCGCGGTTGGACAATCTGCGCCGCAGCGGGCGCATCAGTGGGGCCAAGG
CATGGTTGGGCACCGCGCTGGCGCTCAAGCCGCTGCTGTCAGTCGACGACG
GAAAACTTGTTCTGGTCCAACGGGTTCGCACTGTGAGCAACGCGACGGCGG
TGATGATCGACCGGGTTTGCCAGCTTGTCGGCGACCGCCCCGCCGCTCTCG
CGGTGCATCACGTCGCCGACCCGGCAGCTGCGAACGACGTGGCGGCGGCG
CTGGCGGAGCGGCTGCCGGCGTGTGAGCCGGCCATGGTGACCGCCATGGG
ACCGGTACTTGCTCTGCACGTCGGTGCCGGAGCCGTCGGGGTATGCGTCGA
CGTGGGAGCGTCGCCGCCAGCGTAACGTCACGGCGAAATTCGTCGCTGATT
CTCGCAGTGGCGTCACGCTGGCGGGGCTACCCGCATCGCGTGATCCTTTGC
CAGACACTGTCGTCGTAATATTCACGTGCACGTGGCCGCGGCATATGCCAC
AGTCGGATTCTGGTGACTGTGACCCTGTGTAGCCCGACCGAGGACGACTG
GCCGGGGATGTTCCTACTGGCCGCGGCCAGTTTCACCGATTTCATCGGCCCT
GAATCAGCGACCGCCTGGCGGACCCTGGTGCCCACCGACGGAGCGGTGGT
GGTCCGCGATGGTGCCGGCCCGGGTTCTGAGGTGGTCGGGATGGCGCTGT
ACATGGA

embB

The embB target region on the parent strand is a subsection of the overall 3,297 base pair embB gene. The region chosen contains all the high confidence SNPS and the majority of known embB SNPs. Forward and reverse primer locations are written in italics.

Forward Primer:
[Sequence ID No. 3]
5′-CGCCGTGGTGATATTCGGC-3′
Reverse Primer:
[Sequence ID No. 4]
5′-GCACACCGTAGCTGGAGAC-3′
CGCCGTGGTGATATTCGGCTTCCTGCTCTGGCATGTCATCGGCGCGAATTCG
TCGGACGACGGCTACATCCTGGGCATGGCCCGAGTCGCCGACCACGCCGGC
TACATGTCCAACTATTTCCGCTGGTTCGGCAGCCCGGAGGATCCCTTCGGCT
GGTATTACAACCTGCTGGCGCTGATGACCCATGTCAGCGACGCCAGTCTGT
GGATGCGCCTGCCAGACCTGGCCGCCGGGCTAGTGTGCTGGCTGCTGCTGT
CGCGTGAGGTGCTGCCCCGCCTCGGGCCGGCGGTGGAGGCCAGCAAACCC
GCCTACTGGGCGGCGGCCATGGTCTTGCTGACCGCGTGGATGCCGTTCAAC
AACGGCCTGCGGCCGGAGGGCATCATCGCGCTCGGCTCGCTGGTCACCTAT
GTGCTGATCGAGCGGTCCATGCGGTACAGCCGGCTCACACCGGCGGCGCTG
GCCGTCGTTACCGCCGCATTCACACTGGGTGTGCAGCCCACCGGCCTGATC
GCGGTGGCCGCGCTGGTGGCCGGCGGCCGCCCGATGCTGCGGATCTTGGT
GCGCCGTCATCGCCTGGTCGGCACGTTGCCGTTGGTGTCGCCGATGCTGGC
CGCCGGCACCGTCATCCTGACCGTGGTGTTCGCCGACCAGACCCTGTCAACG
GTGTTGGAAGCCACCAGGGTTCGCGCCAAAATCGGGCCGAGCCAGGCGTG
GTATACCGAGAACCTGCGTTACTACTACCTCATCCTGCCCACCGTCGACGGT
TCGCTGTCGCGGCGCTTCGGCTTTTTGATCACCGCGCTATGCCTGTTCACCG
CGGTGTTCATCATGTTGCGGCGCAAGCGAATTCCCAGCGTGGCCCGCGGAC
CGGCGTGGCGGCTGATGGGCGTCATCTTCGGCACCATGTTCTTCCTGATGT
TCACGCCCACCAAGTGGGTGCACCACTTCGGGCTGTTCGCCGCCGTAGGGG
CGGCGATGGCCGCGCTGACGACGGTGTTGGTATCCCCATCGGTGCTGCGCT
GGTCGCGCAACCGGATGGCGTTCCTGGCGGCGTTATTCTTCCTGCTGGCGT
TGTGTTGGGCCACCACCAACGGCTGGTGGTATGTCTCCAGCTACGGTGTGC

rrs

The rrs primers target includes a subset of the 1,537 base pair rrs gene on the parent strand and some sequence outside the gene at the 3′ end as some of the target SNPs are at the 3′ end of the gene. Sequence outside the annotated gene is highlighted in grey. Forward and reverse primer locations are written in italics.

Forward Primer:
[Sequence ID No. 5]
5′-CTCTGGGCAGTAACTGACGC-3′
Reverse Primer:
[Sequence ID No. 6]
5′-GAGTGTTGCCTCAGGACCC-3′
CTCTGGGCAGTAACTGACGCTGAGGAGCGAAAGCGTGGGGAGCGAACAGG
ATTAGATACCCTGGTAGTCCACGCCGTAAACGGTGGGTACTAGGTGTGGGT
TTCCTTCCTTGGGATCCGTGCCGTAGCTAACGCATTAAGTACCCCGCCTGGG
GAGTACGGCCGCAAGGCTAAAACTCAAAGGAATTGACGGGGGCCCGCACA
AGCGGCGGAGCATGTGGATTAATTCGATGCAACGCGAAGAACCTTACCTGG
GTTTGACATGCACAGGACGCGTCTAGAGATAGGCGTTCCCTTGTGGCCTGT
GTGCAGGTGGTGCATGGCTGTCGTCAGCTCGTGTCGTGAGATGTTGGGTT
AAGTCCCGCAACGAGCGCAACCCTTGTCTCATGTTGCCAGCACGTAATGGTG
GGGACTCGTGAGAGACTGCCGGGGTCAACTCGGAGGAAGGTGGGGATGA
CGTCAAGTCATCATGCCCCTTATGTCCAGGGCTTCACACATGCTACAATGGC
CGGTACAAAGGGCTGCGATGCCGCGAGGTTAAGCGAATCCTTAAAAGCCGG
TCTCAGTTCGGATCGGGGTCTGCAACTCGACCCCGTGAAGTCGGAGTCGCT
AGTAATCGCAGATCAGCAACGCTGCGGTGAATACGTTCCCGGGCCTTGTAC
ACACCGCCCGTCACGTCATGAAAGTCGGTAACACCCGAAGCCAGTGGCCTA
ACCCTCGGGAGGGAGCTGTCGAAGGTGGGATCGGCGATTGGGACGAAGTC
GTAACAAGGTAGCCGTACCGGAAGGTGCGGCTGGATCACCTCCTTTCTAAG
GAGCACCACGAAAACGCCCCAACTGGTGGGGCGTAGGCCGTGAGGGGTTC
TTGTCTGTAGTGGGCGAGAGCCGGGTGCATGACAACAAAGTTGGCCACCAA
CACACTGTTGGGTCCTGAGGCAACACTC

rv0678

The rv0678 target region contains the entire 498 base pair rv0678 gene on the parent strand along with intergenic regions on either side. Sequence outside the annotated gene is highlighted in grey. Forward and reverse primer locations are written in italics.

Forward Primer:
[Sequence ID No. 7]
5′-GCTCGTCCTTCACTTCGCC-3′
Reverse Primer:
[Sequence ID No. 8]
5′-ATCAGTCGTCCTCTCCGGT-3′
GCTCGTCCTTCACTTCGCCATCGACGGTGATTCGGCAGGTGATGGAAGTGCC
GTCGCCTTGCGCGAGGATGTTGGGGGCCGCGGACGGCGCCGTGGTCTTCA
AGGTGAGCGACCACGGCAGGGCTGCGCCGTCGATCCGCTGTGGCTTGGCG
TCGAGGTCCAGGTAGTTGATGTTGACGTAACTACCGGAGCCGGAAACTTCG
TACTCCACCACCTTGGGGTCGAACGGCTCCGGGTCATCGGCGAAGACCTTC
GGCGTCACCAAGATGCCTTCGGAACCAAAGAAAGTGCGGATCCGCTGCACC
GTGAAGCCGGCGATGGCGACCACAACCAGGATGAGCAGCGGTATCCAGGC
ACGCTTGAGAGTTCCAATCATCGCCCTCCGCCTCTGCCGCATGAAGTTCACG
CCGGTCTGGTGACGCATACCGAACGTCACAGATTTCAGAGTACAGTGAAAC
TTGTGAGCGTCAACGACGGGGTCGATCAGATGGGCGCCGAGCCCGACATCA
TGGAATTCGTCGAACAGATGGGCGGCTATTTCGAGTCCAGGAGTTTGACTC
GGTTGGCGGGTCGATTGTTGGGCTGGCTGCTGGTGTGTGATCCCGAGCGG
CAGTCCTCGGAGGAACTGGCGACGGCGCTGGCGGCCAGCAGCGGGGGGAT
CAGCACCAATGCCCGGATGCTGATCCAATTTGGGTTCATTGAGCGGCTCGC
GGTCGCCGGGGATCGGCGCACCTATTTCCGGTTGCGGCCCAACGCTTTCGC
GGCTGGCGAGCGTGAACGCATCCGGGCAATGGCCGAACTGCAGGACCTGG
CTGACGTGGGGCTGAGGGCGCTGGGCGACGCCCCGCCGCAGCGAAGCCGA
CGGCTGCGGGAGATGCGGGATCTGTTGGCATATATGGAGAACGTCGTCTCC
GACGCCCTGGGGCGATACAGCCAGCGAACCGGAGAGGACGACTGAT

fabG1

The fabG1 target region covers the 744 bp fabG1 gene on the parent strand along the gene promoter region (denoted in bold), targeting the high confidence SNPs located therein, and some intergenic sequence at the 3′ end. Sequence outside the annotated gene is highlighted in grey. Forward and reverse primer locations are written in italics.

Forward Primer:
[Sequence ID No. 9]
5′-CTTTTGCACGCAATTGCGC-3′
Reverse Primer:
[Sequence ID No. 10]
5′-AGCAGTCCTGTCATGTGCG-3′
CTTTTGCACGCAATTGCGCGGTCAGTTCCACACCCTGCGGCACGTACACGTC
TTTATGTAGCGCGACATACCTGCTGCGCAATTCGTAGGGCGTCAATACACCC
GCAGCCAGGGCCTCGCTGCCCAGAAAGGGATCCGTCATGGTCGAAGTGTGC
TGAGTCACACCGACAAACGTCACGAGCGTAACCCCAGTGCGAAAGTTCCCG
CCGGAAATCGCAGCCACGTTACGCTCGTGGACATACCGATTTCGGCCCGGC
CGCGGCGAGACGATAGGTTGTCGGGGTGACTGCCACAGCCACTGAAGGGG
CCAAACCCCCATTCGTATCCCGTTCAGTCCTGGTTACCGGAGGAAACCGGGG
GATCGGGCTGGCGATCGCACAGCGGCTGGCTGCCGACGGCCACAAGGTGG
CCGTCACCCACCGTGGATCCGGAGCGCCAAAGGGGCTGTTTGGCGTCGAAT
GTGACGTCACCGACAGCGACGCCGTCGATCGCGCCTTCACGGCGGTAGAAG
AGCACCAGGGTCCGGTCGAGGTGCTGGTGTCCAACGCCGGCCTATCCGCGG
ACGCATTCCTCATGCGGATGACCGAGGAAAAGTTCGAGAAGGTCATCAACG
CCAACCTCACCGGGGCGTTCCGGGTGGCTCAACGGGCATCGCGCAGCATGC
AGCGCAACAAATTCGGTCGAATGATATTCATAGGTTCGGTCTCCGGCAGCT
GGGGCATCGGCAACCAGGCCAACTACGCAGCCTCCAAGGCCGGAGTGATTG
GCATGGCCCGCTCGATCGCCCGCGAGCTGTCGAAGGCAAACGTGACCGCGA
ATGTGGTGGCCCCGGGCTACATCGACACCGATATGACCCGCGCGCTGGATG
AGCGGATTCAGCAGGGGGCGCTGCAATTTATCCCAGCGAAGCGGGTCGGC
ACCCCCGCCGAGGTCGCCGGGGTGGTCAGCTTCCTGGCTTCCGAGGATGCG
AGCTATATCTCCGGTGCGGTCATCCCGGTCGACGGCGGCATGGGTATGGGC
CACTGACACAACACAAGGACGCACATGACAGGACTGCT

gyrA

The gyrA target region is a subset of the overall 2,517 bp gyrA gene on the parent strand. This target region was designed to encompass all the high confidence gyrA resistance-conferring SNPs. Forward and reverse primer locations are written in italics.

Forward Primer:
[Sequence ID No. 11]
5′-TGACAGACACGACGTTGCC-3′
Reverse Primer:
[Sequence ID No. 12]
5′-CGATCGCTAGCATGTTGGC-3′
TGACAGACACGACGTTGCCGCCTGACGACTCGCTCGACCGGATCGAACCGG
TTGACATCGAGCAGGAGATGCAGCGCAGCTACATCGACTATGCGATGAGCG
TGATCGTCGGCCGCGCGCTGCCGGAGGTGCGCGACGGGCTCAAGCCCGTG
CATCGCCGGGTGCTCTATGCAATGTTCGATTCCGGCTTCCGCCCGGACCGCA
GCCACGCCAAGTCGGCCCGGTCGGTTGCCGAGACCATGGGCAACTACCACC
CGCACGGCGACGCGTCGATCTACGACAGCCTGGTGCGCATGGCCCAGCCCT
GGTCGCTGCGCTACCCGCTGGTGGACGGCCAGGGCAACTTCGGCTCGCCAG
GCAATGACCCACCGGCGGCGATGAGGTACACCGAAGCCCGGCTGACCCCGT
TGGCGATGGAGATGCTGAGGGAAATCGACGAGGAGACAGTCGATTTCATC
CCTAACTACGACGGCCGGGTGCAAGAGCCGACGGTGCTACCCAGCCGGTTC
CCCAACCTGCTGGCCAACGGGTCAGGCGGCATCGCGGTCGGCATGGCAACC
AATATCCCGCCGCACAACCTGCGTGAGCTGGCCGACGCGGTGTTCTGGGCG
CTGGAGAATCACGACGCCGACGAAGAGGAGACCCTGGCCGCGGTCATGGG
GCGGGTTAAAGGCCCGGACTTCCCGACCGCCGGACTGATCGTCGGATCCCA
GGGCACCGCTGATGCCTACAAAACTGGCCGCGGCTCCATTCGAATGCGCGG
AGTTGTTGAGGTAGAAGAGGATTCCCGCGGTCGTACCTCGCTGGTGATCAC
CGAGTTGCCGTATCAGGTCAACCACGACAACTTCATCACTTCGATCGCCGAA
CAGGTCCGAGACGGCAAGCTGGCCGGCATTTCCAACATTGAGGACCAGTCT
AGCGATCGGGTCGGTTTACGCATCGTCATCGAGATCAAGCGCGATGCGGTG
GCCAAGGTGGTGATCAATAACCTTTACAAGCACACCCAGCTGCAGACCAGCT
TTGGCGCCAACATGCTAGCGATCG

rpoB

The rpoB target region is a subset of the 3,519 bp rpoB gene on the parent strand. This target region was designed to encompass all the high confidence rpoB resistance-conferring SNPs. Forward and reverse primer locations are written in italics.

Forward Primer:
[Sequence ID No. 13]
5′-TCATCATCAACGGGACCGAG-3′
Reverse Primer:
[Sequence ID No. 14]
5′-ACACGATCTCGTCGCTAACC-3′
TCATCATCAACGGGACCGAGCGTGTGGTGGTCAGCCAGCTGGTGCGGTCGC
CCGGGGTGTACTTCGACGAGACCATTGACAAGTCCACCGACAAGACGCTGC
ACAGCGTCAAGGTGATCCCGAGCCGCGGCGCGTGGCTCGAGTTTGACGTCG
ACAAGCGCGACACCGTCGGCGTGCGCATCGACCGCAAACGCCGGCAACCGG
TCACCGTGCTGCTCAAGGCGCTGGGCTGGACCAGCGAGCAGATTGTCGAGC
GGTTCGGGTTCTCCGAGATCATGCGATCGACGCTGGAGAAGGACAACACCG
TCGGCACCGACGAGGCGCTGTTGGACATCTACCGCAAGCTGCGTCCGGGCG
AGCCCCCGACCAAAGAGTCAGCGCAGACGCTGTTGGAAAACTTGTTCTTCAA
GGAGAAGCGCTACGACCTGGCCCGCGTCGGTCGCTATAAGGTCAACAAGAA
GCTCGGGCTGCATGTCGGCGAGCCCATCACGTCGTCGACGCTGACCGAAGA
AGACGTCGTGGCCACCATCGAATATCTGGTCCGCTTGCACGAGGGTCAGAC
CACGATGACCGTTCCGGGCGGCGTCGAGGTGCCGGTGGAAACCGACGACA
TCGACCACTTCGGCAACCGCCGCCTGCGTACGGTCGGCGAGCTGATCCAAA
ACCAGATCCGGGTCGGCATGTCGCGGATGGAGCGGGTGGTCCGGGAGCG
GATGACCACCCAGGACGTGGAGGCGATCACACCGCAGACGTTGATCAACAT
CCGGCCGGTGGTCGCCGCGATCAAGGAGTTCTTCGGCACCAGCCAGCTGAG
CCAATTCATGGACCAGAACAACCCGCTGTCGGGGTTGACCCACAAGCGCCG
ACTGTCGGCGCTGGGGCCCGGCGGTCTGTCACGTGAGCGTGCCGGGCTGG
AGGTCCGCGACGTGCACCCGTCGCACTACGGCCGGATGTGCCCGATCGAAA
CCCCTGAGGGGCCCAACATCGGTCTGATCGGCTCGCTGTCGGTGTACGCGC
GGGTCAACCCGTTCGGGTTCATCGAAACGCCGTACCGCAAGGTGGTCGACG
GCGTGGTTAGCGACGAGATCGTGT

ethA

The ethA target region covers a subset of the 1470 base pair ethA gene on the complement strand. This section was chosen to cover the high confidence SNPs located at the 5′ end of the gene. Sequence outside the annotated gene is underlined. Forward and reverse primer locations are written italics.

Forward Primer:
[Sequence ID No. 15]
5′-TGGATCCATGACCGAGCAC-3′
Reverse Primer:
[Sequence ID No. 16]
5′-GTCCAGGAGGCATTGGTGT-3′
TGGATCCATGACCGAGCACCTCGACGTTGTCATCGTGGGCGCTGGAATCTC
CGGTGTCAGCGCGGCCTGGCACCTGCAGGACCGTTGCCCGACCAAGAGCTA
CGCCATCCTGGAAAAGCGGGAATCCATGGGCGGCACCTGGGATTTGTTCCG
TTATCCCGGAATTCGCTCCGACTCCGACATGTACACGCTAGGTTTCCGATTC
CGTCCCTGGACCGGACGGCAGGCGATCGCCGACGGCAAGCCCATCCTCGAG
TACGTCAAGAGCACCGCGGCCATGTATGGAATCGACAGGCATATCCGGTTC
CACCACAAGGTGATCAGTGCCGATTGGTCGACCGCGGAAAACCGCTGGACC
GTTCACATCCAAAGCCACGGCACGCTCAGCGCCCTCACCTGCGAATTCCTCT
TTCTGTGCAGCGGCTACTACAACTACGACGAGGGCTACTCGCCGAGATTCG
CCGGCTCGGAGGATTTCGTCGGGCCGATCATCCATCCGCAGCACTGGCCCG
AGGACCTCGACTACGACGCTAAGAACATCGTCGTGATCGGCAGTGGCGCAA
CGGCGGTCACGCTCGTGCCGGCGCTGGCGGACTCGGGCGCCAAGCACGTC
ACGATGCTGCAGCGCTCACCCACCTACATCGTGTCGCAGCCAGACCGGGAC
GGCATCGCCGAGAAGCTCAACCGCTGGCTGCCGGAGACCATGGCCTACACC
GCGGTACGGTGGAAGAACGTGCTGCGCCAGGCGGCCGTGTACAGCGCCTG
CCAGAAGTGGCCACGGCGCATGCGGAAGATGTTCCTGAGCCTGATCCAGCG
CCAGCTACCCGAGGGGTACGACGTGCGAAAGCACTTCGGCCCGCACTACAA
CCCCTGGGACCAGCGATTGTGCTTGGTGCCCAACGGCGACCTGTTCCGGGC
CATTCGTCACGGGAAGGTCGAGGTGGTGACCGACACCATTGAACGGTTCAC
CGCGACCGGAATCCGGCTGAACTCAGGTCGCGAACTGCCGGCTGACATCAT
CATTACCGCAACGGGGTTGAACCTGCAGCTTTTTGGTGGGGCGACGGCGAC
TATCGACGGACAACAAGTGGACATCACCACGACGATGGCCTACAAGGGCAT
GATGCTTTCCGGCATCCCCAACATGGCCTACACGGTTGGCTACACCAATGCC
TCCTGGAC

rplC

The rplC target region contains the entire 654 bp rplC gene on the parent strand along with intergenic regions on the 5′ and 3′ ends. Sequence outside the annotated gene is highlighted in grey. Forward and reverse primer locations are written in italics.

Forward Primer:
[Sequence ID No. 17]
5′-AGTACAAGGACTCGCGGGA-3′
Reverse Primer:
[Sequence ID No. 18]
5′-TCGAGTGGGTACCCTGGC-3′
AGTACAAGGACTCGCGGGAGCACTTCGAGATGCGCACACACAAGCGGTTGA
TCGACATCATCGATCCCACGCCGAAGACCGTTGACGCGCTCATGCGCATCGA
CCTTCCGGCCAGCGTCGACGTCAACATCCAGTAGGAGATTGGACAGAGCAA
TGGCACGAAAGGGCATTCTCGGTACCAAGCTGGGTATGACGCAGGTATTCG
ACGAAAGCAACAGAGTAGTACCGGTGACCGTGGTCAAGGCCGGGCCCAAC
GTGGTAACCCGCATCCGCACGCCCGAACGCGACGGTTATAGCGCCGTGCAG
CTGGCCTATGGCGAGATCAGCCCACGCAAGGTCAACAAGCCGCTGACAGGT
CAGTACACCGCCGCCGGCGTCAACCCACGCCGATACCTGGCGGAGCTGCGG
CTGGACGACTCGGATGCCGCGACCGAGTACCAGGTTGGGCAAGAGTTGACC
GCGGAGATCTTCGCCGATGGCAGCTACGTCGATGTGACGGGTACCTCCAAG
GGCAAAGGTTTCGCCGGCACCATGAAGCGGCACGGCTTCCGCGGTCAGGG
CGCCAGTCACGGTGCCCAGGCGGTGCACCGCCGTCCGGGCTCCATCGGCGG
ATGTGCCACGCCGGCGCGGGTGTTCAAGGGCACCCGGATGGCCGGGCGGA
TGGGCAATGACCGGGTGACCGTTCTTAACCTTTTGGTGCATAAGGTCGATG
CCGAGAACGGCGTGCTGCTGATCAAGGGTGCGGTTCCTGGCCGCACCGGT
GGACTGGTCATGGTCCGCAGTGCGATCAAACGAGGTGAGAAGTGATGGCT
GCGCAAGAGCAGAAGACACTCAAAATCGACGTCAAGACGCCGGCGGGCAA
GGTCGACGGCGCTATCGAGCTGCCGGCCGAGCTGTTCGACGTCCCGGCCAA
CATCGCGCTGATGCACCAGGTGGTCACCGCCCAGCGGGCGGCGGCACGCCA
GGGTACCCACTCGA

katG (Initial Primer Pair)

The katG target region is a subset of the 2,223 base pair katG gene, which is on the complement strand. The region was chosen to cover all high confidence SNPs. Forward and reverse primer locations are highlighted in italics.

Forward Primer:
[Sequence ID No. 19]
5′-CTGTGGCCGGTCAAGAAGA-3′
Reverse Primer:
[Sequence ID No. 33]
5′-TGCCCGGATCTGGCTCTTA-3′
CTGTGGCCGGTCAAGAAGAAGTACGGCAAGAAGCTCTCATGGGCGGACCTGATTG
TTTTCGCCGGCAACTGCGCGCTGGAATCGATGGGCTTCAAGACGTTCGGGTTCGG
CTTCGGCCGGGTCGACCAGTGGGAGCCCGATGAGGTCTATTGGGGCAAGGAAGC
CACCTGGCTCGGCGATGAGCGTTACAGCGGTAAGCGGGATCTGGAGAACCCGCTG
GCCGCGGTGCAGATGGGGCTGATCTACGTGAACCCGGAGGGGCCGAACGGCAAC
CCGGACCCCATGGCCGCGGCGGTCGACATTCGCGAGACGTTTCGGCGCATGGCCA
TGAACGACGTCGAAACAGCGGCGCTGATCGTCGGCGGTCACACTTTCGGTAAGAC
CCATGGCGCCGGCCCGGCCGATCTGGTCGGCCCCGAACCCGAGGCTGCTCCGCTG
GAGCAGATGGGCTTGGGCTGGAAGAGCTCGTATGGCACCGGAACCGGTAAGGAC
GCGATCACCAGCGGCATCGAGGTCGTATGGACGAACACCCCGACGAAATGGGACA
ACAGTTTCCTCGAGATCCTGTACGGCTACGAGTGGGAGCTGACGAAGAGCCCTGC
TGGCGCTTGGCAATACACCGCCAAGGACGGCGCCGGTGCCGGCACCATCCCGGAC
CCGTTCGGCGGGCCAGGGCGCTCCCCGACGATGCTGGCCACTGACCTCTCGCTGC
GGGTGGATCCGATCTATGAGCGGATCACGCGTCGCTGGCTGGAACACCCCGAGGA
ATTGGCCGACGAGTTCGCCAAGGCCTGGTACAAGCTGATCCACCGAGACATGGGT
CCCGTTGCGAGATACCTTGGGCCGCTGGTCCCCAAGCAGACCCTGCTGTGGCAGG
ATCCGGTCCCTGCGGTCAGCCACGACCTCGTCGGCGAAGCCGAGATTGCCAGCCTT
AAGAGCCAGATCCGGGCA

katG—Redesigned

The katG target region is a subset of the 2,223 bp katG gene, which is on the complement strand. The region was chosen to cover all the high confidence SNPs. Forward and reverse primer locations are written in italics.

Forward Primer:
[Sequence ID No. 19]
5′-CTGTGGCCGGTCAAGAAGA-3′
Reverse Primer:
[Sequence ID No. 20]
5′-GGATCTGGCTCTTAAGGCTGG-3′
CTGTGGCCGGTCAAGAAGAAGTACGGCAAGAAGCTCTCATGGGCGGACCTG
ATTGTTTTCGCCGGCAACTGCGCGCTGGAATCGATGGGCTTCAAGACGTTC
GGGTTCGGCTTCGGCCGGGTCGACCAGTGGGAGCCCGATGAGGTCTATTG
GGGCAAGGAAGCCACCTGGCTCGGCGATGAGCGTTACAGCGGTAAGCGGG
ATCTGGAGAACCCGCTGGCCGCGGTGCAGATGGGGCTGATCTACGTGAACC
CGGAGGGGCCGAACGGCAACCCGGACCCCATGGCCGCGGCGGTCGACATT
CGCGAGACGTTTCGGCGCATGGCCATGAACGACGTCGAAACAGCGGCGCT
GATCGTCGGCGGTCACACTTTCGGTAAGACCCATGGCGCCGGCCCGGCCGA
TCTGGTCGGCCCCGAACCCGAGGCTGCTCCGCTGGAGCAGATGGGCTTGG
GCTGGAAGAGCTCGTATGGCACCGGAACCGGTAAGGACGCGATCACCAGC
GGCATCGAGGTCGTATGGACGAACACCCCGACGAAATGGGACAACAGTTTC
CTCGAGATCCTGTACGGCTACGAGTGGGAGCTGACGAAGAGCCCTGCTGGC
GCTTGGCAATACACCGCCAAGGACGGCGCCGGTGCCGGCACCATCCCGGAC
CCGTTCGGCGGGCCAGGGCGCTCCCCGACGATGCTGGCCACTGACCTCTCG
CTGCGGGTGGATCCGATCTATGAGCGGATCACGCGTCGCTGGCTGGAACAC
CCCGAGGAATTGGCCGACGAGTTCGCCAAGGCCTGGTACAAGCTGATCCAC
CGAGACATGGGTCCCGTTGCGAGATACCTTGGGCCGCTGGTCCCCAAGCAG
ACCCTGCTGTGGCAGGATCCGGTCCCTGCGGTCAGCCACGACCTCGTCGGC
GAAGCCGAGATTGCCAGCCTTAAGAGCCAGATCCGGGCA

gidB

The gidB target region contains the entire 675 bp gidB gene on the parent strand along with intergenic sequence on the 5′ and 3′ ends. Sequence outside the annotated gene is highlighted in grey. Forward and reverse primer locations are written in italics.

Forward Primer:
[Sequence ID No. 21]
5′-TGACACAGACCTCACGAGC-3′
Reverse Primer:
[Sequence ID No. 22]
5′-GCCCTTCTGATTCGCGATG-3′
TGACACAGACCTCAGGAGCCGGCGGAGTGCGTAATGTCTCCGATCGAGCCC
GCGGCGTCTGCGATCTTCGGACCGCGGCTTGGCCTTGCTCGGCGGTACGCC
GAAGCGTTGGCGGGACCCGGTGTGGAGCGGGGGCTGGTGGGACCCCGCG
AAGTCGGTAGGCTATGGGACCGGCATCTACTGAACTGCGCCGTGATCGGTG
AGCTCCTCGAACGCGGTGACCGGGTCGTGGATATCGGTAGCGGAGCCGGG
TTGCCGGGCGTGCCATTGGCGATAGCGCGGCCGGACCTCCAGGTAGTTCTC
CTAGAACCGCTACTGCGCCGCACCGAGTTTCTTCGAGAGATGGTGACAGAT
CTGGGCGTGGCCGTTGAGATCGTGCGGGGGCGCGCCGAGGAGTCCTGGGT
GCAGGACCAATTGGGCGGCAGCGACGCTGCGGTGTCACGGGCGGTGGCCG
CGTTGGACAAGTTGACGAAATGGAGCATGCCGTTGATACGGCCGAACGGG
CGAATGCTCGCCATCAAAGGCGAGCGGGCTCACGACGAAGTACGGGAGCA
CCGGCGTGTGATGATCGCATCGGGCGCGGTTGATGTCAGGGTGGTGACAT
GTGGCGCGAACTATTTGCGTCCGCCCGCGACCGTGGTGTTCGCACGACGTG
GAAAGCAGATCGCCCGAGGGTCGGCACGGATGGCGAGTGGAGGGACGGC
GTGAGTGCTCCGTGGGGCCCGGTGGCCGCTGGACCGTCCGCGCTCGTAAG
GTCGGGCCAGGCTTCAACTATCGAACCATTCCAGCGGGAAATGACACCACC
GACACCGACGCCTGAGGCCGCGCACAATCCGACGATGAATGTTTCACGTGA
AACATCGACAGAATTCGACACCCCCATCGGCGCTGCAGCAGAACGTGCGAT
GCGGGTCCTGCACACCACCCACGAGCCGCTGCAGCGGCCGGGTCGACGCCG
GGTGCTCACCATCGCGAATCAGAAGGGC

inhA—Initial Primer Pair

The inhA target region contains a subset of the inhA 810 bp gene on the parent strand along with the promoter region, denoted in bold, to cover all the high confidence SNPs in the gene and promotor. Sequence outside the annotated gene is highlighted in grey. Forward and reverse primer locations are highlighted in italics.

Forward Primer:
[Sequence ID No. 34]
5′-GGGCGCTGCAATTTATCCC-3′
Reverse Primer:
[Sequence ID No. 24]
5′-GGCGTAGATGATGTCACCC-3′
GGGCGCTGCAATTTATCCCAGCGAAGCGGGTCGGCACCCCCGCCGAGGTCG
CCGGGGTGGTCAGCTTCCTGGCTTCCGAGGATGCGAGCTATATCTCCGGTG
CGGTCATCCCGGTCGACGGCGGCATGGGTATGGGCCACTGACACAACACAA
GGACGCACATGACAGGACTGCTGGACGGCAAACGGATTCTGGTTAGCGGA
ATCATCACCGACTCGTCGATCGCGTTTCACATCGCACGGGTAGCCCAGGAGC
AGGGCGCCCAGCTGGTGCTCACCGGGTTCGACCGGCTGCGGCTGATTCAGC
GCATCACCGACCGGCTGCCGGCAAAGGCCCCGCTGCTCGAACTCGACGTGC
AAAACGAGGAGCACCTGGCCAGCTTGGCCGGCCGGGTGACCGAGGCGATC
GGGGGGGGCAACAAGCTCGACGGGGTGGTGCATTCGATTGGGTTCATGCC
GCAGACCGGGATGGGCATCAACCCGTTCTTCGACGCGCCCTACGCGGATGT
GTCCAAGGGCATCCACATCTCGGCGTATTCGTATGCTTCGATGGCCAAGGC
GCTGCTGCCGATCATGAACCCCGGAGGTTCCATCGTCGGCATGGACTTCGA
CCCGAGCCGGGCGATGCCGGCCTACAACTGGATGACGGTCGCCAAGAGCG
CGTTGGAGTCGGTCAACAGGTTCGTGGCGCGCGAGGCCGGCAAGTACGGT
GTGCGTTCGAATCTCGTTGCCGCAGGCCCTATCCGGACGCTGGCGATGAGT
GCGATCGTCGGCGGTGCGCTCGGCGAGGAGGCCGGCGCCCAGATCCAGCT
GCTCGAGGAGGGCTGGGATCAGCGCGCTCCGATCGGCTGGAACATGAAGG
ATGCGACGCCGGTCGCCAAGACGGTGTGCGCGCTGCTGTCTGACTGGCTGC
CGGCGACCACGGGTGACATCATCTACGCC

Redesigned inhA Primers

In the following, inhA start and end gene codons are denoted in bold. Forward and reverse primer locations are highlighted in italics.

inhA FW 6

Forward Primer:
[Sequence ID No. 23]
5′-CGGATTCTGGTTAGCGGAATCA-3′
Reverse Primer:
[Sequence ID No. 24]
5′-GGCGTAGATGATGTCACCC-3′
ATGACAGGACTGCTGGACGGCAAACGGATTCTGGTTAGCGGAATCATCACCGACTC
GTCGATCGCGTTTCACATCGCACGGGTAGCCCAGGAGCAGGGCGCCCAGCTGGTG
CTCACCGGGTTCGACCGGCTGCGGCTGATTCAGCGCATCACCGACCGGCTGCCGG
CAAAGGCCCCGCTGCTCGAACTCGACGTGCAAAACGAGGAGCACCTGGCCAGCTT
GGCCGGCCGGGTGACCGAGGCGATCGGGGGGGGCAACAAGCTCGACGGGGTGG
TGCATTCGATTGGGTTCATGCCGCAGACCGGGATGGGCATCAACCCGTTCTTCGAC
GCGCCCTACGCGGATGTGTCCAAGGGCATCCACATCTCGGCGTATTCGTATGCTTC
GATGGCCAAGGCGCTGCTGCCGATCATGAACCCCGGAGGTTCCATCGTCGGCATG
GACTTCGACCCGAGCCGGGCGATGCCGGCCTACAACTGGATGACGGTCGCCAAGA
GCGCGTTGGAGTCGGTCAACAGGTTCGTGGCGCGCGAGGCCGGCAAGTACGGTG
TGCGTTCGAATCTCGTTGCCGCAGGCCCTATCCGGACGCTGGCGATGAGTGCGAT
CGTCGGCGGTGCGCTCGGCGAGGAGGCCGGCGCCCAGATCCAGCTGCTCGAGGA
GGGCTGGGATCAGCGCGCTCCGATCGGCTGGAACATGAAGGATGCGACGCCGGT
CGCCAAGACGGTGTGCGCGCTGCTGTCTGACTGGCTGCCGGCGACCACGGGTGAC
ATCATCTACGCCGACGGCGGCGCGCACACCCAATTGCTCTAG

inhA FW 2

Forward Primer:
[Sequence ID No. 35]
5′-ACGGCAAACGGATTCTGGTT-3′
Reverse Primer:
[Sequence ID No. 24]
5′-GGCGTAGATGATGTCACCC-3′
ATGACAGGACTGCTGGACGGCAAACGGATTCTGGTTAGCGGAATCATCACCGACTC
GTCGATCGCGTTTCACATCGCACGGGTAGCCCAGGAGCAGGGCGCCCAGCTGGTG
CTCACCGGGTTCGACCGGCTGCGGCTGATTCAGCGCATCACCGACCGGCTGCCGG
CAAAGGCCCCGCTGCTCGAACTCGACGTGCAAAACGAGGAGCACCTGGCCAGCTT
GGCCGGCCGGGTGACCGAGGCGATCGGGGCGGGCAACAAGCTCGACGGGGTGG
TGCATTCGATTGGGTTCATGCCGCAGACCGGGATGGGCATCAACCCGTTCTTCGAC
GCGCCCTACGCGGATGTGTCCAAGGGCATCCACATCTCGGCGTATTCGTATGCTTC
GATGGCCAAGGCGCTGCTGCCGATCATGAACCCCGGAGGTTCCATCGTCGGCATG
GACTTCGACCCGAGCCGGGCGATGCCGGCCTACAACTGGATGACGGTCGCCAAGA
GCGCGTTGGAGTCGGTCAACAGGTTCGTGGCGCGCGAGGCCGGCAAGTACGGTG
TGCGTTCGAATCTCGTTGCCGCAGGCCCTATCCGGACGCTGGCGATGAGTGCGAT
CGTCGGCGGTGCGCTCGGCGAGGAGGCCGGCGCCCAGATCCAGCTGCTCGAGGA
GGGCTGGGATCAGCGCGCTCCGATCGGCTGGAACATGAAGGATGCGACGCCGGT
CGCCAAGACGGTGTGCGCGCTGCTGTCTGACTGGCTGCCGGCGACCACGGGTGAC
ATCATCTACGCCGACGGCGGCGCGCACACCCAATTGCTCTAG

inhA FW 8

Forward Primer:
[Sequence ID No. 36]
5′-TTCTGGTTAGCGGAATCATCACC-3′
Reverse Primer:
[Sequence ID No. 24]
5′-GGCGTAGATGATGTCACCC-3′
ATGACAGGACTGCTGGACGGCAAACGGATTCTGGTTAGCGGAATCATCACCGACTC
GTCGATCGCGTTTCACATCGCACGGGTAGCCCAGGAGCAGGGCGCCCAGCTGGTG
CTCACCGGGTTCGACCGGCTGCGGCTGATTCAGCGCATCACCGACCGGCTGCCGG
CAAAGGCCCCGCTGCTCGAACTCGACGTGCAAAACGAGGAGCACCTGGCCAGCTT
GGCCGGCCGGGTGACCGAGGCGATCGGGGGGGGCAACAAGCTCGACGGGGTGG
TGCATTCGATTGGGTTCATGCCGCAGACCGGGATGGGCATCAACCCGTTCTTCGAC
GCGCCCTACGCGGATGTGTCCAAGGGCATCCACATCTCGGCGTATTCGTATGCTTC
GATGGCCAAGGCGCTGCTGCCGATCATGAACCCCGGAGGTTCCATCGTCGGCATG
GACTTCGACCCGAGCCGGGCGATGCCGGCCTACAACTGGATGACGGTCGCCAAGA
GCGCGTTGGAGTCGGTCAACAGGTTCGTGGCGCGCGAGGCCGGCAAGTACGGTG
TGCGTTCGAATCTCGTTGCCGCAGGCCCTATCCGGACGCTGGCGATGAGTGCGAT
CGTCGGCGGTGCGCTCGGCGAGGAGGCCGGCGCCCAGATCCAGCTGCTCGAGGA
GGGCTGGGATCAGCGCGCTCCGATCGGCTGGAACATGAAGGATGCGACGCCGGT
CGCCAAGACGGTGTGCGCGCTGCTGTCTGACTGGCTGCCGGCGACCACGGGTGAC
ATCATCTACGCCGACGGCGGCGCGCACACCCAATTGCTCTAG

inhA FW 9

Forward Primer:
[Sequence ID No. 37]
5′-CTGGTTAGCGGAATCATCACCG-3′
Reverse Primer:
[Sequence ID No. 24]
5′-GGCGTAGATGATGTCACCC-3′
ATGACAGGACTGCTGGACGGCAAACGGATTCTGGTTAGCGGAATCATCACCGACTC
GTCGATCGCGTTTCACATCGCACGGGTAGCCCAGGAGCAGGGCGCCCAGCTGGTG
CTCACCGGGTTCGACCGGCTGCGGCTGATTCAGCGCATCACCGACCGGCTGCCGG
CAAAGGCCCCGCTGCTCGAACTCGACGTGCAAAACGAGGAGCACCTGGCCAGCTT
GGCCGGCCGGGTGACCGAGGCGATCGGGGGGGGCAACAAGCTCGACGGGGTGG
TGCATTCGATTGGGTTCATGCCGCAGACCGGGATGGGCATCAACCCGTTCTTCGAC
GCGCCCTACGCGGATGTGTCCAAGGGCATCCACATCTCGGCGTATTCGTATGCTTC
GATGGCCAAGGCGCTGCTGCCGATCATGAACCCCGGAGGTTCCATCGTCGGCATG
GACTTCGACCCGAGCCGGGCGATGCCGGCCTACAACTGGATGACGGTCGCCAAGA
GCGCGTTGGAGTCGGTCAACAGGTTCGTGGCGCGCGAGGCCGGCAAGTACGGTG
TGCGTTCGAATCTCGTTGCCGCAGGCCCTATCCGGACGCTGGCGATGAGTGCGAT
CGTCGGCGGTGCGCTCGGCGAGGAGGCCGGCGCCCAGATCCAGCTGCTCGAGGA
GGGCTGGGATCAGCGCGCTCCGATCGGCTGGAACATGAAGGATGCGACGCCGGT
CGCCAAGACGGTGTGCGCGCTGCTGTCTGACTGGCTGCCGGCGACCACGGGTGAC
ATCATCTACGCCGACGGCGGCGCGCACACCCAATTGCTCTAG

inhA FW 11

Forward Primer:
[Sequence ID No. 38]
5′-TTAGCGGAATCATCACCGACT-3′
Reverse Primer:
[Sequence ID No. 24]
5′-GGCGTAGATGATGTCACCC-3′
ATGACAGGACTGCTGGACGGCAAACGGATTCTGGTTAGCGGAATCATCACCGACT
CGTCGATCGCGTTTCACATCGCACGGGTAGCCCAGGAGCAGGGCGCCCAGCTGGT
GCTCACCGGGTTCGACCGGCTGCGGCTGATTCAGCGCATCACCGACCGGCTGCCG
GCAAAGGCCCCGCTGCTCGAACTCGACGTGCAAAACGAGGAGCACCTGGCCAGCT
TGGCCGGCCGGGTGACCGAGGCGATCGGGGGGGGCAACAAGCTCGACGGGGTG
GTGCATTCGATTGGGTTCATGCCGCAGACCGGGATGGGCATCAACCCGTTCTTCGA
CGCGCCCTACGCGGATGTGTCCAAGGGCATCCACATCTCGGCGTATTCGTATGCTT
CGATGGCCAAGGCGCTGCTGCCGATCATGAACCCCGGAGGTTCCATCGTCGGCAT
GGACTTCGACCCGAGCCGGGCGATGCCGGCCTACAACTGGATGACGGTCGCCAAG
AGCGCGTTGGAGTCGGTCAACAGGTTCGTGGCGCGCGAGGCCGGCAAGTACGGT
GTGCGTTCGAATCTCGTTGCCGCAGGCCCTATCCGGACGCTGGCGATGAGTGCGA
TCGTCGGCGGTGCGCTCGGCGAGGAGGCCGGCGCCCAGATCCAGCTGCTCGAGG
AGGGCTGGGATCAGCGCGCTCCGATCGGCTGGAACATGAAGGATGCGACGCCGG
TCGCCAAGACGGTGTGCGCGCTGCTGTCTGACTGGCTGCCGGCGACCACGGGTGA
CATCATCTACGCCGACGGCGGCGCGCACACCCAATTGCTCTAG

rrl

The rrl target region is a subsection of the overall 3,138 bp rrl gene on the parent strand, targeting all the high confidence SNPs. Forward and reverse primer locations are written in italics.

Forward Primer:
[Sequence ID No. 25]
5′-GGTCCGTGCGAAGTCGC-3′
Reverse Primer:
[Sequence ID No. 26]
5′-TGAACCCGTGTTCTGCGG-3′
GGTCCGTGCGAAGTCGCAAGACGATGTATACGGACTGACGCCTGCCCGGTG
CTGGAAGGTTAAGAGGACCCGTTAACCCGCAAGGGTGAAGCGGAGAATTT
AAGCCCCAGTAAACGGCGGTGGTAACTATAACCATCCTAAGGTAGCGAAAT
TCCTTGTCGGGTAAGTTCCGACCTGCACGAATGGCGTAACGACTTCTCAACT
GTCTCAACCATAGACTCGGCGAAATTGCACTACGAGTAAAGATGCTCGTTAC
GCGCGGCAGGACGAAAAGACCCCGGGACCTTCACTACAACTTGGTATTGAT
GTTCGGTACGGTTTGTGTAGGATAGGTGGGAGACTGTGAAACCTCGACGCC
AGTTGGGGCGGAGTCGTTGTTGAAATACCACTCTGATCGTATTGGGCATCT
AACCTCGAACCCTGAATCGGGTTTAGGGACAGTGCCTGGCGGGTAGTTTAA
CTGGGGCGGTTGCCTCCTAAAATGTAACGGAGGCGCCCAAAGGTTCCCTCA
ACCTGGACGGCAATCAGGTGGCGAGTGTAAATGCACAAGGGAGCTTGACT
GCGAGACTTACAAGTCAAGCAGGGACGAAAGTCGGGATTAGTGATCCGGC
ACCCCCGAGTGGAAGGGGTGTCGCTCAACGGATAAAAGGTACCCCGGGGA
TAACAGGCTGATCTTCCCCAAGAGTCCATATCGACGGGATGGTTTGGCACCT
CGATGTCGGCTCGTCGCATCCTGGGGCTGGAGCAGGTCCCAAGGGTTGGG
CTGTTCGCCCATTAAAGCGGCACGCGAGCTGGGTTTAGAACGTCGTGAGAC
AGTTCGGTCTCTATCCGCCGCGCGCGTCAGAAACTTGAGGAAACCTGTCCCT
AGTACGAGAGGACCGGGACGGACGAACCTCTGGTGCACCAGTTGTCCCGCC
AGGGGCACCGCTGGATAGCCACGTTCGGTCAGGATAACCGCTGAAAGCATC
TAAGCGGGAAACCTTCTCCAAGATCAGGTTTCTCACCCACTTGGTGGGATAA
GGCCCCCCGCAGAACACGGGTTCA

pncA

The pncA target region contains the entire 561 base pair pncA gene on the complement strand along with intergenic regions at the 5′ and 3′ ends. Sequence outside the annotated gene is highlighted in grey. Forward and reverse primer locations are written in italics.

Forward Primer:
[Sequence ID No. 27]
5′-TCACCGGACGGATTTGTCG-3′
Reverse Primer:
[Sequence ID No. 28]
5′-TCCAGATCGCGATGGAACG-3′
TCACCGGACGGATTTGTCGCTCACTACATCACCGGCGTGATCTATCCCGCCG
GTTGGGTGGCCGCCGCTCAGCTGGTCATGTTCGCGATCGTCGCGGCGTCAT
GGACCCTATATCTGTGGCTGCCGCGTCGGTAGGCAAACTGCCCGGGCAGTC
GCCCGAACGTATGGTGGACGTATGCGGGCGTTGATCATCGTCGACGTGCAG
AACGACTTCTGCGAGGGTGGCTCGCTGGCGGTAACCGGTGGCGCCGCGCT
GGCCCGCGCCATCAGCGACTACCTGGCCGAAGCGGCGGACTACCATCACGT
CGTGGCAACCAAGGACTTCCACATCGACCCGGGTGACCACTTCTCCGGCACA
CCGGACTATTCCTCGTCGTGGCCACCGCATTGCGTCAGCGGTACTCCCGGCG
CGGACTTCCATCCCAGTCTGGACACGTCGGCAATCGAGGCGGTGTTCTACA
AGGGTGCCTACACCGGAGCGTACAGCGGCTTCGAAGGAGTCGACGAGAAC
GGCACGCCACTGCTGAATTGGCTGCGGCAACGCGGCGTCGATGAGGTCGA
TGTGGTCGGTATTGCCACCGATCATTGTGTGCGCCAGACGGCCGAGGACGC
GGTACGCAATGGCTTGGCCACCAGGGTGCTGGTGGACCTGACAGCGGGTG
TGTCGGCCGATACCACCGTCGCCGCGCTGGAGGAGATGCGCACCGCCAGCG
TCGAGTTGGTTTGCAGCTCCTGATGGCACCGCCGAACCGGGATGAACTGTT
GGCGGCGGTGGAGCGCTCGCCGCAAGCGGCCGCCGCGCACGACCGCGCCG
GCTGGGTCGGGTTGTTCACCGGTGACGCGCGGGTCGAAGACCCGGTGGGT
TCGCAGCCGCAGGTGGGGCATGAGGCCATCGGCCGCTTCTACGACACCTTC
ATCGGGCCGCGGGATATCACGTTCCATCGCGATCTGGA

rpsL

The rpsL target region contains the entire 375 bp rpsL gene on the parent strand along with intergenic regions at the 5′ and 3′ ends. Sequence outside the annotated gene is highlighted in grey. Forward and reverse primer locations are written in italics.

Forward Primer:
[Sequence ID No. 29]
5′-GCGGCGGGTATTGTGGTT-3′
Reverse Primer:
[Sequence ID No. 30]
5′-TAACCGGCGCTTCTCACC-3′
GCGGCGGGTATTGTGGTTGCTCGTGCCTGGCGGCTTACGCTTGATGTAGGG
GCGTGGATGCCGGGCCAATTCGCATGTCCGCGATGCCTCGGATGAGACGAA
TCGAGTTTGAGGCAAGCTATGCGACACACCCGGCCGCGGGTAACCGTGGCG
GGGCATGGCCGACAAACAGAACGTGAAAGCGCCCAAGATAGAAAGCCGGT
AGATGCCAACCATCCAGCAGCTGGTCCGCAAGGGTCGTCGGGACAAGATCA
GTAAGGTCAAGACCGCGGCTCTGAAGGGCAGCCCGCAGCGTCGTGGTGTA
TGCACCCGCGTGTACACCACCACTCCGAAGAAGCCGAACTCGGCGCTTCGG
AAGGTTGCCCGCGTGAAGTTGACGAGTCAGGTCGAGGTCACGGCGTACATT
CCCGGCGAGGGCCACAACCTGCAGGAGCACTCGATGGTGCTGGTGCGCGG
CGGCCGGGTGAAGGACCTGCCTGGTGTGCGCTACAAGATCATCCGCGGTTC
GCTGGATACGCAGGGTGTCAAGAACCGCAAACAGGCACGCAGCCGTTACG
GCGCTAAGAAGGAGAAGGGCTGATGCCACGCAAGGGGCCCGCGCCCAAGC
GTCCGTTGGTCAACGACCCGGTCTACGGATCGCAGTTGGTCACCCAGTTGG
TGAACAAGGTTCTGTTGAAGGGGAAAAAATCGCTGGCCGAGCGCATTGTTT
ATGGTGCGCTTGAGCAAGCTCGCGACAAGACCGGCACCGATCCGGTGATCA
CCCTCAAGCGGGCTCTCGACAATGTCAAACCCGCCCTGGAGGTGCGCAGCC
GTCGCGTCGGCGGCGCGACCTATCAGGTGCCTGTCGAGGTGCGCCCCGACC
GGTCGACCACGCTGGCGCTGCGCTGGCTCGTCGGCTACTCGCGGCAACGCC
GTGAGAAGACGATGATCGAGCGCCTGGCAAATGGAGATCCTGGATGCCAG
CAATGGCCTTGGGGCCTCCGTCAAGCGGCGTGAGGACACCCACAAGATGGC
CGAGGCGAACCGAGCCTTTGCGCATTATCGCTGGTGAGAAGCGCCGGTTA

tlyA

The tlyA target region contains the entire 807 base pair tlyA gene on the parent strand along with intergenic regions at the 5′ and 3′ ends. Sequence outside the annotated gene is highlighted in grey. Forward and reverse primer locations are written in italics.

Forward Primer:
[Sequence ID No. 31]
5′-CGTTGATGCGCAGCGATC-3′
Reverse Primer:
[Sequence ID No. 32]
5′-GGTCTCGGTGGCTTCGTC-3′
CGTTGATGCGCAGCGATCATCCGGTGACTAGCGTAGGAACGCAATGACCATC
GATCCTGACCAGATCCGTGCCGAAATCGACGCCCTACTTGCTTCGCTGCCCG
ACCCCGCCGACGCCGAGAACGGACCGTCTCTGGCCGAACTCGAAGGCATCG
CACGTCGTCTTTCCGAGGCGCACGAGGTGTTGTTGGCCGCCCTGGAGTCGG
CGGAGAAGGGTTGAGTGCGGCGTGGCACGACGTGCCCGCGTTGACGCCGA
GCTAGTCCGGCGGGGCCTGGCGCGATCACGTCAACAGGCCGCGGAGTTGA
TCGGCGCCGGCAAGGTGCGCATCGACGGGCTGCCGGCGGTCAAGCCGGCC
ACCGCCGTGTCCGACACCACCGCGCTGACCGTGGTGACCGACAGTGAACGC
GCCTGGGTATCGCGCGGAGCGCACAAACTAGTCGGTGCGCTGGAGGCGTT
CGCGATCGCGGTGGCGGGCCGGCGCTGTCTGGACGCGGGCGCATCGACCG
GTGGGTTCACCGAAGTACTGCTGGACCGTGGTGCCGCCCACGTGGTGGCC
GCCGATGTCGGATACGGCCAGCTGGCGTGGTCGCTGCGCAACGATCCTCGG
GTGGTGGTCCTCGAGCGGACCAACGCACGTGGCCTCACACCGGAGGCGATC
GGCGGTCGCGTCGACCTGGTAGTGGCCGACCTGTCGTTCATCTCGTTGGCT
ACCGTGTTGCCCGCGCTGGTTGGATGCGCTTCGCGCGACGCCGATATCGTT
CCACTGGTGAAGCCGCAGTTTGAGGTGGGGAAAGGTCAGGTCGGCCCCGG
TGGGGTGGTCCATGACCCGCAGTTGCGTGCGCGGTCGGTGCTCGCGGTCG
CGCGGCGGGCACAGGAGCTGGGCTGGCACAGCGTCGGCGTCAAGGCCAGC
CCGCTGCCGGGCCCATCGGGCAATGTCGAGTACTTCCTGTGGTTGCGCACG
CAGACCGACCGGGCATTGTCGGCCAAGGGATTGGAGGATGCGGTGCACCG
TGCGATTAGCGAGGGCCCGTAGTGACCGCTCATCGCAGTGTTCTGCTGGTC
GTCCACACCGGGCGCGACGAAGCCACCGAGACC

### Advantages

The present disclosure provides a means of accurately and rapidly identifying the presence of multiple drug resistance mutations in a sample from a patient with suspected or confirmed Tuberculosis in one or more multiplex reactions, and in preferred embodiments, in a single multiplex reaction. Such information informs decisions regarding drug administration, and allows a tailored regimen to be determined for the patient depending upon the identified mutations. Furthermore, the disclosed methods can be successfully carried out on samples taken directly from patients, such as sputum, thereby adding to their potential for use in lower and middle income and developing countries. The development of optimised primers for this purpose means the advantages of using a multiplex assay can be realised. The disclosed methods are highly sensitive (<100 MTB cells), rapid (taking approximately 8 hours) and can detect a broad range of mutations, and thus represent a major improvement over current culture, molecular (e.g. GenoType MTBDRsl line probe assay) and tNGS based tests. This allows the correct treatment pathway to be determined and for patients to commence treatment promptly and not be lost to follow-up (a major problem in developing countries). This reduces the spread of disease and helps prevent the development of drug-resistant bacterial strains.

### General

Wherever the term ‘comprising’ is used herein we also contemplate options wherein the terms ‘consisting of’ or ‘consisting essentially of’ are used instead. In addition, any and all liquid compositions described herein can be aqueous solutions. Note too that whenever the phrase “one or more” is used for a range, for example in relation to a number of sequences W, X, Y and Z (“one or more of SEQ ID Nos. W, X, Y and Z”) this is a disclosure of each value alone (SEQ ID No. W; SEQ ID No. X; SEQ ID No. Y; SEQ ID No. Z), or in combination, e.g. SEQ ID Nos. W and X and SEQ ID No. Y and Z). Similarly, whenever the phrase “one or more” is used in relation to a range of pairs, for example in relation to a number of pairs of sequences (“one or more of SEQ ID Nos. W and X; and Y and Z”) this is a disclosure of each pair alone (SEQ ID No. W and X) or in combination (e.g. SEQ ID Nos. W and X and SEQ ID Nos. Y and Z).

The following Examples are provided to illustrate embodiments of the present invention and should not be construed as limiting thereof.

Example 1—studies using katG redesigned reverse primer and inhA initial forward primer (SEQ ID NOs: 1-22, 24, 25-32 and 34): references to the katG reverse primer denote the katG redesigned reverse primer (SEQ ID NO: 20); and references to the inhA forward primer denote the inhA initial forward primer (SEQ ID NO: 34). A study was conducted using sputum spiked with well characterized M. tuberculosis isolates (whole-genome sequence and culture confirmed resistance profiles) to evaluate the developed primers and method. DNA was extracted on the MagNA Pure Compact, PCR amplified in 3 multiplex reactions per sample, pooled, washed, barcoded, and sequenced on the MinION in batches of 80 as described below.

### DNA Extraction:

1. In a Microbiological Class II Safety Cabinet (MSC-II) unseal liquid clinical sample and aliquot 750 μL to a fresh 1.5 mL Eppendorf tube with screw cap.
2. In MSC-II load sample Eppendorf tubes into an aerosol-sealable centrifuge rotor.
3. Centrifuge 750 μL clinical sputum sample at 15,000 g for 5 min, after which the centrifuge rotor is returned to the MSC-II and samples removed.
4. In MSC-II carefully remove supernatant and resuspend pellet in 700 μL MagNA Pure Bacterial Lysis Buffer (BLB) [Roche Life Science].
5. In MSC-II transfer 700 μL of resuspended samples to bead-beating tubes with screw cap (Lysing Matrix E tubes from MP Biomedical).
6. In MSC-II bead-beat samples in a FastPrep homogenizer at maximum speed for 45 seconds.
7. Repeat Step 6.
8. In MSC-II load bead-beating tubes into an aerosol-sealable centrifuge rotor.
9. Spin down bead-beating tubes at maximum speed for 2 minutes.
10. Return centrifuge rotor to the MSC-II and gently remove bead-beating tubes.
11. In MSC-II transfer 230 μL clear supernatant in two 200 μL batches to a clean MagNA Pure sample tube. Add 20 μL Proteinase K to sample.
12. In MSC-II incubate samples on heat block for 5 minutes at 65° C. vortexing in the MSC-II every 30 seconds.
13. Transfer incubated samples to MagNA Pure compact and perform automated extraction.
14. On completion of automated extraction return elute tubes to MSC-II for Multiplex PCR preparation.

### Multiplex PCR:

1. Prepare 3 multiplex 10× primer mixes as follows:

Group 1 10x Primer Mix

| Primer | Volume Added (μL) | Final Concentration |
|---|---|---|
| 100 μM eis FW | 10 | 2 μM |
| 100 μM eis RV | 10 | 2 μM |
| 100 μM embB FW | 10 | 2 μM |
| 100 μM embB RV | 10 | 2 μM |
| 100 μM rrs FW | 10 | 2 μM |
| 100 μM rrs RV | 10 | 2 μM |
| 100 μM rv0678 FW | 10 | 2 μM |
| 100 μM rv0678 RV | 10 | 2 μM |
| 100 μM fabG1 FW | 10 | 2 μM |
| 100 μM fabG1 RV | 10 | 2 μM |
| Nuclease-Free H₂O | 400 | |
| Total Volume | 500 | |

Group 2 10x Primer Mix

| Primer Pair | Volume Added (μL) | Final Concentration |
|---|---|---|
| 100 μM gyrA FW | 10 | 2 μM |
| 100 μM gyrA RV | 10 | 2 μM |
| 100 μM rpoB FW | 10 | 2 μM |
| 100 μM rpoB RV | 10 | 2 μM |
| 100 μM ethA FW | 10 | 2 μM |
| 100 μM ethA RV | 10 | 2 μM |
| 100 μM rplC FW | 10 | 2 μM |
| 100 μM rplC RV | 10 | 2 μM |
| 100 μM katG FW | 10 | 2 μM |
| 100 μM katG redesigned RV | 10 | 2 μM |
| Nuclease-Free H₂O | 400 | |
| Total Volume | 500 | |

Group 3 10x Primer Mix

| Primer Pair | Volume Added (μL) | Final Concentration |
|---|---|---|
| 100 μM gidB FW | 10 | 2 μM |
| 100 μM gidB RV | 10 | 2 μM |
| 100 μM inhA initial FW | 10 | 2 μM |
| 100 μM inhA RV | 10 | 2 μM |
| 100 μM rrl FW | 10 | 2 μM |
| 100 μM rrl RV | 10 | 2 μM |
| 100 μM rpsL FW | 10 | 2 μM |
| 100 μM rpsL RV | 10 | 2 μM |
| 100 μM pncA FW | 10 | 2 μM |
| 100 μM pncA RV | 10 | 2 μM |
| 100 μM tlyA FW | 15 | 3 μM |
| 100 μM tlyA RV | 15 | 3 μM |
| Nuclease-Free H₂O | 370 | |
| Total Volume | 500 | |

2. In MSC-II mix PCR Master Mix (Qiagen Multiplex PCR kit) for each multiplex primer group in the following ratio per sample:

| Reagent | Volume per Sample (μL) |
|---|---|
| 2x Qiagen Multiplex Master Mix | 25 |
| 10x Primer Mix | 5 |
| 5x Q-Solution | 10 |
| Nuclease-Free Water | 5 |

3. In MSC-II add 45 μL mastermix to 0.2 mL thin-walled PCR tubes.
   a. Each sample requires three tubes, one for each Multiplex Primer Group.
4. In MSC-II carefully add 5 μL extracted DNA to PCR tubes.
5. In MSC-II seal PCR tubes tightly and vortex.
6. In MSC-II briefly spin down PCR tubes and remove bubbles.
7. Load PCR tubes into a thermocycler and run an amplification protocol with the following parameters:

| Step | Time (mm:ss) | Temperature (° C.) | Cycles |
|---|---|---|---|
| Heat Activation | 20:00 | 95 | 1 |
| Denaturation | 00:30 | 95 | 35 |
| Annealing | 01:30 | 60 | |
| Extension | 01:30 | 72 | |
| Final Extension | 10:00 | 72 | 1 |

8. Carefully remove PCR tubes and return to MSC-II.
9. In MSC-II transfer PCR product to clean PCR tubes.
10. Submerge clean PCR tubes in a 1:16 dilution of Bioguard for minimum 30 seconds for removal from CL3.

The three multiplex reactions for each sample are then pooled as follows:
1. Mix Qubit High Sensitivity assay buffer according to manufacturer specifications for each sample Multiplex Group.
   a. 200 μL Qubit Buffer+1 μL Qubit Dye per sample
2. In a clear flat-bottomed 96-well plate aliquot 198 μL of mixed Qubit solution to each well.
3. Add 2 μL of each multiplex group template so each well has a single template.
4. Analyze plate on a Promega QuantiFlor or similar plate reader.
5. Using quantification results, pool the 3 sample multiplex groups in equimolar concentrations to a total of 1 μg.
   a. In case pooled sample total volume is below 45 μL normalize volume of all samples to 100 μL using Nuclease-Free H₂O
   b. If there is insufficient DNA for a pooled total of 1 μg, equimolar pool at a lower concentration but in a max volume of 100 μl

The pooled samples were then prepared for nanopore sequencing as follows:

### End Prep

1. Transfer 45 μL of pooled DNA to a thin-walled PCR plate
2. Add following reagents to the DNA

| Reagent | Volume per Sample (μL) |
|---|---|
| Template DNA (<1,000 ng) | 45 |
| Ultra II End-Prep Buffer | 7 |
| Ultra II End-Prep Enzyme Mix | 3 |
| Nuclease Free H₂O | 5 |
| Total | 60 |

3. Mix by pipette
4. Spin down tube and incubate for 5 minutes at 20° C. followed by 5 minutes at 65° C.
5. Transfer samples to a clean 96-well plate
6. Perform a 1× bead wash by adding 60 μL AMPure XP Beads
7. Incubate sample for 5 minutes on a hula mixer
8. Briefly spin down plate
9. Place plate on magnet-rack and let incubate for 5 minutes
10. Remove supernatant
11. Wash bead pellet with 180 μL 70% ethanol
12. Remove supernatant
13. Wash bead pellet with 180 μL 70% ethanol
14. Remove supernatant
15. Briefly spin down plate and return to magnet-rack
16. Remove residual supernatant
17. Air dry pellet for approximately 30 seconds
18. Resuspend pellet in 31 μL nuclease free H₂O
19. Incubate samples for 2 minutes at room temperature
20. Return plate to magnet-rack and pellet beads for 2 minutes
21. Carefully remove eluted supernatant and transfer 30 μL to a clean 96-well plate

### Barcode Adapter Ligation

1. In a fresh plate add the following reagents in order per sample.
   a. 15 μL End-Prepped DNA
   b. 10 μL Barcode Adapter (BCA)
   c. 25 μL Blunt/TA Ligase Master Mix
2. Mix by pipetting.
3. Briefly spin down plate.
4. Incubate at room temperature for 10 minutes
5. Perform 0.8× bead wash (30 μL) using AMPure XP beads as described above
6. Resuspend pellet in 25 μL nuclease free H₂O
7. Incubate samples for 2 minutes at room temperature
8. Return plate to magnet-rack and pellet beads for 2 minutes
9. Carefully remove eluted supernatant and transfer to a clean 96-well plate.

### Barcoding PCR

1. In a thin-walled PCR plate combine the following:

| Reagent | Volume per Sample (μL) |
|---|---|
| Adapter Ligated Template DNA | 4 |
| 10 μM PCR Barcode | 1 |
| 2x LongAmp Taq MasterMix | 25 |
| Nuclease Free H₂O | 20 |
| Total | 50 |

2. Briefly vortex
3. Spin down samples
4. PCR amplify using the following cycling conditions

| Cycle Step | Temperature (° C.) | Time (mm:ss) | Cycles |
|---|---|---|---|
| Initial Denaturation | 95 | 03:00 | 1 |
| Denaturation | 95 | 00:15 | 15 |
| Annealing | 62 | 00:15 | |
| Extension | 65 | 01:30 | |
| Final Extension | 65 | 05:00 | 1 |
| Hold | 4 | ∞ | N/A |

5. Perform 0.8× bead wash (40 μL) using AMPure XP beads as described above
6. Resuspend pellet in 45 μL nuclease free H₂O
7. Incubate samples for 2 minutes at room temperature
8. Return plate to magnet-rack and pellet beads for 2 minutes
9. Carefully remove eluted supernatant and transfer to a clean 96-well plate.
10. Quantify as described above
11. Pool each barcoded sample equimolar into a fresh 1.5 mL Eppendorf
12. Perform 0.8× bead wash using AMPure XP beads on pooled samples as described above and resuspend in 45 μL nuclease free H₂O

### DNA End-Prep

1. In a 0.2 mL thin walled PCR tube combine the following:

| Reagent | Volume (μL) |
|---|---|
| Pooled Barcoded DNA (1,000 ng) + | 50 |
| Nuclease Free H₂O | |
| Ultra II End-Prep Buffer | 7 |
| Ultra II End-Prep Enzyme Mix | 3 |
| Total | 60 |

2. Vortex and briefly spin down
3. Incubate for 5 minutes at 20° C. followed by 5 minutes at 65° C.
4. Transfer sample to a clean 1.5 mL Eppendorf
5. Perform a 0.8× bead wash (48 μL) using AMPure XP beads as described above
6. Resuspend pellet in 61 μL nuclease free H₂O
7. Incubate samples for 2 minutes at room temperature
8. Return plate to magnet-rack and pellet beads for 2 minutes
9. Carefully remove eluted supernatant and transfer to a clean 1.5 mL Eppendorf.

### Adapter Ligation:

1. Thaw and spin down Adapter Mix (AMX), T4 Ligase, Ligation Buffer (LNB), and Elution Buffer (EB) (Oxford Nanopore Technologies Ligation Sequencing Kit SQK-LSK109).
2. Place thawed and vortexed reagents on ice
3. Thaw one tube of Short Fragment Buffer (SFB) at room temperature
   a. Vortex and spin down before placing on ice
4. Mix the following in a 1.5 mL Eppendorf in order:

| Reagent | Volume (μL) |
|---|---|
| End-Prepped DNA | 60 |
| Ligation Buffer (LNB) | 25 |
| NEBNext Quick T4 DNA Ligase | 10 |
| Adapter Mix (AMX) | 5 |
| Total | 100 |

5. Gently mix tube by flicking and spin down
6. Incubate for 10 minutes at room temperature
7. Perform a 0.6× bead wash (60 μL) using AMPure XP beads
8. Incubate samples for 5 minutes on a hula mixer
9. Briefly spin down samples
10. Place tube on magnet-rack and let incubate for 5 minutes
11. Remove supernatant
12. Resuspend pellet in 125 μL SFB
13. Place tube on magnet-rack and let incubate for 10 minutes
14. Carefully remove supernatant
15. Resuspend pellet in 125 μL SFB
16. Place tube on magnet-rack and let incubate for 10 minutes
17. Carefully remove supernatant
18. Briefly spin down tube and return to magnet-rack
19. Remove residual supernatant
20. Air dry pellet for approximately 30 seconds
21. Resuspend pellet in 15 μL EB
22. Incubate at room temperature for 10 minutes
23. Place tube on magnet-rack until elute is clear and colourless
24. Carefully remove and retain 15 μL eluted supernatant in clean 1.5 mL Eppendorf
25. Perform Qubit HS Assay on 1 μL elute

### Sequencing Library Loading on MinION

1. Perform MinION loading according to Oxford Nanopore Manufacturer protocols
   a. Load between 100 and 150 fmol of DNA as calculated using the Qubit quantification
      i. fmols can be calculated easily from ng using the following website: http://molbiol.edu.ru/eng/scripts/01_07.html

Resistance to first- and second-line anti-TB drugs was identified using the ONT Epi2Me FastQ TB Resistance Profile pipeline. Wild-type and mutant nucleotides were reported for all drug resistance associated SNP sites detected within the PCR product fastQ sequences. The presence of SNPs in specific target genes indicated resistance to specific anti-TB drugs (Table 8).

This method also allowed for identification of heteroresistance by comparison of the relative number of reads for wild-type compared to the number of reads for mutants (Table 9). Heteroresistance was called when >15% and <80% mutant bases were detected.

TABLE 8
Example drug resistance profile of two samples sequenced using the developed method

| Sample | Ethambutol | Isoniazid | Pyrazinamide | Rifampicin | Streptomycin | Amikacin |
|---|---|---|---|---|---|---|
| 1 | Resistant | Resistant | Susceptible | Resistant | Susceptible | Resistant |
| 2 | Resistant | Resistant | Susceptible | Resistant | Resistant | Susceptible |

| Sample | Bedaquiline | Capreomycin | Ciprofloxacin | Clofazimine | Ethionamide | Kanamycin |
|---|---|---|---|---|---|---|
| 1 | Susceptible | Resistant | Susceptible | Susceptible | Susceptible | Resistant |
| 2 | Susceptible | Susceptible | Susceptible | Susceptible | Susceptible | Susceptible |

| Sample | Linezolid | Moxifloxacin | Ofloxacin | Quinolones |
|---|---|---|---|---|
| 1 | Susceptible | Resistant | Resistant | Resistant |
| 2 | Susceptible | Susceptible | Susceptible | Resistant |

Raw read numbers could also be visualised, providing a more detailed analysis if required (Table 10). These results display the codon or nucleotide location within the annotated gene as well as the number of wild-type or mutant bases recorded at that location.

TABLE 10
Example of raw data provided through Epi2Me analysis for
two sequenced samples

| | Ethambutol | | Ethambutol | |
|---|---|---|---|---|
| | Resistance | Ethambutol | Wild-Type | Ethambutol |
| Sample | SNP | Mutation | Bases | Mutant Bases |
| 1 | embB M306V | ATG -> GTG | 41 | 954 |
| 2 | embB M306I | ATG -> ATA | 45 | 662 |
| | Isoniazid | | Isoniazid | |
| | Resistance | Isoniazid | Wild- | Isoniazid |
| Sample | SNP | Mutation | Type Bases | Mutant Bases |
| 1 | katG S315T | GCT -> GGT | 35 | 2841 |
| | fabG1 T-8A | T -> A | 50 | 2929 |
| 2 | katG S315T | GCT -> GGT | 31 | 529 |
| | Pyrazinamide | | Pyrazinamide | |
| | Resistance | Pyrazinamide | Wild-Type | Pyrazinamide |
| Sample | SNP | Mutation | Bases | Mutant Bases |
| 1 | N/A | N/A | N/A | N/A |
| 2 | pncA V139A | CAC -> CGC | 865 | 507 |
| | Rifampicin | | Rifampicin | Rifampicin |
| | Resistance | Rifampicin | Wild-Type | Mutant |
| Sample | SNP | Mutation | Bases | Bases |
| 1 | rpoB D435G, | GAC -> GGC | 148 | 1895 |
| | rpoB L452P | CTG -> CCG | 73 | 1629 |
| 2 | rpoB H445N, | CAC -> AAC | 1396 | 1060 |
| | rpoB D435S | GAC -> TCC | 1161 | 1385 |
| | (double | GAC -> TCC | 1462 | 758 |
| | mutation) | | | |
| | Streptomycin | | Streptomycin | Streptomycin |
| | Resistance | Streptomycin | Wild-Type | Mutant |
| Sample | SNP | Mutation | Bases | Bases |
| 1 | N/A | N/A | N/A | N/A |
| 2 | gidB A205E | TGC -> CGC | 18 | 1737 |
| | rpsL K43R | AAG -> AGG | 52 | 294 |
| | Amikacin | | Amikacin | |
| | Resistance | Amikacin | Wild- | Amikacin |
| Sample | SNP | Mutation | Type Bases | Mutant Bases |
| 1 | rrs A1401G | A -> G | 27 | 2908 |
| 2 | N/A | N/A | N/A | N/A |
| | Capreomycin | | Capreomycin | Capreomycin |
| | Resistance | Capreomycin | Wild-Type | Mutant |
| Sample | SNP | Mutation | Bases | Bases |
| 1 | rrs A1401G | A -> G | 27 | 2908 |
| 2 | N/A | N/A | N/A | N/A |
| | Ciprofloxacin | | Ciprofloxacin | Ciprofloxacin |
| | Resistance | Ciprofloxacin | Wild-Type | Mutant |
| Sample | SNP | Mutation | Bases | Bases |
| 1 | N/A | N/A | N/A | N/A |
| 2 | gyrA D94G | GAC -> GGC | 3347 | 2004 |
| | Ethionamide | | Ethionamide | |
| | Resistance | Ethionamide | Wild-Type | Ethionamide |
| Sample | SNP | Mutation | Bases | Mutant Bases |
| 1 | N/A | N/A | N/A | N/A |
| 2 | N/A | N/A | N/A | N/A |
| | Kanamycin | | Kanamycin | |
| | Resistance | Kanamycin | Wild-Type | Kanamycin |
| Sample | SNP | Mutation | Bases | Mutant Bases |
| 1 | rrs A1401G | A -> G | 27 | 2908 |
| 2 | N/A | N/A | N/A | N/A |
| | Moxifloxacin | | Moxifloxacin | |
| | Resistance | Moxifloxacin | Wild-Type | Moxifloxacin |
| Sample | SNP | Mutation | Bases | Mutant Bases |
| 1 | gyrA A90V | GCG -> GTG | 331 | 3644 |
| 2 | gyrA D94G | GAC -> GGC | 3347 | 2004 |
| | Ofloxacin | | Ofloxacin | Ofloxacin |
| | Resistance | Ofloxacin | Wild- | Mutant |
| Sample | SNP | Mutation | Type Bases | Bases |
| 1 | gyrA A90V | GCG -> GTG | 331 | 3644 |
| 2 | gyrA D94G | GAC -> GGC | 3347 | 2004 |
| | Quinolones | | Quinolones | |
| | Resistance | Quinolones | Wild-Type | Quinolones |
| Sample | SNP | Mutation | Bases | Mutant Bases |
| 1 | gyrA A90V | GCG -> GTG | 331 | 3644 |
| | gyrA D94G | GAC -> GGC | 3347 | 2004 |
| 2 | gyrA D89N | GAC -> AAC | 2338 | 3506 |

Example 2—studies using katG redesigned reverse primer and inhA initial forward primer (SEQ ID NOs: 1-22, 24, 25-32 and 34): references to the katG reverse primer denote the katG redesigned reverse primer (SEQ ID NO: 20); and references to the inhA forward primer denote the inhA initial forward primer (SEQ ID NO: 34).

Following on from Example 1, a set of samples were processed with an altered DNA extraction and simplified library preparation method. Here, DNA was extracted instead using the Promega Maxwell RSC 48 with the PureFood Pathogen kit and within the library preparation alterations were made to the end-prep and barcode/adapter ligation reactions. The resistance profile was compared between methods to ensure the same profile was identified. Details of the method alterations are below:

### DNA Extraction:

1. In a Microbiological Class II Safety Cabinet (MSC-II) in the level 3 containment facility (CL3) unseal liquid clinical sample and aliquot 750 μL to a fresh 1.5 mL Eppendorf tube with screw cap.
2. In MSC-II load sample Eppendorf tubes into an aerosol-sealable centrifuge rotor.
3. Centrifuge 750 μL clinical sputum sample at 15,000×g for 5 min, after which the centrifuge rotor is returned to the MSC-II and samples removed.
4. In MSC-II carefully remove supernatant and resuspend pellet in 700 μL Phosphate Buffered Saline (PBS).
5. In MSC-II transfer 700 μL of resuspended samples to bead-beating tubes with screw cap (Lysing Matrix E tubes from MP Biomedical).
6. In MSC-II bead-beat samples in a FastPrep-24 homogenizer at maximum speed for 45 seconds.
7. Repeat Step 6.
8. In MSC-II load bead-beating tubes into an aerosol-sealable centrifuge rotor.
9. Spin down bead-beating tubes at maximum speed for 3 minutes.
10. Return centrifuge rotor to the MSC-II and gently remove bead-beating tubes.
11. In MSC-II transfer 400 μL clear supernatant in two 200 μL aliquots to a clean 2 ml screw-capped sample tube. Add 40 μL Proteinase K to sample.
12. In MSC-II add 200 μL of Lysis Buffer A from the Maxwell RSC PureFood Pathogen Kit [Promega]
13. In MSC-II incubate samples on heat block for 10 minutes at 65° C. vortexing in the MSC-II every 30 seconds.
14. In MSC-II add 400 μL PBS and 300 μL Lysis Buffer from the Maxwell RSC PureFood Pathogen Kit [Promega]
15. Transfer samples to the Maxwell RSC sample well and prepare the automated extraction according to manufacturer instructions.
16. When automated extraction is completed return elution tubes to MSC-II for Multiplex PCR Preparation.

### End Prep

1. Transfer 12.5 μL (<450 ng) of pooled DNA to a thin-walled PCR plate
2. Add following reagents to the DNA

| Reagent | Volume per Sample (μL) |
|---|---|
| Ultra II End-Prep Buffer | 1.75 |
| Ultra II End-Prep Enzyme Mix | 0.75 |
| Total with DNA | 15 |

3. Mix by pipette
4. Spin down tube and incubate for 5 minutes at 20° C. followed by 5 minutes at 65° C.

### Barcode Ligation

5. In a fresh 96-well plate add the following reagents in order per sample.
   a. 3 μL Nuclease-Free H₂O
   b. 0.75 μL End-Prepped DNA
   c. 1.25 μL Native Barcode (1 per Sample)
   d. 5 μL Blunt/TA Ligase Master Mix
6. Mix by pipetting and briefly spin down plate.
7. Incubate for 20 minutes at 20° C. followed by 10 minutes at 65° C.
8. Pool all samples in a clean 1.5 mL Eppendorf and carry 480 μL forward
   e. If pooled volume is <480 μL use total volume instead
9. Perform a 0.4× Bead Wash
   f. 192 μL of resuspended AMPure XP Beads for 480 μL of pooled sample
10. Incubate samples for 10 minutes at room temperature on a Hula Mixer
11. Place the sample on a magnet rack and incubate for 5 minutes
12. Carefully remove the supernatant and resuspend the bead pellet in 700 μL Short Fragment Buffer (SFB) [Oxford Nanopore]
13. Return the sample to the magnet rack and incubate for 5 minutes
14. Repeat steps 12 and 13
15. Carefully remove the supernatant and, leaving the tube on the magnet rack, wash the bead pellet with 100 μL 70% ethanol
16. Remove the supernatant and briefly spin down the tube before replacing it on the magnet rack
17. Using a p10 remove any residual supernatant and allow the pellet to air dry for approximately 30 seconds
   a. Take care not to let the pellet crack
18. Resuspend the pellet in 35 μL of nuclease-free H₂O and incubate for 2 minutes at room temperature
19. Return the tube to the magnet rack and incubate for 2 minutes, carefully transfer 35 μL of supernatant to a clean Eppendorf.

### Adapter Ligation:

20. Thaw and spin down Adapter Mix (AMII) [ONT], Quick Ligation Reaction Buffer [NEB], Quick T4 Ligase [NEB], and Elution Buffer (EB) [ONT], and SFB [ONT]
21. Place thawed and vortexed reagents on ice
22. Mix the following in a 1.5 mL Eppendorf in order:

| Reagent | Volume (μL) |
|---|---|
| End-Prepped DNA | 30 |
| Quick Ligation Reaction Buffer | 10 |
| NEBNext Quick T4 DNA Ligase | 5 |
| Adapter Mix (AMII) | 5 |
| Total | 50 |

23. Gently mix tube by flicking and spin down
24. Incubate for 20 minutes at room temperature
25. Perform a 0.4× bead wash (20 μL) using resuspended AMPure XP beads
26. Incubate samples for 10 minutes on a hula mixer
27. Briefly spin down samples and place tube on magnet-rack and let incubate for 5 minutes
28. Carefully remove supernatant and resuspend the pellet in 125 μL SFB
29. Place tube on magnet-rack and let incubate for 5 minutes
30. Repeat steps 28 and 29
31. Briefly spin down tube and return to magnet-rack
32. Using a p10 remove residual supernatant
33. Air dry pellet for approximately 30 seconds
   a. Take care not to let the pellet crack
34. Resuspend pellet in 15 μL EB and incubate at room temperature for 10 minutes
35. Place tube on magnet-rack until elute is clear and colourless
36. Carefully remove and retain 15 μL eluted supernatant in clean 1.5 mL Eppendorf
37. Perform Qubit HS Assay on 1 μL elute.

Resistance to ‘first- and second-line anti-TB drugs was identified using the ONT Epi2Me FastQ TB Resistance Profile pipeline. Wild-type and mutant nucleotides were reported for all drug resistance associated SNP sites detected within the PCR product fastQ sequences. The presence of SNPs (>15% mutant bases) in specific target genes indicated resistance to specific anti-TB drugs (Table 11).

TABLE 11
Example drug resistance profile of two samples sequenced using the developed method

| Sample | Ethambutol | Isoniazid | Pyrazinamide | Rifampicin | Streptomycin | Amikacin |
|---|---|---|---|---|---|---|
| 1 | Resistant | Resistant | Susceptible | Resistant | Susceptible | Resistant |
| 2 | Resistant | Resistant | Susceptible | Resistant | Resistant | Susceptible |

| Sample | Bedaquiline | Capreomycin | Ciprofloxacin | Clofazimine | Ethionamide | Kanamycin |
|---|---|---|---|---|---|---|
| 1 | Susceptible | Resistant | Susceptible | Susceptible | Susceptible | Resistant |
| 2 | Susceptible | Susceptible | Susceptible | Susceptible | Susceptible | Susceptible |

| Sample | Linezolid | Moxifloxacin | Ofloxacin | Quinolones |
|---|---|---|---|---|
| 1 | Susceptible | Resistant | Resistant | Resistant |
| 2 | Susceptible | Susceptible | Susceptible | Resistant |

Raw read numbers could also be visualised, providing a more detailed analysis if required (Table 12) e.g. for identifying heteroresistance. These results display the codon or nucleotide location within the annotated gene as well as the number of wild-type or mutant bases recorded at that location.

TABLE 12
Example of raw data provided through Epi2Me analysis for
two sequenced samples

| | Ethambutol | | Ethambutol | |
|---|---|---|---|---|
| | Resistance | Ethambutol | Wild-Type | Ethambutol |
| Sample | SNP | Mutation | Bases | Mutant Bases |
| 1 | embB M306V | ATG -> GTG | 41 | 954 |
| 2 | embB M306I | ATG -> ATA | 45 | 662 |
| | Isoniazid | | Isoniazid | Isoniazid |
| | Resistance | Isoniazid | Wild- | Mutant |
| Sample | SNP | Mutation | Type Bases | Bases |
| 1 | katG S315T | GCT -> GGT | 35 | 2841 |
| | fabG1 T-8A | T -> A | 50 | 2929 |
| 2 | katG S315T | GCT -> GGT | 31 | 529 |
| | Pyrazinamide | | Pyrazinamide | Pyrazinamide |
| | Resistance | Pyrazinamide | Wild-Type | Mutant |
| Sample | SNP | Mutation | Bases | Bases |
| 1 | N/A | N/A | N/A | N/A |
| 2 | pncA V139A | CAC -> CGC | 865 | 507 |
| | Rifampicin | | Rifampicin | Rifampicin |
| | Resistance | Rifampicin | Wild- | Mutant |
| Sample | SNP | Mutation | Type Bases | Bases |
| 1 | rpoB D435G, | GAC -> GGC | 148 | 1895 |
| | rpoB | | | |
| | L452P | CTG -> CCG | 73 | 1629 |
| 2 | rpoB H445N, | CAC -> AAC | 1396 | 1060 |
| | rpoB | | | |
| | D435S (double | GAC -> TCC | 1161 | 1385 |
| | mutation) | GAC -> TCC | 1462 | 758 |
| | Streptomycin | | Streptomycin | |
| | Resistance | Streptomycin | Wild-Type | Streptomycin |
| Sample | SNP | Mutation | Bases | Mutant Bases |
| 1 | N/A | N/A | N/A | N/A |
| 2 | gidB A205E | TGC -> CGC | 18 | 1737 |
| | rpsL K43R | AAG -> AGG | 52 | 294 |
| | Amikacin | | Amikacin | |
| | Resistance | Amikacin | Wild- | Amikacin |
| Sample | SNP | Mutation | Type Bases | Mutant Bases |
| 1 | rrs A1401G | A -> G | 27 | 2908 |
| 2 | N/A | N/A | N/A | N/A |
| | Capreomycin | | Capreomycin | |
| | Resistance | Capreomycin | Wild-Type | Capreomycin |
| Sample | SNP | Mutation | Bases | Mutant Bases |
| 1 | rrs A1401G | A-> G | 27 | 2908 |
| 2 | N/A | N/A | N/A | N/A |
| | Ciprofloxacin | | Ciprofloxacin | Ciprofloxacin |
| | Resistance | Ciprofloxacin | Wild-Type | Mutant |
| Sample | SNP | Mutation | Bases | Bases |
| 1 | N/A | N/A | N/A | N/A |
| 2 | gyrA D94G | GAC -> GGC | 3347 | 2004 |
| | Ethionamide | | Ethionamide | |
| | Resistance | Ethionamide | Wild-Type | Ethionamide |
| Sample | SNP | Mutation | Bases | Mutant Bases |
| 1 | N/A | N/A | N/A | N/A |
| 2 | N/A | N/A | N/A | N/A |
| | Kanamycin | | Kanamycin | |
| | Resistance | Kanamycin | Wild- | Kanamycin |
| Sample | SNP | Mutation | Type Bases | Mutant Bases |
| 1 | rrs A1401G | A -> G | 27 | 2908 |
| 2 | N/A | N/A | N/A | N/A |
| | Moxifloxacin | | Moxifloxacin | |
| | Resistance | Moxifloxacin | Wild-Type | Moxifloxacin |
| Sample | SNP | Mutation | Bases | Mutant Bases |
| 1 | gyrA A90V | GCG -> GTG | 331 | 3644 |
| 2 | gyrA D94G | GAC -> GGC | 3347 | 2004 |
| | Ofloxacin | | Ofloxacin | |
| | Resistance | Ofloxacin | Wild- | Ofloxacin |
| Sample | SNP | Mutation | Type Bases | Mutant Bases |
| 1 | gyrA A90V | GCG -> GTG | 331 | 3644 |
| 2 | gyrA D94G | GAC -> GGC | 3347 | 2004 |
| | Quinolones | | Quinolones | Quinolones |
| | Resistance | Quinolones | Wild-Type | Mutant |
| Sample | SNP | Mutation | Bases | Bases |
| 1 | gyrA A90V | GCG -> GTG | 331 | 3644 |
| 2 | gyrA D94G | GAC -> GGC | 3347 | 2004 |
| | gyrA D89N | GAC -> AAC | 2338 | 3506 |

As can be seen from both results tables the alterations in methodology did not change the resistance profile of this sample. Therefore the optimised method (using the Promega Maxwell and simplified library preparation) would be the method of choice for this assay.

TABLE 13
Drug resistance profile of a sample sequenced using
method 1 (Example 1) and 2 (Example 2)

| | Resistance call | |
|---|---|---|
| Drug | Method 1 | Method 2 |
| Ethambutol | Resistant | Resistant |
| Isoniazid | Resistant | Resistant |
| Pyrazinamide | Resistant | Resistant |
| Rifampicin | Resistant | Resistant |
| Streptomycin | Resistant | Resistant |
| Amikacin | Susceptible | Susceptible |
| Capreomycin | Susceptible | Susceptible |
| Bedaquiline | Susceptible | Susceptible |
| Ciprofloxacin | Susceptible | Susceptible |
| Clofazamine | Susceptible | Susceptible |
| Ethionamide | Susceptible | Susceptible |
| Kanamycin | Susceptible | Susceptible |
| Linezolid | Susceptible | Susceptible |
| Moxifloxacin | Susceptible | Susceptible |
| Ofloxacin | Susceptible | Susceptible |
| Quinolones | Susceptible | Susceptible |

TABLE 14
Example of raw data provided through Epi2Me analysis for a sample
comparing methods 1 (Example 1) and 2 (Example 2).

| | Ethambutol | | Ethambutol | Ethambutol |
|---|---|---|---|---|
| | Resistance | Ethambutol | Wild-Type | Mutant |
| Sample | SNP | Mutation | Bases | Bases |
| Method | embB G406D | GGC -> GAC | 115 | 303 |
| 1 | embB E378A | GAG -> GCG | 23 | 379 |
| Method | embB G406D | GGC -> GAC | 219 | 1684 |
| 2 | embB E378A | GAG -> GCG | 20 | 1814 |
| | embB S347I | AGT -> GGT | 1004 | 306 |
| | Isoniazid | | Isoniazid | Isoniazid |
| | Resistance | Isoniazid | Wild- | Mutant |
| Sample | SNP | Mutation | Type Bases | Bases |
| Method | katG S315T | GCT -> GGT | 8 | 281 |
| 1 | fabG1 C-15T | C->T | 38 | 1604 |
| Method | katG S315T | GCT -> GGT | 51 | 5440 |
| 2 | fabG1 C-15T | C-> T | 12 | 2526 |
| | Pyrazinamide | | Pyrazinamide | Pyrazinamide |
| | Resistance | Pyrazinamide | Wild-Type | Mutant |
| Sample | SNP | Mutation | Bases | Bases |
| Method | pncA C14. | GCA -> TCA | 42 | 737 |
| 1 | | | | |
| Method | | GCA -> TCA | 66 | |
| 2 | pncA C14. | | | 3208 |
| | Rifampicin | | Rifampicin | Rifampicin |
| | Resistance | Rifampicin | Wild- | Mutant |
| Sample | SNP | Mutation | Type Bases | Bases |
| Method | rpoB H445C | CAC -> TGC | 248 | 1378 |
| 1 | (double | | 141 | 1407 |
| | mutation) | | | |
| Method | rpoB H445C | CAC -> TGC | 298 | 1613 |
| 2 | (double | | 144 | 2628 |
| | mutation) | | | |
| | Streptomycin | | Streptomycin | Streptomycin |
| | Resistance | Streptomycin | Wild-Type | Mutant |
| Sample | SNP | Mutation | Bases | Bases |
| Method | gidB A205E | TGC -> CGC | 17 | 888 |
| 1 | | | | |
| Method | gidB A205E | TGC -> CGC | 28 | 3311 |
| 2 | | | | |

### Example 3—Single Multiplex Reaction Including inhA Redesigned Forward Primer inhA FW 6 (SEQ ID Nos: 1-32)

Working primer stocks were prepared as follows:

| | Volume | Final |
|---|---|---|
| Primers at 100 μM | Added | Concentration (μM) |
| eis Forward | 20 | 3 |
| eis Reverse | 20 | 3 |
| embB Forward | 20 | 3 |
| embB Reverse | 20 | 3 |
| rrs Forward | 20 | 3 |
| rrs Reverse | 20 | 3 |
| rv0678 Forward | 20 | 3 |
| rv0678 Reverse | 20 | 3 |
| fabG1 Forward | 20 | 3 |
| fabG1 Reverse | 20 | 3 |
| gyrA Forward | 20 | 3 |
| gyrA Reverse | 20 | 3 |
| rpoB Forward | 20 | 3 |
| rpoB Reverse | 20 | 3 |
| ethA Forward | 20 | 3 |
| ethA Reverse | 20 | 3 |
| rplC Forward | 20 | 3 |
| rplC Reverse | 20 | 3 |
| katG Forward | 20 | 3 |
| katG redesigned Reverse | 20 | 3 |
| gidB Forward | 20 | 3 |
| gidB Reverse | 20 | 3 |
| inhA redesigned Forward | 20 | 3 |
| inhA FW6 | | |
| inhA Reverse | 20 | 3 |
| rrl Forward | 20 | 3 |
| rrl Reverse | 20 | 3 |
| pncA Forward | 20 | 3 |
| pncA Reverse | 20 | 3 |
| rpsL Forward | 20 | 3 |
| rpsL Reverse | 20 | 3 |
| tlyA Forward | 30 | 4.5 |
| tlyA Reverse | 30 | 4.5 |
| Nuclease-Free H₂O | 6.7 | |
| Total Volume | 666.7 | |

1. A PCR master mix was prepared (Qiagen Multiplex PCR kit 206145)

| | Volume per | Volume for a 24 sample |
|---|---|---|
| | Sample | mastermix per |
| Reagent | (μL) | multiplex (μL) |
| 2x Qiagen Multiplex Master Mix | 25 | 660 |
| Primer Mix (3 μM) | 6.7 | 176.9 |
| DMSO | 1 | 26.4 |
| 5x Q-Solution | 10 | 264 |
| Nuclease-Free Water | 2.3 | 60.7 |
| Total volume | 45 | |

2. 45 μl of master mix was aliquoted per PCR reaction and 5 μl DNA template added, followed by vortexing and briefly spinning down. At this stage the positive control was included as a sample alongside a PCR negative control (5 μl nuclease-free water).
3. PCR cycle conditions:

| Step | Time (mm:ss) | Temperature | Cycles |
|---|---|---|---|
| Heat Activation | 20:00 | 95 | 1 |
| Denaturation | 00:30 | 95 | 35 |
| Annealing | 01:30 | 63 | |
| Extension | 01:30 | 72 | |
| Final Extension | 10:00 | 72 | 1 |

Quantification after Multiplex PCR
1. Using 1× dsDNA broad range qubit reagents aliquot 198 μl per sample and 2×190 μl for each standard;
2. Add 10 μl of each standard to 190 μl qubit reagent;
3. Add 2 μl of pooled PCR products to 198 μl qubit reagent;
4. Vortex for 4-5 s then incubate in the dark at RT for 2 min;
5. Read on the Qubit;
6. Subtract the concentration for the PCR negative control from all samples (excluding the positive control);
7. Using this calculated concentration, dilute the samples to 10 ng/μl (if the concentration is lower than 10 ng/μl proceed with 12.5 μl into the end prep reaction). If a sample quantified below the PCR negative control, 12.5 μl of sample was still be processed.

### REFERENCES

1. Coscolla M, Gagneux S. Seminars in Immunology Consequences of genomic diversity in Mycobacterium tuberculosis. Semin. Immunol. 2014; 26(6):431-444. Available at: http://dx.doi.org/10.1016/j.smim.2014.09.012.
2. Doughty E L, Sergeant M J, Adetifa I, Antonio M, Pallen M J. Culture-independent detection and characterisation of Mycobacterium tuberculosis and M. africanum in sputum samples using shotgun metagenomics on a benchtop sequencer. PeerJ. 2014; 2:1-18.
3. Chatterjee A, Nilgiriwala K, Saranath D, Rodrigues C, Mistry N. Whole genome sequencing of clinical strains of Mycobacterium tuberculosis from Mumbai, India: A potential tool for determining drug-resistance and strain lineage. Tuberculosis. 2017; 107:63-72. Available at: https://doi.org/10.1016/j.tube.2017.08.002.
4. Costa P, Botelho A, Couto I, Viveiros M, Inácio J. Standing of nucleic acid testing strategies in veterinary diagnosis laboratories to uncover Mycobacterium tuberculosis complex members. Front. Mol. Biosci. 2014; 1(October):1-11.
5. Gupta S, Kakkar V. Biosensors and Bioelectronics Recent technological advancements in tuberculosis diagnostics—A review. Biosens. Bioelectron. 2018; 115(May):14-29. Available at: https://doi.org/10.1016/j.bios.2018.05.017.
6. Wlodarska M, Johnston J C, Gardy J L. A Microbiological Revolution Meets an Ancient Disease: Improving the Management of Tuberculosis with Genomics. 2015; 28(2):523-539.
7. Jagielski T, Minias A, Ingen J Van, Rastogi N, Brzostek A. Methodological and Clinical Aspects of the Molecular Epidemiology of Mycobacterium tuberculosis and Other Mycobacteria. Clin. Microbiol. Rev. 2016; 29(2):239-290.
8. N'Dira Sanoussi C, Affolabi D, Rigouts L, Anagonou S, Jong B de. Genotypic characterization directly applied to sputum improves the detection of Mycobacterium africanum West African 1, under-represented in positive cultures. PLoS Negl. Trop. Dis. 2017:1-13.
9. Rue-albrecht K, Magee D A, Killick K E, et al. Comparative functional genomics and the bovine macrophage response to strains of the Mycobacterium genus. Front. Immunol. 2014; 5(November):1-14.
10. Ingen J Van, Rahim Z, Mulder A, et al. Characterization of Mycobacterium orygis as M tuberculosis Complex Subspecies. Emerg. Infect. Dis. 2012; 18(4):653-655.
11. Dippenaar A, David S, Parsons C, et al. Whole genome sequence analysis of Mycobacterium suricattae. Tuberculosis. 2015; 95(6):682-688. Available at: http://dx.doi.org/10.1016/j.tube.2015.10.001.
12. Alexander K A, Laver P N, Williams M C, et al. Pathology of the Emerging Mycobacterium tuberculosis Complex Pathogen, Mycobacterium mungi, in the Banded Mongoose (Mungos mungo). 2018; 55(2):303-309.
13. Guthrie J L, Gardy J L. A brief primer on genomic epidemiology: lessons learned from Mycobacterium tuberculosis. Ann. N. Y. Acad. Sci. 2016:59-78.
14. Mcnerney R, Clark T G, Campino S, et al. International Journal of Infectious Diseases Removing the bottleneck in whole genome sequencing of Mycobacterium tuberculosis for rapid drug resistance analysis: a call to action. Int. J. Infect. Dis. 2017; 56:130-135. Available at: http://dx.doi.org/10.1016/j.ijid.2016.11.422.
15. Pankhurst L J, Elias O, Votintseva A A, et al. Rapid, comprehensive, and affordable mycobacterial diagnosis with whole-genome sequencing: a prospective study. Lancet Respir. 4(1):49-58. Available at: http://dx.doi.org/10.1016/S2213-2600(15)00466-X.
16. Brown A C, Bryant J M, Einer-jensen K, et al. Rapid Whole-Genome Sequencing of Mycobacterium tuberculosis Isolates Directly from Clinical Samples. J. Clin. Microbiol. 2015; 53(7):2230-2237.
17. Kulchavenya E. Extrapulmonary tuberculosis: are statistical reports accurate? Ther. Adv. Infect. Dis. 2014; 2(2):61-70.
18. Fisher M, Dolby T, Surtie S, et al. Improved method for collection of sputum for tuberculosis testing to ensure adequate sample volumes for molecular diagnostic testing. J. Microbiol. Methods. 2017; 135:35-40. Available at: http://dx.doi.org/10.1016/j.mimet.2017.01.011.
19. World Health Organization. Global Tuberculosis Report. 2019.
20. Quan T P, Bawa Z, Foster D, et al. Evaluation of Whole-Genome Sequencing for Mycobacterial Species Identification and Drug Susceptibility Testing in a Clinical Setting: a Large-Scale Prospective Assessment of Performance against Line Probe Assays and Phenotyping. J. Chromatogr. B Anal. Technol. Biomed. Life Sci. 2018; 56(2):1-14.
21. Zumla A, Al-Tawfiq J A, Enne V I, et al. Rapid point of care diagnostic tests for viral and bacterial respiratory tract infections-needs, advances, and future prospects. Lancet Infect. Dis. 2014; 14(11):1123-1135.
22. Walker T M, Kohl T A, Omar S V, et al. Whole-genome sequencing for prediction of Mycobacterium tuberculosis drug susceptibility and resistance: a retrospective cohort study. Lancet Infect. Dis. 2015; 15:1193-1202.
23. Gardy J L. Towards genomic prediction of drug resistance in tuberculosis. Lancet Infect. Dis. 2015; 15(10):1124-1125. Available at: http://dx.doi.org/10.1016/S1473-3099(15)00088-2.
24. Bradley P, Gordon N C, Walker T M, et al. Rapid antibiotic-resistance predictions from genome sequence data for Staphylococcus aureus and Mycobacterium tuberculosis. Nat. Commun. 2015; 6:1-14. Available at: http://dx.doi.org/10.1038/ncomms10063.
25. Papaventsis D, Casali N, Kontsevaya I, et al. Whole genome sequencing of Mycobacterium tuberculosis for detection of drug resistance: a systematic review. Clin. Microbiol. Infect. 2017; 23(2):61-68. Available at: http://dx.doi.org/10.1016/j.cmi.2016.09.008.
26. Nimmo C, Doyle R, Burgess C, et al. International Journal of Infectious Diseases Rapid identification of a Mycobacterium tuberculosis full genetic drug resistance profile through whole genome sequencing directly from sputum. Int. J. Infect. Dis. 2017; 62:44-46. Available at: http://dx.doi.org/10.1016/j.ijid.2017.07.007.
27. Linger Y, Knickerbocker C, Sipes D, et al. Genotyping Multidrug-Resistant Mycobacterium tuberculosis from Primary Sputum and Decontaminated Sediment with an Integrated Microfluidic Amplification Microarray Test. J. Clin. Microbiol. 2018; 56(3):1-11.
28. Miotto P, Tessema B, Tagliani E, et al. A standardised method for interpreting the association between mutations and phenotypic drug resistance in Mycobacterium tuberculosis. Eur. Respir. J. 2017; 50. Available at: http://dx.doi.org/10.1183/13993003.01354-2017.
29. World Health Organization. The use of next-generation sequencing technologies for the detection of mutations associated with drug resistance in Mycobacterium tuberculosis complex: technical guide. 2018.
30. Votintseva A A, Bradley P, Pankhurst L J, et al. Same-Day Diagnostic and Surveillance Data for Tuberculosis via Whole-Genome Sequencing of Direct Respiratory Samples. J. Clin. Microbiol. 2017; 55(5):1285-1298.
31. Haas C T, Roe J K, Pollara G, Mehta M, Noursadeghi M. Diagnostic ‘omics’ for active tuberculosis. BMC Med. 2016. Available at: http://dx.doi.org/10.1186/s12916-016-0583-9.
32. Lee R S, Pai M. Real-Time Sequencing of Mycobacterium tuberculosis: Are We There Yet? J. Clin. Microbiol. 2017; 55(5):1249-1254.
33. Allahyartorkaman M, Mirsaeidi M, Hamzehloo G, et al. Low diagnostic accuracy of Xpert MTB/RIF assay for extrapulmonary tuberculosis: A multicenter surveillance. Sci. Rep. 2019; 9:1-6. Available at: http://dx.doi.org/10.1038/s41598-019-55112-y.
34. Jouet A, Gaudin C, Badalato N, et al. free prediction of susceptibility or resistance to 13 anti-tuberculous drugs. Eur. Respir. J. 2020; (June 2020). Available at: http://dx.doi.org/10.1183/13993003.02338-2020.
35. Feuerriegel S, Kohl T A, Utpatel C, et al. Early View Rapid genomic first- and second-line drug resistance prediction from clinical Mycobacterium tuberculosis specimens using Deeplex R-MycTB. Eur. Respir. J. 2020.
36. World Health Organization. The Use of Next-Generation Sequencing Technologies for the Detection of Mutations Associated with Drug Resistance in Mycobacterium tuberculosis Complex: Technical Guide. 2018.
37. Meier A, Kirschner P, Bange F C, Vogel U, Bottger E C. Genetic alterations in streptomycin-resistant Mycobacterium tuberculosis: Mapping of mutations conferring resistance. Antimicrob. Agents Chemother. 1994; 38(2):228-233.
38. Karimi, S., Mirhendi, H., Zaniani F., Manesh, S., Salehi, M., Esfahani B. Rapid detection of streptomycin-resistant Mycobacterium tuberculosis by rpsL-restriction fragment length polymorphism. Adv. Biomed. Res. 2017; 6(126).
39. Villellas C, Aristimuño L, Vitoria M A, et al. Analysis of mutations in streptomycin-resistant strains reveals a simple and reliable genetic marker for identification of the Mycobacterium tuberculosis Beijing genotype. J. Clin. Microbiol. 2013; 51(7):2124-2130.
40. Morlock G P, Metchock B, Sikes D, Crawford J T, Cooksey R C. ethA, inhA and katG Loci of ethionamide-resistant Clinical MTB isolates. Antimicrob. Agents Chemother. 2003; 47(12):3799-3805.
41. Zhao L, Sun Q, Liu H, et al. Analysis of embCAB Mutations Associated with Ethambutol Resistance in Multidrug-Resistant Mycobacterium tuberculosis Isolates from China. Antimicrob. Agents Chemother. 2015; 59(4):2045-2050.
42. Maus C E, Plikaytis B B, Shinnick T M. Mutation of tlyA confers capreomycin resistance in Mycobacterium tuberculosis. Antimicrob. Agents Chemother. 2005; 49(2):571-7. Available at: http://www.ncbi.nlm.nih.gov/pubmed/15673735%0Ahttp://www.pubmedcentral.nih.gov/articlerender.fcgi?artid=PMC547314.
43. (NCBI) NC for BI. Mycobacterium tuberculosis. Available at: https://www.ncbi.nlm.nih.gov/genome/?term=h37rv [Accessed Jul. 17, 2020].

TABLE 5
Optimisation testing results for primer design versions 1-48 in Multiplex measured by nested qPCR

| Multiplex Primer | eis | embB | fabG1 | [symbol] | rv0678 | ethA | gyrA | rpoB | rplC | katG | hsp [symbol] | pncA | inhA | gidB | tlyA | rpsL | [symbol] |
|---|---|---|---|---|---|---|---|---|---|---|---|---|---|---|---|---|---|
| Design Version | CT | CT | CT | CT | CT | CT | CT | CT | CT | CT | CT | CT | CT | CT | CT | CT | CT |
| 1 [symbol] | 6 | 15.57 | 5 | 5 | 6 | 5 | [symbol] | 5 | [symbol] | [symbol] | N/A | [symbol] | 5 | 5 | 5 | [symbol] | [symbol] |
| 2 | 9.73 | 19.77 | [symbol] | 9.27 | 9.98 | [symbol] | 10.56 | 9.31 | 9.8 | [symbol] | N/A | [symbol] | [symbol] | 10.34 | 8.99 | 10.04 | 9.09 |
| 3 | 15.12 | 22.75 | 10.55 | 8.91 | 7.76 | 7.38 | 8.1 | [symbol] | 7.26 | 7.77 | 9.13 | 8.5 | 8.19 | 7.99 | 7.22 | 8.95 | 7.91 |
| 4 | 17.43 | 14.86 | 11.49 | 13.75 | [symbol] | 8.01 | 11.06 | 10.52 | 10.42 | 8.6 | 9.22 | 9.65 | [symbol] | [symbol] | 12.39 | 10.71 | 8.4 |
| 5 | 18.94 | 19.77 | 9.8 | 11.76 | 10.93 | 9.35 | 9.23 | 10.22 | [symbol] | 10.99 | 10.86 | 10.85 | 10.02 | [symbol] | 10.53 | [symbol] | 9.93 |
| 6 | 17.73 | 24.28 | 10.63 | 10.95 | 8.84 | 7.7 | 10.64 | 10.88 | [symbol] | 10.61 | 11.67 | 11.26 | 9.62 | 10.27 | 11.66 | 9.57 | 9.92 |
| 7 [symbol] | 13.51 | 6 | 7.48 | 8.2 | 9.9 | N/A | N/A | N/A | N/A | N/A | N/A | N/A | N/A | N/A | N/A | N/A | N/A |
| 8 [symbol] | 14.77 | 5 | [symbol] | [symbol] | [symbol] | N/A | N/A | N/A | N/A | N/A | N/A | N/A | N/A | N/A | N/A | N/A | N/A |
| 9 [symbol] | 13.67 | 35 | 7.66 | [symbol] | [symbol] | N/A | N/A | N/A | N/A | N/A | N/A | N/A | N/A | N/A | N/A | N/A | N/A |
| 10 [symbol] | [symbol] | 19.81 | [symbol] | 35 | 8.84 | N/A | N/A | N/A | N/A | N/A | N/A | N/A | N/A | N/A | N/A | N/A | N/A |
| 11 [symbol] | 20.07 | 6 | [symbol] | [symbol] | 35 | N/A | N/A | N/A | N/A | N/A | N/A | N/A | N/A | N/A | N/A | N/A | N/A |
| 12 [symbol] | 14.6 | 7.6 | [symbol] | [symbol] | [symbol] | N/A | N/A | N/A | N/A | N/A | N/A | N/A | N/A | N/A | N/A | N/A | N/A |
| 13 [symbol] | 15.62 | 8.87 | 8.76 | 6.84 | 7.45 | N/A | N/A | N/A | N/A | N/A | N/A | N/A | N/A | N/A | N/A | N/A | N/A |
| 14 [symbol] | [symbol] | 35 | 9.1 | 7.02 | 7.77 | N/A | N/A | N/A | N/A | N/A | N/A | N/A | N/A | N/A | N/A | N/A | N/A |
| 15 [symbol] | 15.33 | [symbol] | [symbol] | [symbol] | 7.91 | N/A | N/A | N/A | N/A | N/A | N/A | N/A | N/A | N/A | N/A | N/A | N/A |
| 16 [symbol] | 14.06 | 9.48 | 9.66 | 7.51 | 7.15 | N/A | N/A | N/A | N/A | N/A | N/A | N/A | N/A | N/A | N/A | N/A | N/A |
| 17 [symbol] | 15.6 | 9.53 | 10.14 | 7.8 | 8.02 | N/A | N/A | N/A | N/A | N/A | N/A | N/A | N/A | N/A | N/A | N/A | N/A |
| 18 [symbol] | 16.87 | 8.83 | 8.58 | 6.72 | 7.05 | N/A | N/A | N/A | N/A | N/A | N/A | N/A | N/A | N/A | N/A | N/A | N/A |
| 19 [symbol] | 14.46 | 9.43 | 9.77 | 7.64 | 8.03 | N/A | N/A | N/A | N/A | N/A | N/A | N/A | N/A | N/A | N/A | N/A | N/A |
| 20 [symbol] | 14.26 | 9.61 | 9.73 | 7.45 | [symbol] | N/A | N/A | N/A | N/A | N/A | N/A | N/A | N/A | N/A | N/A | N/A | N/A |
| 21 [symbol] | 13.67 | 9.51 | 9.14 | 7.33 | [symbol] | N/A | N/A | N/A | N/A | N/A | N/A | N/A | N/A | N/A | N/A | N/A | N/A |
| 22 [symbol] | [symbol] | 12.7 | 8.98 | 6.99 | 7.97 | N/A | N/A | N/A | N/A | N/A | N/A | N/A | N/A | N/A | N/A | N/A | N/A |
| 23 [symbol] | N/A | N/A | N/A | N/A | N/A | 9.91 | 12.94 | 14.02 | [symbol] | 11.62 | 11.27 | N/A | N/A | N/A | N/A | N/A | N/A |
| 24 [symbol] | N/A | N/A | N/A | N/A | N/A | 13.61 | 13.36 | 11.19 | [symbol] | 11.85 | 11.54 | N/A | N/A | N/A | N/A | N/A | N/A |
| 25 [symbol] | N/A | N/A | N/A | N/A | N/A | 12.62 | [symbol] | 12.11 | 11.53 | 11.47 | 11.78 | N/A | N/A | N/A | N/A | N/A | N/A |
| 26 [symbol] | N/A | N/A | N/A | N/A | N/A | 10.84 | 12 | 12.09 | 11.96 | 10.83 | 11.13 | N/A | N/A | N/A | N/A | N/A | N/A |
| 27 [symbol] | N/A | N/A | N/A | N/A | N/A | 12.29 | 12.02 | 12.76 | 29.5 | 11.27 | 11.29 | N/A | N/A | N/A | N/A | N/A | N/A |
| 28 [symbol] | N/A | N/A | N/A | N/A | N/A | 8.43 | 13.76 | 11.92 | 9.75 | 17.77 | 10.45 | N/A | N/A | N/A | N/A | N/A | N/A |
| 29 [symbol] | N/A | N/A | N/A | N/A | N/A | 9.6 | 11.33 | 12.27 | [symbol] | 10.3 | [symbol] | N/A | N/A | N/A | N/A | N/A | N/A |
| 30 [symbol] | N/A | N/A | N/A | N/A | N/A | 9.34 | 10.91 | 9.57 | 8.49 | 13.65 | 9.69 | N/A | N/A | N/A | N/A | N/A | N/A |
| 31 [symbol] | N/A | N/A | N/A | N/A | N/A | 8.74 | 12.11 | [symbol] | 10.23 | 18.95 | 10.99 | N/A | N/A | N/A | N/A | N/A | N/A |
| 32 [symbol] | N/A | N/A | N/A | N/A | N/A | 9.04 | 11.77 | 11.61 | 11.66 | 15.08 | 11.6 | N/A | N/A | N/A | N/A | N/A | N/A |
| 33 [symbol] | N/A | N/A | N/A | N/A | N/A | N/A | N/A | N/A | N/A | N/A | N/A | 11.65 | 9.17 | 13.11 | 16.09 | 29.26 | 11.76 |
| 34 [symbol] | N/A | N/A | N/A | N/A | N/A | N/A | N/A | N/A | N/A | N/A | N/A | 12.53 | 9.64 | 10.35 | 18.54 | 11.64 | 13.14 |
| 35 [symbol] | N/A | N/A | N/A | N/A | N/A | N/A | N/A | N/A | N/A | N/A | N/A | 11.47 | [symbol] | 10.34 | 14.63 | 29.11 | 11.73 |
| 36 [symbol] | N/A | N/A | N/A | N/A | N/A | N/A | N/A | N/A | N/A | N/A | N/A | [symbol] | 9.29 | 11.44 | 17.33 | 13.31 | 13.53 |
| 37 [symbol] | N/A | N/A | N/A | N/A | N/A | N/A | N/A | N/A | N/A | N/A | N/A | 14.01 | 9.92 | 11.69 | [symbol] | 28.95 | 12.84 |
| 38 [symbol] | N/A | N/A | N/A | N/A | N/A | 16.58 | 17.47 | 17.12 | 16.01 | 40 | 28 | N/A | N/A | N/A | N/A | N/A | N/A |
| 39 [symbol] | N/A | N/A | N/A | N/A | N/A | 28 | 40 | 40 | 28 | 19.7 | 28 | N/A | N/A | N/A | N/A | N/A | N/A |
| 40 [symbol] | N/A | N/A | N/A | N/A | N/A | 28 | [symbol] | 18.98 | 28 | [symbol] | 21.74 | N/A | N/A | N/A | N/A | N/A | N/A |
| 41 [symbol] | N/A | N/A | N/A | N/A | N/A | 40 | 40 | 40 | [symbol] | 28 | 28 | N/A | N/A | N/A | N/A | N/A | N/A |
| 42 [symbol] | N/A | N/A | N/A | N/A | N/A | [symbol] | [symbol] | 18.01 | 40 | 26 | 28 | N/A | N/A | N/A | N/A | N/A | N/A |
| 43 [symbol] | N/A | N/A | N/A | N/A | N/A | 18.03 | 17.3 | 40 | 19.57 | 16.05 | 22.87 | N/A | N/A | N/A | N/A | N/A | N/A |
| 44 [symbol] | N/A | N/A | N/A | N/A | N/A | 40 | 40 | 40 | 40 | 40 | N/A | N/A | N/A | N/A | N/A | N/A | N/A |
| 45 [symbol] | N/A | N/A | N/A | N/A | N/A | 40 | 40 | 40 | 40 | 40 | N/A | N/A | N/A | N/A | N/A | N/A | N/A |
| 46 [symbol] | N/A | N/A | N/A | N/A | N/A | 40 | 40 | 40 | 40 | 40 | N/A | N/A | N/A | N/A | N/A | N/A | N/A |
| 47 [symbol] | N/A | N/A | N/A | N/A | N/A | 40 | 40 | 40 | 40 | 40 | N/A | N/A | N/A | N/A | N/A | N/A | N/A |
| 48 [symbol] | N/A | N/A | N/A | N/A | N/A | 14.62 | 15.06 | 15.32 | 13.87 | 14.6 | N/A | N/A | N/A | N/A | N/A | N/A | N/A |

[symbol]

Nested qPCR performed with undiluted multiplex product. May skew results due to extremely early fluorescence. [symbol]
Design changes occurred only in Multiplex Group 1. Groups 2 and 3 remained unchanged during this period. [symbol]
Design changes occurred only in Multiplex Group 2. Groups 1 and 3 remained unchanged during this period. [symbol]
Design changes occurred only in Multiplex Group 3. Groups 1 and 2 remained unchanged during this period. [symbol]
indicates data missing or illegible when filed

TABLE A

| rpoB | |
|---|---|
| Codon | |
| 170 | Valine to Phenylalanine |
| 286 | Alanine to Valine |
| 359 | Valine to Alanine |
| 400 | Threonine to Alanine |
| 424 | Phenylalanine to Leucine |
| 424 | Phenylalanine to Serine |
| 424 | Phenylalanine to Valine |
| 425 | Phenylalanine Deletion |
| 426 | Glycine Deletion |
| 427 | Threonine Deletion |
| 428 | Serine Deletion |
| 429 | Glutamine Deletion |
| 430 | Leucine Deletion |
| 431 | Serine to Threonine |
| 432 | Glutamine Deletion |
| 432 | Glutamine to Histidine |
| 432 | Glutamine to Lysine |
| 432 | Glutamine to Leucine |
| 432 | Glutamine to Proline |
| 433 | Phenylalanine Deletion |
| 433 | Phenylalanine Duplication |
| 434 | Methionine Deletion |
| 434 | Methionine to Isoleucine |
| 435 | Aspartic Acid Deletion |
| 435 | Aspartic acid to Tyrosine |
| 435 | Aspartic acid to Alanine |
| 435 | Aspartic acid to Glycine |
| 435 | Aspartic acid to insertion |
| 435 | Aspartic acid to Asparagine |
| 435 | Aspartic acid to Valine |
| 436 | Glutamine Deletion |
| 437 | Asparagine Deletion |
| 438 | Asparagine Deletion |
| 439 | Proline Deletion |
| 440 | Leucine Deletion |
| 441 | Serine Deletion |
| 441 | Serine to Glutamine |
| 442 | Glycine Deletion |
| 443 | Leucine Deletion |
| 444 | Threonine Deletion |
| 445 | Histidine Deletion |
| 445 | Histidine to Cysteine |
| 445 | Histidine to Aspartic acid |
| 445 | Histidine to Phenylalanine |
| 445 | Histidine to Glycine |
| 445 | Histidine to Leucine |
| 445 | Histidine to Arginine |
| 445 | Histidine to Tyrosine |
| 446 | Lysine Deletion |
| 447 | Arginine Deletion |
| 448 | Arginine Deletion |
| 449 | Leucine Deletion |
| 450 | Serine to Leucine |
| 450 | Serine to Phenylalanine |
| 450 | Serine to Leucine |
| 450 | Serine to Glutamine |
| 450 | Serine to Tryptophan |
| 450 | Serine to Tyrosine |
| 451 | Alanine Deletion |
| 452 | Leucine Deletion |
| 452 | Leucine to Proline |
| 454 | Proline to Histidine |
| 454 | Proline to Leucine |
| 460 | Glutamic Acid to Glycine |
| 480 | Isoleucine to Threonine |
| 480 | Isoleucine to Valine |
| 491 | Isoleucine to Phenylalanine |
| 493 | Serine to Leucine |
| 513 | Glutamine to Lysine |
| 513 | Glutamine to Leucine |
| 513 | Glutamine to Proline |
| 514 | Phenylalanine duplicate |
| 516 | Aspartic Acid to Alanine |
| 516 | Aspartic Acid to Phenylalanine |
| 516 | Aspartic Acid to Glycine |
| 516 | Aspartic Acid to Valine |
| 516 | Aspartic Acid to Tyrosine |
| 518 | Asparagine deletion |
| 522 | Serine to Leucine |
| 526 | Histidine to Cysteine |
| 526 | Histidine to Proline |
| 526 | Histidine to Aspartic Acid |
| 526 | Histidine to Glycine |
| 526 | Histidine to Leucine |
| 526 | Histidine to Arginine |
| 526 | Histidine to Tyrosine |
| 531 | Serine to Phenylalanine |
| 531 | Serine to Leucine |
| 531 | Serine to Tryptophan |
| 533 | Leucine to Proline |
| rpsL. | |
| Codon | |
| 40 | Threonine to Isoleucine |
| 43 | Lysine Deletion |
| 43 | Lysine to Arginine |
| 43 | Lysine to Threonine |
| 88 | Lysine Deletion |
| 88 | Lysine to Glutamine |
| 88 | Lysine to Arginine |
| tlyA | |
| Nucleotide | |
| −83 | C to T |
| 7 | C to T |
| 26 | Frameshift |
| 52 | C to T |
| 64 | C to T |
| 200 | C to A |
| 353 | T to C |
| 383 | T to A |
| 397 | C insertion Frameshift |
| 555 | T to G |
| 758 | Frameshift |
| Codon | |
| 236 | Asparagine to Lysine |
| rv0678 | |
| Codon | |
| 63 | Serine to Arginine |
| fabG1 | |
| Nucleotide | |
| −8 | T Deletion |
| −15 | C Deletion |
| −15 | C to T |
| −16 | A Deletion |
| −17 | G to T |
| gyrA | |
| Codon | |
| 70 | Histidine to Arginine |
| 74 | Alanine to Serine |
| 85 | Histidine Deletion |
| 86 | Proline Deletion |
| 87 | Histidine Deletion |
| 88 | Glycine to Cysteine |
| 88 | Glycine Deletion |
| 89 | Aspartic Acid to Asparagine |
| 89 | Aspartic Acid Deletion |
| 90 | Alanine to Valine |
| 90 | Alanine Deletion |
| 91 | Serine to Proline |
| 91 | Serine Deletion |
| 92 | Isoleucine Deletion |
| 93 | Tyrosine Deletion |
| 94 | Aspartic Acid to Alanine |
| 94 | Aspartic Acid to Glycine |
| 94 | Aspartic Acid to Asparagine |
| 94 | Aspartic Acid to Histidine |
| 94 | Aspartic Acid Deletion |
| 96 | Leucine Deletion |
| 97 | Valine Deletion |
| eis | |
| Nucleotide | |
| −14 | C to T |
| −10 | G to A |
| embB | |
| Codon | |
| 296 | Asparagine to Histidine |
| 297 | Serine to Alanine |
| 306 | Methionine Deletion |
| 313 | Alanine to Valine |
| 319 | Tyrosine to Cysteine |
| 319 | Tyrosine to Serine |
| 328 | Aspartic Acid to Glycine |
| 328 | Aspartic Acid to Valine |
| 328 | Aspartic Acid to Tyrosine |
| 334 | Tyrosine to Histidine |
| 347 | Serine to Isoleucine |
| 354 | Aspartic Acid to Alanine |
| 356 | Alanine to Valine |
| 377 | Valine to Glycine |
| 378 | Glutamic Acid to Alanine |
| 397 | Proline to Threonine |
| 405 | Glutamic Acid to Aspartic Acid |
| 406 | Glycine to Alanine |
| 406 | Glycine to Cysteine |
| 406 | Glycine to Aspartic Acid |
| 406 | Glycine to Serine |
| 497 | Glutamine to Lysine |
| 497 | Glutamine to Proline |
| 497 | Glutamine to Arginine |
| 504 | Glutamic Acid to Aspartic Acid |
| rrs | |
| Nucleotide | |
| 905 | C to A |
| 905 | C to G |
| 906 | A to G |
| 907 | A to C |
| 907 | A to T |
| 908 | A to G |
| 1239 | T to C |
| 1325 | A to C |
| 1338 | A to C |
| 1401 | A to G |
| 1401 | A Deletion |
| 1402 | C to T |
| 1402 | C Deletion |
| 1484 | G to Deletion |
| 1484 | G to T |
| ethA | |
| Codon | |
| 1 | Methionine to Arginine |
| 21 | Isoleucine to Threonine |
| 21 | Isoleucine to Valine |
| 43 | Glycine to Cysteine |
| 61 | Threonine to Methionine |
| 232 | Threonine to Alanine |
| 338 | Isoleucine to Serine |
| 342 | Threonine to Lysine |
| 381 | Alanine to Proline |
| rplC | |
| Codon | |
| 154 | Cysteine to Arginine |
| katG | |
| Codon | |
| 155 | Tyrosine to Cysteine |
| 155 | Tyrosine to Serine |
| 159 | Leucine to Proline |
| 180 | Threonine to Lysine |
| 182 | Glycine to Arginine |
| 191 | Tryptophan to Glycine |
| 191 | Tryptophan to Arginine |
| 232 | Proline to Arginine |
| 257 | Methionine to Isoleucine |
| 275 | Threonine to Alanine |
| 295 | Glutamine to Proline |
| 297 | Glycine to Valine |
| 299 | Glycine to Cysteine |
| 300 | Tryptophan to Cysteine |
| 300 | Tryptophan to Serine |
| 302 | Serine to Arginine |
| 311 | Aspartic Acid to Glycine |
| 315 | Serine to Isoleucine |
| 315 | Serine to Asparagine |
| 315 | Serine to Threonine |
| 315 | Serine deletion |
| 321 | Tryptophan to Stop Codon |
| 328 | Tryptophan to Leucine |
| 335 | Isoleucine to Valine |
| 378 | Leucine to Proline |
| 379 | Alanine to Valine |
| 419 | Aspartic Acid to Histidine |
| 424 | Alanine to Glycine |
| gidB | |
| Codon | |
| 11 | Isoleucine to Asparagine |
| 19 | Alanine to Proline |
| 26 | Leucine to Phenylalanine |
| 30 | Glycine to Aspartic Acid |
| 34 | Glutamine to Valine |
| 41 | Valine to Isoleucine |
| 47 | Arginine to Tryptophan |
| 48 | Histidine to Asparagine |
| 48 | Histidine to Glutamine |
| 52 | Cysteine to Phenylalanine |
| 64 | Arginine to Tryptophan |
| 65 | Valine to Glycine |
| 69 | Glutamine to Aspartic Acid |
| 70 | Serine to Asparagine |
| 73 | Glycine to Alanine |
| 75 | Proline to Leucine |
| 75 | Proline to Arginine |
| 79 | Leucine to Serine |
| 79 | Leucine to Tryptophan |
| 80 | Alanine to Proline |
| 83 | Arginine to Proline |
| 85 | Aspartic Acid to Alanine |
| 88 | Valine to Alanine |
| 91 | Leucine to Proline |
| 92 | Glutamic Acid to Aspartic Acid |
| 93 | Proline to Leucine |
| 117 | Glycine to Valine |
| 118 | Arginine to Leucine |
| 118 | Arginine to Serine |
| 125 | Glutamine to Stop Codon |
| 134 | Alanine to Glutamic Acid |
| 136 | Serine to Stop Codon |
| 137 | Arginine to Proline |
| 137 | Arginine to Tryptophan |
| 138 | Alanine to Threonine |
| 138 | Alanine to Valine |
| 149 | Serine to Arginine |
| 162 | Isoleucine to Serine |
| 173 | Glutamic Acid to Stop Codon |
| 195 | Tyrosine to Histidine |
| 200 | Alanine to Glutamic Acid |
| 203 | Valine to Leucine |
| 205 | Alanine to Glutamic Acid |
| pncA | |
| Nucleotide | |
| −12 | T to C |
| −11 | A to G |
| −7 | T to C |
| Codon | |
| 1 | Methionine to Threonine |
| 3 | Alanine to Glutamic Acid |
| 4 | Leucine to Serine |
| 6 | Isoleucine to Threonine |
| 7 | Valine to Glycine |
| 8 | Aspartic Acid to Glycine |
| 8 | Aspartic Acid to Asparagine |
| 8 | Aspartic Acid to Glutamic Acid |
| 9 | Valine to Alanine |
| 10 | Glutamine to Arginine |
| 10 | Glutamine to Proline |
| 10 | Glutamine deletion |
| 12 | Aspartic Acid to Alanine |
| 12 | Aspartic Acid to Asparagine |
| 14 | Cysteine to Arginine |
| 14 | Cysteine deletion |
| 14 | Cysteine to Glycine |
| 14 | Cysteine to Tyrosine |
| 17 | Glycine to Aspartic Acid |
| 19 | Leucine to Proline |
| 21 | Valine to Glycine |
| 24 | Glycine to Aspartic Acid |
| 27 | Leucine to Proline |
| 32 | Serine to Isoleucine |
| 34 | Tyrosine deletion |
| 34 | Tyrosine to Aspartic Acid |
| 35 | Leucine to Arginine |
| 46 | Alanine to Valine |
| 46 | Alanine to Glutamic Acid |
| 47 | Threonine to Alanine |
| 47 | Threonine to Proline |
| 48 | Lysine to Threonine |
| 49 | Aspartic Acid to Alanine |
| 49 | Aspartic Acid to Glycine |
| 49 | Aspartic Acid to Asparagine |
| 51 | Histidine to Glutamine |
| 51 | Histidine to Arginine |
| 51 | Histidine to Tyrosine |
| 54 | Proline to Serine |
| 54 | Proline to Leucine |
| 57 | Histidine to Aspartic Acid |
| 57 | Histidine to Proline |
| 57 | Histidine to Arginine |
| 57 | Histidine to Tyrosine |
| 58 | Phenylalanine to Leucine |
| 58 | Phenylalanine to Serine |
| 59 | Serine to Proline |
| 61 | Threonine to Proline |
| 62 | Proline to Glutamine |
| 62 | Proline to Leucine |
| 63 | Aspartic Acid to Glycine |
| 63 | Aspartic Acid to Alanine |
| 64 | Tyrosine to Aspartic Acid |
| 66 | Serine to Proline |
| 67 | Serine to Proline |
| 68 | Tryptophan to Cysteine |
| 68 | Tryptophan to Arginine |
| 68 | Tryptophan to Glycine |
| 69 | Proline to Leucine |
| 71 | Histidine to Tyrosine |
| 71 | Histidine to Glutamine |
| 71 | Histidine to Arginine |
| 71 | Histidine to Aspartic Acid |
| 72 | Cysteine to Arginine |
| 72 | Cysteine to Tyrosine |
| 76 | Threonine to Proline |
| 76 | Threonine to Isoleucine |
| 78 | Glycine to Cysteine |
| 78 | Glycine to Aspartic Acid |
| 81 | Phenylalanine to Valine |
| 82 | Histidine to Arginine |
| 82 | Histidine to Aspartic Acid |
| 85 | Leucine to Proline |
| 85 | Leucine to Arginine |
| 87 | Threonine to Methionine |
| 90 | Isoleucine to Serine |
| 94 | Phenylalanine to Leucine |
| 94 | Phenylalanine to Serine |
| 96 | Lysine to Asparagine |
| 96 | Lysine to Arginine |
| 96 | Lysine to Glutamic Acid |
| 96 | Lysine to Threonine |
| 97 | Glycine to Aspartic Acid |
| 97 | Glycine to Cysteine |
| 97 | Glycine to Serine |
| 99 | Tyrosine deletion |
| 102 | Alanine to Valine |
| 103 | Tyrosine duplication |
| 103 | Tyrosine deletion |
| 103 | Tyrosine to Histidine |
| 104 | Serine to Arginine |
| 104 | Serine to Glycine |
| 108 | Glycine to Arginine |
| 114 | Threonine to Proline |
| 116 | Leucine to Proline |
| 116 | Leucine to Arginine |
| 120 | Leucine to Proline |
| 123 | Arginine to Proline |
| 125 | Valine to Phenylalanine |
| 125 | Valine to Glycine |
| 128 | Valine to Glycine |
| 130 | Valine to Glycine |
| 132 | Glycine to Alanine |
| 132 | Glycine to Aspartic Acid |
| 132 | Glycine to Serine |
| 133 | Isoleucine to Threonine |
| 134 | Alanine to Valine |
| 135 | Threonine to Proline |
| 135 | Threonine to Asparagine |
| 137 | Histidine to Proline |
| 137 | Histidine to Arginine |
| 138 | Cysteine to Arginine |
| 138 | Cysteine to Serine |
| 138 | Cysteine to Tyrosine |
| 139 | Valine to Glycine |
| 139 | Valine to Leucine |
| 139 | Valine to Alanine |
| 139 | Valine to Methionine |
| 141 | Glutamine to Proline |
| 141 | Glutamine deletion |
| 142 | Threonine to Alanine |
| 142 | Threonine to Lysine |
| 142 | Threonine to Methionine |
| 146 | Alanine to Threonine |
| 146 | Alanine to Valine |
| 148 | Indel Arginine insert (in frame) |
| 151 | Leucine to Serine |
| 154 | Arginine to Glycine |
| 155 | Valine to Glycine |
| 155 | Valine to Alanine |
| 155 | Valine to Leucine |
| 159 | Leucine to Valine |
| 159 | Leucine to Proline |
| 160 | Threonine to Proline |
| 161 | Alanine to Proline |
| 162 | Glycine to Aspartic Acid |
| 168 | Threonine to Proline |
| 171 | Alanine to Glutamic Acid |
| 172 | Leucine to Proline |
| 175 | Methionine to Threonine |
| 175 | Methionine to Valine |
| 180 | Valine to Phenylalanine |
| 180 | Valine to Glycine |
| rrl | |
| Nucleotide | |
| 2058 | G Deletion |
| inhA | |
| Nucleotide | |
| −15 | C to T |
| Codon | |
| 21 | Isoleucine to Threonine |
| 21 | Isoleucine to Valine |
| 49 | Serine to Alanine |
| 194 | Isoleucine to Threonine |

1. An oligonucleotide for amplifying a portion of the gene inhA from M. tuberculosis and/or related bacteria in the M. tuberculosis complex, comprising or consisting of a forward primer specific for said portion, wherein the forward primer has a sequence as set out in: SEQ ID No. 23, SEQ ID No. 35, SEQ ID No. 36, SEQ ID No. 37 or SEQ ID No. 38.

2. An oligonucleotide primer set for amplifying a portion of the gene inhA from M. tuberculosis and/or related bacteria in the M. tuberculosis complex, wherein the set comprises or consists of a pair of forward and reverse primers specific for said portion, wherein the set comprises or consists of a forward primer as claimed in claim 1, and a reverse primer having a sequence as set out in SEQ ID No. 24.

3. One or more oligonucleotide primer sets for amplifying a portion of one or more genes from M. tuberculosis and/or related bacteria in the M. tuberculosis complex selected from the group comprising one or more of eis, embB, ethA, fabG1, gidB, gyrA, inhA, katG, pncA, rrl, rplC, rpoB, rpsL, rrs, rv0678 and tlyA, wherein each set comprises or consists of a pair of forward and reverse primers specific for said portion, wherein each primer has a sequence as set out in SEQ ID Nos. 1-32 or 35-38.

4. Oligonucleotide primer sets as claimed in claim 3, for use in multiplex PCR, wherein the primer sets are grouped into one or more multiplex groups, wherein the groups comprise at least two primer sets selected from: SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24, optionally wherein the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24.

5. A group of oligonucleotide primer sets for use in multiplex PCR as claimed in claim 4 comprising each of the oligonucleotide primer sets set out in SEQ ID Nos. 1 to 32; or each of the oligonucleotide primer sets set out in SEQ ID Nos. 1 to 22, 24 to 32 and 35; or each the oligonucleotide primer sets set out in of SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38.

6. A group of oligonucleotide primer sets for use in multiplex PCR as claimed in claim 4, comprising each of SEQ ID Nos. 1 to 32.

7. An oligonucleotide primer set or a group of oligonucleotide primer sets as claimed in claim 3, wherein the portion of the one or more genes contains one or more mutations that confer antibiotic resistance to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinoloes, preferably wherein the one or mutations are one or more single nucleotide polymorphisms.

8. A multiplex PCR reaction mixture comprising a group of oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inhA, katG, pncA, rrl, rplC, rpoB, rpsL, rrs, rv0678, tlyA, wherein each set comprises a pair of forward and reverse primers specific for said portion, wherein the group of oligonucleotide primer sets comprises at least two primer sets selected from SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24, optionally wherein the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24.

9. A multiplex PCR reaction mixture as claimed in claim 8 comprising each of the oligonucleotide primer sets set out in SEQ ID Nos. 1 to 32; or each of the oligonucleotide primer sets set out in SEQ ID Nos. 1 to 22, 24 to 32 and 35; or each of the oligonucleotide primer sets set out in SEQ ID Nos. 1 to 22, 24 to 32 and 36; or each of the oligonucleotide primer sets set out in SEQ ID Nos. 1 to 22, 24 to 32 and 37; or each of the oligonucleotide primer sets set out in SEQ ID Nos. 1 to 22, 24 to 32 and 38.

10. A multiplex PCR reaction mixture as claimed in claim 8, comprising each of SEQ ID Nos. 1 to 32.

11. A method of detecting the presence of one or more mutations that confer antibiotic resistance in a sample comprising DNA from Mycobacterium tuberculosis and/or related bacteria in the M. tuberculosis complex, said method including the steps of:
(a) isolating or extracting DNA from the sample;
(b) amplifying relevant gene regions or amplicons by polymerase chain reaction using one or more oligonucleotide primer sets as claimed in claim 2;
(c) subjecting the amplified gene regions or amplicons to DNA sequencing; and
(d) detecting one or more mutations.

12. A method of predicting whether a patient suffering from tuberculosis will respond to treatment with one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones, said method comprising a step of determining the presence of one or more drug resistant mutations in one or more genes selected from the group comprising one or more of eis, embB, ethA, fabG1, gidB, gyrA, inhA, katG, pncA, rrl, rplC, rpoB, rpsL, rrs, rv0678 and tlyA in DNA obtained from a sample from the patient, the method comprising:
(a) isolating or extracting DNA from the sample;
(b) amplifying relevant gene regions or amplicons by polymerase chain reaction using one or more oligonucleotide primer sets as claimed in claim 2;
(c) subjecting the amplified gene regions or amplicons to DNA sequencing; and
(d) detecting the one or more mutations.

13. A method as claimed in claim 11, wherein detection of:
(i) a mutation in embB using an oligonucleotide primer set comprising SEQ ID Nos. 3 and 4 indicates resistance to ethambutol;
(ii) a mutation in fabG1 using an oligonucleotide primer set comprising SEQ ID Nos. 9 and 10; a mutation in inhA using an oligonucleotide primer set comprising SEQ ID Nos. 23 and 24; SEQ ID Nos. 35 and 24; SEQ ID Nos. 36 and 24; SEQ ID Nos. 37 and 24 or SEQ ID Nos. 38 and 24; and/or a mutation in katG using an oligonucleotide primer set comprising SEQ ID Nos. 19 and 20 indicates resistance to isoniazid;
(iii) a mutation in pncA using an oligonucleotide primer set comprising SEQ ID Nos. 27 and 28 indicates resistance to pyrazinamide;
(iv) a mutation in rpoB using an oligonucleotide primer set comprising SEQ ID Nos. 13 and 14 indicates resistance to rifampicin;
v) a mutation in gidB using an oligonucleotide primer set comprising SEQ ID Nos. 21 and 22; a mutation in rpsL using an oligonucleotide primer set comprising SEQ ID Nos. 29 and 30; and/or a mutation in rrs using an oligonucleotide primer set comprising SEQ ID Nos. 5 and 6 indicates resistance to streptomycin;
(vi) a mutation in rrs using an oligonucleotide primer set comprising SEQ ID Nos. 5 and 6 indicates resistance to amikacin;
(vii) a mutation in rv0678 using an oligonucleotide primer set comprising SEQ ID Nos. 7 and 8 indicates resistance to bedaquiline and/or clofazimine;
(viii) a mutation in gidB using an oligonucleotide primer set comprising SEQ ID Nos. 21 and 22; a mutation in rrs using an oligonucleotide primer set comprising SEQ ID Nos. 5 and 6; and/or a mutation in tlyA using an oligonucleotide primer set comprising SEQ ID Nos. 31 and 32 indicates resistance to capreomycin;
(ix) a mutation in gyrA using an oligonucleotide primer set comprising SEQ ID Nos. 11 and 12 indicates resistance to ciprofloxacin;
(x) a mutation in ethA using an oligonucleotide primer set comprising SEQ ID Nos 15 and 16; a mutation in fabG1 using an oligonucleotide primer set comprising SEQ ID Nos. 9 and 10, and/or a mutation in inhA using an oligonucleotide primer set comprising SEQ ID Nos. 23 and 24; SEQ ID Nos. 35 and 24; SEQ ID Nos. 36 and 24; SEQ ID Nos. 37 and 24 or SEQ ID Nos. 38 and 24 indicates resistance to ethionamide;
(xi) a mutation in eis using an oligonucleotide primer set comprising SEQ ID Nos. 1 and 2 and/or a mutation in rrs using an oligonucleotide primer set comprising SEQ ID Nos. 5 and 6 indicates resistance to kanamycin;
(xii) a mutation in rplC using an oligonucleotide primer set comprising SEQ ID Nos. 17 and 18 indicates resistance to linezoild;
(xiii) a mutation in gyrA using an oligonucleotide primer set comprising SEQ ID Nos. 11 and 12 indicates resistance to moxifloxacin, ofloxacin and/or quinolones.

14. A method as claimed in claim 11, wherein step (b) involves amplifying relevant gene regions or amplicons by multiplex PCR reaction using a multiplex PCR reaction mixture comprising a group of oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inhA, katG, pncA, rrl, rplC, rpoB, rpsL, rrs, rv0678, tlyA, wherein each set comprises a pair of forward and reverse primers specific for said portion, wherein the group of oligonucleotide primer sets comprises at least two primer sets selected from SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24, optionally wherein the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24.

15. A method as claimed in claim 11, wherein the sample is one or more tissues and/or bodily fluids obtained from a subject suspected of having, or confirmed to have TB, optionally wherein the sample is sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB.

16. A method for determining an appropriate antibiotic treatment regime for a patient with tuberculosis, comprising detecting and/or identifying the presence of one or more mutations that confer antibiotic resistance in a sample from the subject using the method as claimed in claim 11, and determining an appropriate antibiotic regime on the basis of the mutations detected/identified.

17. A kit comprising one or more oligonucleotide primer sets or oligonucleotide primer set groups as claimed in claim 2, or a multiplex PCR reaction mixture comprising a group of oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inhA, katG, pncA, rrl, rplC, rpoB, rpsL, rrs, rv0678, tlyA, wherein each set comprises a pair of forward and reverse primers specific for said portion, wherein the group of oligonucleotide primer sets comprises at least two primer sets selected from SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 35 and 24; 36 and 24; 37 and 24; and 38 and 24, optionally wherein the group of oligonucleotide primer sets comprises at least SEQ ID Nos. 23 and 24; SEQ ID Nos. 35 and 24; SEQ ID Nos. 36 and 24; SEQ ID Nos. 37 and 24; or SEQ ID Nos. 38 and 24.
